(19) United States
(12) Patent Application Publication
(10) Pub. No.: US 2024/0320935 A1
(43) Pub. Date: Sep. 26, 2024

(54) Systems, Methods and Devices for Augmented Reality Assisted Surgery

(71) Applicant: Materialise N.V., Leuven (BE)

(72) Inventors: Andrei PISSARENKO, Leuven (BE); Christophe VAN DIJCK, Leuven (BE); Durva GAJJAR, Leuven (BE); Pieter SLAGMOLEN, Leuven (BE); Veerle PATTIJN, Leuven (BE)

(21) Appl. No.: 18/657,417

(22) Filed: May 7, 2024

Related U.S. Application Data
(63) Continuation of application No. PCT/US22/49732, filed on Nov. 11, 2022.
(60) Provisional application No. 63/264,022, filed on Nov. 12, 2021.

(51) Int. Cl. G06T 19/00; A61B 34/10; A61B 34/20

(52) U.S. Cl. CPC G06T 19/006; A61B 34/10; A61B 34/20; A61B 2034/102; A61B 2034/107

(57) ABSTRACT

Methods and systems are presented herein for augmented reality assisted surgery. Methods and systems include methods and systems for using physical registration objects with a known geometry for registration to determine a reference coordinate system for an augmented reality scene for a surgical setting. Methods and systems include methods and systems for providing an integrated virtual workbench for presenting and sharing patient data in a surgical setting. Methods and systems include methods and systems for providing augmented reality guidance for plate bending for craniomaxillofacial implant surgery.

### CROSS-REFERENCE TO RELATED APPLICATIONS

This application is a continuation of U.S. International Application No. PCT/US2022/49732 filed Nov. 11, 2022, which claims the benefit of and priority to U.S. Provisional Patent Application No. 63/264,022 filed on Nov. 12, 2021, the entire contents of each of which are incorporated herein by reference.

### BACKGROUND

### Technical Field

This application relates to a field of computer-assisted, image-based surgery, such as, for craniomaxillofacial surgery. Aspects of the present application relate to systems, devices, and methods for using augmented reality during surgery.

### Description of Related Technology

Several clinical indications in or around the head may lead to craniomaxillofacial surgery (CMF). The aims of such surgery can for example include the removal of malignant cells, restoring function or aesthetics and/or eliminating pain in the craniomaxillofacial region. Several surgical procedures are used, depending on the clinical indication. For example, orthognathic surgery will correct for functional or aesthetic limitations caused by malalignment of the jaw. Reconstructive surgery may, for example, be used to remove a tumor and reconstruct the anatomy to a normal state. Trauma surgery may be used to treat pain, functional loss or aesthetic problems after fractures.

In some cases, a virtual surgical plan is created that supports the surgeon in defining the desired surgical outcome. Computer assistance may be used during surgical intervention to execute that surgical plan.

Computer-assisted solutions have been studied and now implemented in practice to assist surgeons achieve accurate and safe interventions. In particular, conventional computer-assisted methods and systems exist that help a surgeon navigate routine and complex craniomaxillofacial surgeries. These systems help surgeons achieve accurate and safe interventions by providing patient-specific pre- and intra-operative guidance. These systems and methods enable the surgeon to achieve surgical precision, e.g., by avoiding damage to critical anatomical structures such as nerves, blood vessels, etc., and can sometimes shorten surgery time.

A drawback of many conventional computer-assisted surgical navigation systems that negatively influences their user friendliness is that many such systems come with bulky or cumbersome hardware, which occupies valuable space in the operating room. Also, because they are placed outside of the sterile field and working space of the surgical staff, line-of-sight issues that disturb its usage often occur. The use of traditional display systems as part of the computer assistance also requires the surgeon to focus his attention outside of the surgical field, leading to discomfort and dissociation between for example the virtual surgical plan and the patient.

Recently, new computer assisted systems including mixed reality (MR) systems or augmented reality (AR) have made their way into the operating room (OR), particularly in orthopaedic, spinal and neurosurgery, amongst others. They provide intra-operative visualization or navigation based on medical data from the patient in an augmented environment superimposed on the real world. In navigation scenarios, they help overcome the shortcomings of conventional navigation systems such as a 2-D display systems with corresponding hand-eye coordination challenges, bulky setup, line-of-sight issues or others. However, due to high demands on accuracy in craniomaxillofacial surgeries, driven by, for example, the presence of critical anatomical structures (brain, nerves, etc.) and the strong aesthetic and functional impact of the surgery, little to no adaptation and transfer of AR-based applications has occurred for craniomaxillofacial surgeries. Also, the amount of highly specialized indications is high in craniomaxillofacial surgery, and require a system which is flexible, compared to, for example, an AR navigation solution for single, clearly defined, high-volume orthopaedic procedures.

### SUMMARY

Certain embodiments provide a method of providing augmented-reality-assisted surgery. The method includes generating a virtual scene comprising one or more virtual elements comprising one or more anatomical elements corresponding to one or more anatomy parts. The method further includes registering the virtual scene to one or more references to generate an augmented scene. The method further includes displaying the augmented scene on a display device.

Certain embodiments further provide a method of providing augmented-reality-assisted surgery. The method includes generating a virtual scene comprising one or more virtual elements comprising one or more anatomical elements corresponding to one or more anatomy parts. The method further includes identifying one or more references in an actual physical scene comprising the one or more anatomy parts. The method further includes registering the virtual scene to the one or more references to generate an augmented scene. The method further includes displaying the augmented scene on a display device.

Certain embodiments provide a method of providing augmented reality assisted surgery.

Certain embodiments provide devices for use during virtual augmented realty assisted surgery.

Certain embodiments provide a system of providing a virtual workbench for use in an augmented reality assisted surgery.

Certain embodiments provide a system of providing a simple, minimalistic user interface for a virtual workbench using augmented reality.

Certain embodiments provide a method of providing a virtual workbench for use in an augmented-reality-assisted surgery. The method includes generating a virtual scene comprising one or more virtual elements comprising one or more anatomical elements corresponding to one or more anatomical parts and/or one or more instrument elements corresponding to one or more medical devices. The method further includes identifying one or more references in an actual physical scene comprising the one or more anatomy parts and/or instruments and/or medical devices. The method further includes registering the virtual scene to the one or more references to generate an augmented scene. The method further includes integration with other devices and external systems such as a fabricator or additive manufacturing unit such as a 3-D printer to facilitate manufacturing, such as 3-D printing, for example, 3-D printing one or more medical devices, scaffolds, anatomical models and/or elements in accordance with user preference, robotic arm, etc. The method further includes guiding elements corresponding to conversion of standard instruments, medical devices (such as implants) or grafts to custom (or personalized) versions, guiding of assembling, repositioning and/or fixating bone fragments (or pieces). The method further includes displaying the augmented scene on a display device.

Certain embodiments provide a system for providing a virtual workbench in an (sterile) environment for planning one or more surgical steps using augmented reality.

Certain embodiments provide a system for providing a virtual workbench in a (sterile) environment for designing one or more surgical steps using virtual elements.

Certain embodiments provide a system for providing a virtual workbench in a (sterile) environment for guiding or navigating a surgical step during a surgery using virtual elements.

Certain embodiments provide a system for providing a virtual workbench in a (sterile) environment for planning and guiding adaption of standard medical devices into customized devices using virtual elements.

Certain embodiments provide a system for providing a virtual workbench in a (sterile) environment for controlling other external systems connected to the network.

Certain embodiments provide a system for providing a virtual workbench that is configurable to integrate the virtual elements into the physical world during the process of virtual surgical planning.

Certain embodiments provide a system for providing a virtual workbench that is configurable to integrate virtual elements into the physical world during the process of preparing the OR for a surgery.

Certain embodiments provide for systems and methods of using a surgical device of known shape for registration of real world objects to virtual objects for use during an augmented reality assisted surgery.

Certain embodiments provide a method of providing virtual guidance and/or assistance during a craniomaxillofacial surgery using augmented reality.

Certain embodiments provide for systems for and methods of providing virtual guidance and/or assistance during orthognathic surgery using augmented reality.

Certain embodiments provide for systems for and methods of providing virtual guidance and/or assistance during reconstruction surgery using augmented reality.

Certain embodiments provide for systems for and methods of providing virtual guidance and/or assistance for repositioning or reconstructing bone fragments during a craniomaxillofacial surgery using augmented reality.

Certain embodiments provide for systems for providing virtual guidance and/or assistance for controlling external systems using augmented reality.

Certain embodiments provide for systems for and methods of providing virtual guidance and/or assistance for controlling an additive-manufacturing system using augmented reality.

Certain embodiments provide for systems for and methods of providing virtual guidance and/or assistance during a craniomaxillofacial surgery using an optical head-mounted display.

Certain embodiments provide a system for virtual guidance for adapting of standard medical devices into customized medical devices.

Certain embodiments provide a method of virtual guidance for adapting of standard medical devices into customised medical devices.

Certain embodiments provide virtual surgical guides configured to guide a bone cut in a craniomaxillofacial surgery.

Certain embodiments provide virtual surgical guides configured to include a virtual cut slot.

Certain embodiments provide a non-transitory computer-readable medium having computer-executable instructions stored thereon, which, when executed by a processor of a computing device, cause the computing device to perform one or more of the described methods.

Certain embodiments provide a computing device comprising a memory and a processor configured to perform one or more of the described methods.

### BRIEF DESCRIPTION OF THE DRAWINGS

FIG. 1 is a block diagram of one example of an augmented-reality (AR) system as a computing environment suitable for implementing an augmented-reality system in accordance with one or more embodiments disclosed herein.

FIG. 2 is a high-level workflow diagram for an augmented-reality system in accordance with one or more embodiments.

FIG. 3 is a high-level system diagram of a computing system that may be used in accordance with one or more embodiments.

FIGS. 4A-4B illustrate a flow chart showing a process of conventional virtual surgical planning of a craniomaxillofacial surgery.

FIG. 5 illustrates a flow chart showing a process of conventional virtual surgical planning of an orthognathic surgery.

FIG. 6 illustrates a flow chart showing a process of conventional virtual surgical planning of a reconstruction surgery of a mandible.

FIG. 7 illustrates a flow chart showing a process of adapting a medical device for operating in an augmented-reality system, according to certain embodiments.

FIG. 8 illustrates a flow chart showing a process of adapting a standard medical device into a custom (personalized) device in an augmented-reality system for a craniomaxillofacial surgery, according to certain embodiments.

FIG. 9 illustrates a flow chart showing a process of bending of a standard plate operating in an augmented-reality system for an orthognathic surgery, according to certain embodiments.

FIG. 10 illustrates a flow chart showing a process of bending of a standard plate operating in an augmented-reality system for a reconstruction surgery, according to certain embodiments.

FIGS. 11A-11C illustrate a high-level system diagram of an AR system, according to certain embodiments.

FIGS. 12A-12D illustrate an environment of a virtual workbench of an augmented-reality system, according to certain embodiments.

FIGS. 13A-13C illustrate a view of the graphical user interface of the virtual-workbench platform, according to certain embodiments.

FIGS. 14A-14D illustrate views of the different tabs of a graphical user interface of the virtual-workbench platform, according to certain embodiments.

FIGS. 15A-15F illustrate a process of reconstructing a bone graft using the virtual workbench for a reconstruction surgery, according to certain embodiments.

FIG. 16 illustrates an embodiment of the AR system.

FIG. 17 illustrates a flow chart showing a process of using the virtual workbench, according to certain embodiments.

FIG. 18 illustrates a flow chart showing a process of using the virtual workbench for a reconstructing surgery, according to certain embodiments.

FIG. 19 illustrates a flow chart showing a process of using the virtual workbench for a craniosynostosis surgery, according to certain embodiments.

FIGS. 20A-20H illustrate anatomical/cephalometric landmarks that may be used during an augmented-reality-assisted surgery, according to certain embodiments.

FIG. 21 illustrates a flow chart showing a process for operating an augmented-reality system, according to certain embodiments.

FIG. 22 illustrates a flow chart showing a process for operating an augmented-reality system, according to certain embodiments.

FIG. 23 illustrates anatomical/cephalometric landmarks used during augmented-reality-assisted orbital-floor-reconstruction surgery, according to certain embodiments

FIGS. 24A-24B illustrate anatomical/cephalometric landmarks used during augmented-reality-assisted craniosynostosis surgery, according to certain embodiments.

### DETAILED DESCRIPTION

Aspects of the disclosure describe an AR system that is configured to provide computer assistance during surgery, such as highly specialized craniomaxillofacial (CMF) surgery.

A major limitation of existing AR systems and methods related to accuracy is that they reconstruct the 3-D space on relative dimensions and lack absolute, true-to-scale dimensions of virtual objects, meaning that the surgeon still has to take into account real-world scaling and work with approximations.

Aspects of the disclosure describe an AR system which is able to provide true-to-scale dimensions of virtual objects for assisting a surgeon with specific tasks during surgery, such as CMF surgery, such as plate bending.

Another difficulty in AR is related to the difficulty identified by the inventors herein of physical registration of anatomy of a patient in order to determine a reference coordinate system for an augmented-reality scene. For example, a conceptual AR system could use the anatomy of a patient as the physical registration object to which the AR system registers to determine a reference coordinate system for AR. Virtual objects, such as virtual surgical guides, could then be displayed relative to the anatomy of the patient, based on aligning the surgical guides in the reference coordinate system based on the anatomy of the patient. A user may then try to place a physical object, such as a physical surgical guide, an implant or a surgical instrument in alignment with the virtual object displayed on the physical anatomy, to place the physical object for surgery. However, patient anatomy varies from person to person, and has many soft boundaries that may change in shape such as due to movement in tissue, fluids, etc. Moreover, not all landmarks useful for registration may be visible within the surgical window. Therefore, using the anatomy of a patient for physical registration may be difficult or not feasible.

Therefore, certain aspects herein provide techniques for instead using a physical surgical device with a known shape, such as a surgical implant, surgical instrument, or surgical guide as the physical registration object in order to determine a reference coordinate system for an augmented reality scene. The anatomy of the patient may then be used as virtual objects (e.g., virtual anatomy) displayed in a desired relative position to the physical surgical device. Accordingly, as the physical surgical device is moved by a user in the augmented reality scene, the virtual anatomy of the patient also moves, while maintaining its position relative to the physical surgical device. A user may then move the physical surgical device until the displayed virtual anatomy aligns with the physical anatomy of the patient in order to place the physical surgical device for surgery. Such aspects beneficially use a device with a known shape for physical registration, while still allowing proper alignment of surgical devices to perform surgery.

Another major limitation of existing AR systems is that they are not designed for convenient access during highly specialized surgery, leading to cluttering of the virtual space and potential interference with the surgical flow. They lack the capability of seamlessly integrating large amounts of data and digital functions into a clinical workflow. Further, certain tasks/data are relevant for a brief period during a surgery (e.g., looking at a pre-op virtual 3-D model is useful before making a cut, plate bending guidance is useful only at the time of plate bending, etc.). Current AR systems don't adapt their interface to a specific task that may be executed as part of a full surgery. This means that the user has to spend time in adjusting (or decluttering) his virtual (or augmented) environment to remove any information that he may not require.

An AR system may use multiple different and non-integrated free-floating virtual screens or objects that may be fixed spatially to a specific location in a room, e.g., using a spatial mapping technique. This may complicate the interaction of a user with the system as the user experience is determined by the relative location of the user and the virtual screens in the AR system. Especially in a dynamic environment such as a surgery room and in scenarios with multiple virtual screens, this may require the user to spend time in reorganizing the spatial locations of the virtual screens in the AR system, losing valuable time during surgery.

Certain aspects of this disclosure provide an AR system that contains a virtual workbench (VWB). A virtual workbench is a virtual, three-dimensional entity that serves as a point-of-contact between the virtual AR system and the physical world. The virtual workbench organizes data in the AR system in a relevant way in space (e.g., intelligently selecting the location of virtual elements in the OR) and time (e.g., modifying the interface of the AR system according to a task a user is performing with the AR system). The virtual workbench facilitates the interaction of a user with the virtual environment in the AR system. Similar to a physical workbench that comes with a toolbox, such as an artisan's workbench with tools, a virtual workbench provides a user access to virtual tools which are part of the AR system and which may be called upon to execute one or more tasks during a surgery, such as a CMF surgery.

Further, a typical operating room (OR) is filled with technology (or machinery). Many such systems are equipped with their own interface (e.g., diagnostic and measurement tools, displays, robots, lights, cameras, 3-D printers, AR setups, etc.). The addition of an AR system risks introducing yet another interface for the user to manage. The overload of systems which as a result takes up space in the OR complicates the easy of use (especially also as the interfaces are not uniform).

Certain aspects of this disclosure describe an AR system with a virtual workbench which circumvents this problem by flexibly and seamlessly integrating all kinds of technologies (and their interfaces) into an AR system, thereby enabling a user to configure the virtual interfaces and data according to his ad-hoc needs.

Another limitation of existing AR systems is that they may provide surgeons with the ability to perform navigation, without tackling the challenges that traditional navigation systems (without AR) experience. Often, the procedure for registration and tracking is cumbersome, or requires additional marker systems to be introduced in the patient. For example, surgery may involve the use of implants and/or implant components and the correct positioning of these implant components in relation to the bony anatomy (e.g., mandible, maxilla, orbital floor, cranium) may be crucial in achieving good patient outcome. Existing AR system may still require instruments or bone pins to be attached to the patient to perform such navigation.

Aspects of this disclosure provide an AR system which is capable of tracking objects used during surgery, such as CMF surgery (such as guides or implants) to provide a visual navigation system without requiring an explicit registration step between virtual and physical anatomy itself, or the use of marker systems on anatomy, and instead relying on registration of a physical registration object of known geometry, as further discussed herein. It can leverage the capabilities of AR to provide overlay on a specific location in the real world.

The limitations of current AR systems show that there is clearly a need for an AR system that is designed for complex surgeries such as CMF surgery, that provides seamless integration of the virtual environment with the physical environment, that improves user friendliness of the system and that is capable of providing a virtual environment for guiding, assisting, designing, planning and/or operating other integrated systems in the AR environment.

Accordingly, certain aspects of the present disclosure provide novel systems and methods for using mixed reality (e.g., augmented (AR)), to allow the translation of the virtual surgical plan to the operating room (OR) by blending the surgical scene with a virtual environment (‘augmented environment’), and using a display device/unit, such as a portable device or a headset, to visualize this blended environment, thereby assisting the surgeon. The systems and methods described herein provide improved ability for the surgeon (and optionally patient) to plan, visualize, and evaluate surgical procedures with improved accuracy as compared to other existing augmented-reality systems resulting in improved patient outcomes. The apparatuses provided herein are enhanced using augmented-reality systems and methods and are aimed at improving patient outcome.

Certain aspects of the disclosure also provide augmented-reality-assisted systems, for performing a surgical process or parts of the surgical process, and methods and apparatuses for designing and/or adapting medical devices, and in particular, shaping of implants (e.g., plates).

Certain aspects of the disclosure also provide systems for, methods of, and devices for providing a virtual workbench for use in a (e.g., sterile) surgical environment such as during an augmented-reality-assisted surgery for assisting a surgical process or parts of the surgical process.

Certain aspects of the disclosure also provide systems for, methods of and devices for virtually working at a dedicated, localized, location(s) in an (e.g., sterile) environment. The systems, methods and devices relate to a user interface that seamlessly integrates the virtual world into an operating room(s).

Certain aspects of the disclosure also provide systems that generate a three-dimensional, virtual workbench where a user performs a plurality of types of actions such as planning one or more surgical processes or parts thereof, designing one or more surgical devices or parts thereof, controlling or operating other systems or performing one or more surgical steps simultaneously or at known intervals in accordance with the surgical procedure.

A virtual workbench is a virtual representation of a physical workbench (also sometimes known as a utility toolbox) that occupies three-dimensional volume in (virtual) space. The virtual workbench facilitates the interaction of a user with the virtual environment and seamlessly integrates the virtual, augmented world into the physical, actual world. It is overlaid on the actual physical environment. It may be represented in a geometric form that occupies 3-D space, such as a rectangular, cube, cuboid, or a square virtual workbench. All the modules and components that comprise an AR system are accessible virtually using the virtual workbench as described herein.

Certain aspects of the present disclosure also provide systems for, methods of and apparatuses for repositioning or reconstructing anatomical structures or parts thereof, designing and/or adapting medical devices, and in particular, shaping of implants (e.g., plates) or grafts.

Unless otherwise mentioned, the term “operator” herein refers to the person executing the methods or method steps described herein, or operating the systems described herein. Unless otherwise mentioned, the operator may be a medical professional, a non-medical professional, such as a technician, engineer, or a trained employee. The term “operator” may also be interchangeably used with “user”.

The term “adaptable medical devices” or “adaptable medical device parts” herein refers to one or more items of medical devices or one or more parts of a medical device, respectively, that have been specifically adapted for a particular patient such as standard implants, standard plates, etc. In certain aspects, the terms “implant” or “adapted medical device” may refer to “plate(s)”. In certain aspects, the term “virtual 3D models” may refer to “virtual anatomical 3-D models” or “anatomical 3-D patient-specific models”. “Virtual 3-D models of one or more medical devices,” such as plates, guides, screws, etc., may also be referred to as “virtual medical device 3-D model(s).” The term “virtual workbench” or “virtual work bench” may also be interchangeably used with “virtual bench” or “workbench” or “work bench” or “virtual toolbox” or “utility toolbox” or “toolbox”. The terms “coordinate system” and “coordinate frame” may be used interchangeably.

The terminology native or constitutional is used to represent pre-diseased (or healthy) anatomy which may be reconstructed based on historical data and/or computer simulations of healthy individuals, e.g., driven by population knowledge or an understanding of disease progression. The terms pre-surgical, pre-operative or anatomical are used to represent the (diseased) anatomy of the patient before surgery. The term pre-operatively planned is used to represent the desired situation of the patient as it was determined using virtual planning based on medical imaging before surgery. The term intra-operatively planned is used to represent the desired situation in a new or updated plan that was created at any point during surgery based on pre-operative or intra-operative information. The term planned may refer to either pre-operatively planned or intra-operatively planned. The terms real-time or live refer to the intra-operative situation where the position of anatomy, instruments or components are tracked and used as input for a simulation process that predicts the post-operative situation or as input for the execution of the surgery or the surgical plan.

Some surgical interventions are intended to correct bone deformations, occurrences of disharmony or proportional defects of the body, in particular, the face, or post-traumatic after-effects. These interventions may use actions for repositioning, such as in a pre-operatively or intra-operatively planned location, some fragments of bone which have been separated from a base portion beforehand by trauma or by a medical professional.

Surgical interventions may therefore comprise an osteotomy which is carried out in order to release one or more badly positioned bone segments; for example, to move this or these bone segment(s), that is to say, to move it/them by way of translation and/or by rotation in order to be able to reposition it/them such as at their ideal location. In some interventions, such as trauma or reconstruction surgeries but also some corrective osteotomies, the surgical intervention may also involve the harvesting and use of one or more bone grafts. Further, osteotomies may be performed to remove- or resect-segments of the native bone that are not repositioned but removed in any case; e.g., a bone segment that has a tumor growth or a segment of a bone or bone fragment that would otherwise collide with another bone or bone fragment upon repositioning and thus prevent proper repositioning.

When all bone segments occupy a new position, the surgeon fixes the bone segments to other adjacent bone portions of the patient using one or more implants. These may comprise perforated implants, which may have different geometries, for example, in the form of I-shaped, L-shaped, T-shaped, X-shaped, H-shaped or Z-shaped plates, or more complex geometries. The implants are fixed to all the portions of bone to be joined in their correct relative positions using osteosynthesis screws which extend through their perforations. More than one combination of the above-mentioned implants may be used at the same time.

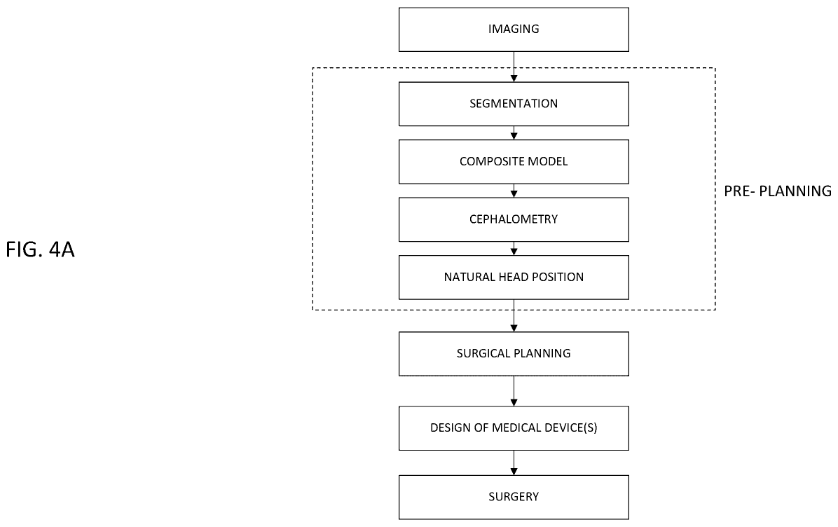

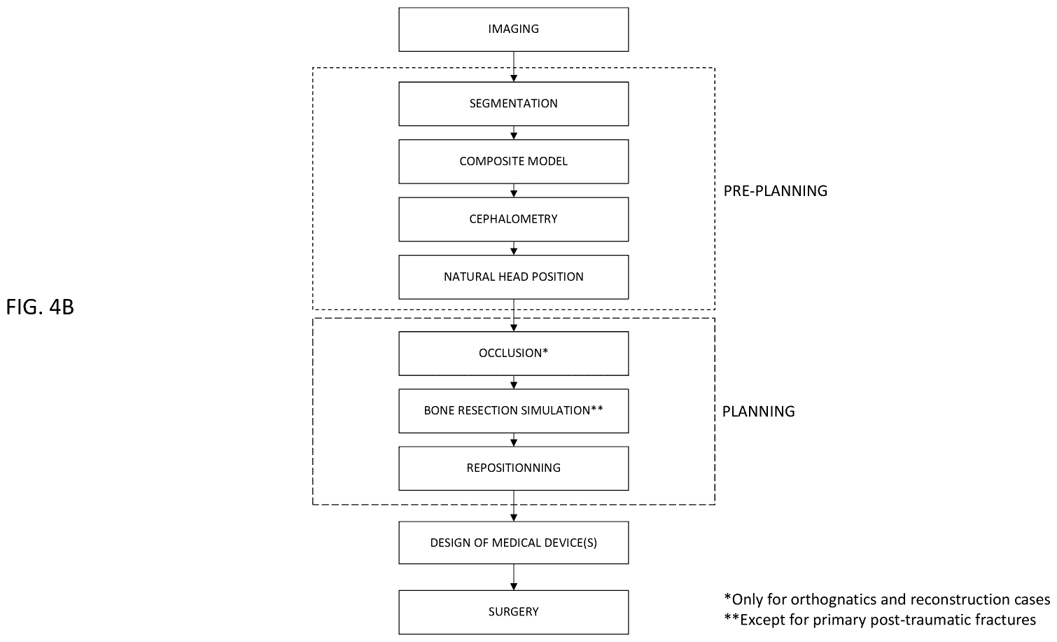

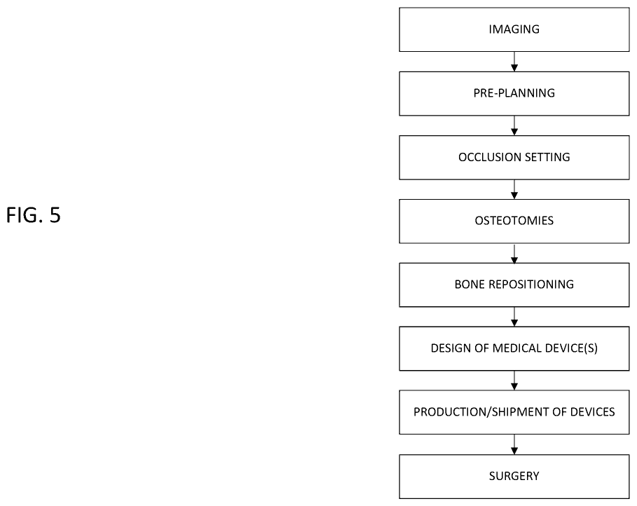

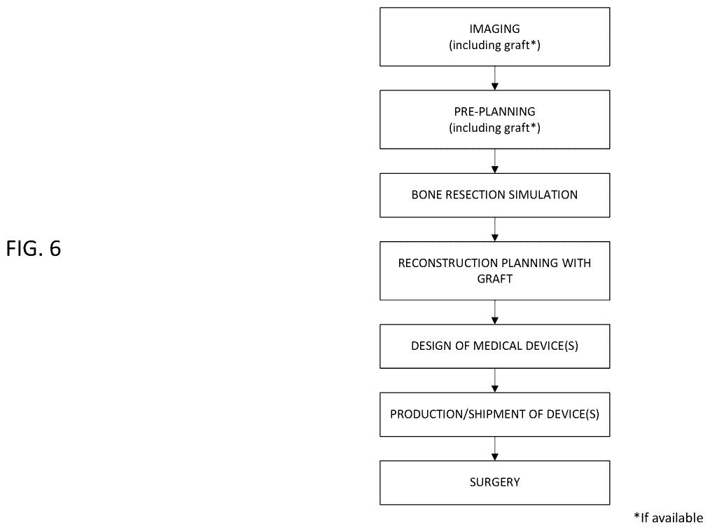

Some virtual planning techniques are illustrated by workflows depicted in FIGS. 4A, 4B, 5, and 6, according to certain embodiments.

Certain aspects of the novel systems may work with dedicated planning software, for example, the software marketed by the Belgian company Materialise under the name of Mimics and/or SurgiCase or the like such as PROPLAN CMF, and a user, typically a medically trained professional such as a surgeon, optionally assisted by a technician, may operate the system or one or more modules of the systems as described herein. A user may also be clinical engineer or a production engineer/technician or the like.

With computer-assisted surgery, e.g., surgical navigation or robotics, pre-operative imaging studies of the patient or medical images captured intra-operatively can be used. Alternatively, intra-operative 3-D surface meshes can be acquired with optical imaging to reconstruct the anatomy. The images can be displayed in the operating room (OR) on an external computer monitor and the patient's anatomy, e.g., landmarks, can be registered in relationship to the information displayed on the monitor. Since the surgical window is in a different location and has a different view coordinate system for the surgeon's eyes than the external computer monitor, hand-eye coordination can be challenging for the surgeon.

Alternatively, video-based AR (also known as video-see-through or video-pass-through) may also be used to allow the translation of the virtual surgical plan to the OR wherein the user is presented with an augmented video stream (e.g., in his/her headset or on a tablet) that overlays the virtual elements on a live feed captured with a display device (e.g., headset or a tablet). Further, a video see-through type of an AR system may be used in combination with an optical see-through system, e.g., by providing two separate optical systems or by operating a virtual video-see-through augmented environment in the optical see-through part of the system (e.g., on a Microsoft HoloLens). Aspects of mixed-reality systems, such as AR, provide interactive environments for the user. Some of the advantages associated with the use of an interactive system in the OR include reducing the time spent in the OR, real-time guided adaption of medical devices resulting in an overall satisfactory surgeon and/or patient experience, the adaptability of the system allowing the surgeon to deal with any complications encountered in the OR in an informed and efficient manner in real time, etc.

Unlike conventional methods, the systems and methods described herein provide improved ability for the surgeon to plan, visualize, and evaluate surgical procedures resulting in improved patient outcomes and/or operational efficiency gains for the physician (time, logistics, etc). Further, the systems and methods provide a virtual environment by providing access to relevant information at a dedicated location via a virtual workbench to the user thereby increasing the adaptability and efficiency of the system. Additionally, the systems and methods described herein provide the user access to operate other external systems that are integrated in the AR system network such as an additive-manufacturing device, such as 3-D printer to manufacture one or more components on the fly such as one or more medical devices (instruments, guides, implants, screws, plates, etc), anatomical models, other miscellaneous items that may be useful during surgery such as surgical tags, etc., robotic systems (or arms). The systems and methods provide improved accuracy in surgical procedures as compared to traditional systems, again improving patient outcomes and the field of medicine by providing a dedicated, one-stop, virtual workbench where all the information is available in an organized, user-friendly platform.

Aspects of the disclosure describe systems and methods that work with augmented-reality technology to provide a virtual (augmented) environment during a surgical procedure.

A virtual (augmented) environment comprises of a plurality of virtual (augmented) elements that are overlaid over a view of the user's real-world environment.

### AR System

Aspects of the present disclosure describe an AR system, its (modules) components and the interaction between one or more components when in use.

An example AR system comprises one or more of: an I/O module that provides interaction between the user and the system in the augmented environment, a (common) network, one or more external systems integrated in such a way that they can be operated via the AR system, a display unit comprising of one or more of display devices for displaying augmented data, a scanning-device and image-storage (database) module for operating medical imaging devices and storing their (scans) output and/or for storing and retrieving medical images, inventory, etc., a case management module for retrieving cases (plans) by a user, a virtual-3-D-model-creation module for generating 3-D virtual models of anatomical part(s) or medical devices and/or instruments, a planning module for planning one or more surgical steps, a visualization module for generating augmented data, a calibration module for calibrating one or more medical instruments, devices, patient, etc., a registration module for registering physical work objects so that they can be accessed in the AR environment, a guidance module for guiding a user during a surgical procedure, a controlling module for operating one or more external devices, a virtual workbench module for providing a user interface, etc. The different modules and their interaction will be described herein.

When blending an actual scene—i.e., reality—with a virtual environment, the AR system displays to the user a combined view of the actual scene and the virtual environment. This means that parts of the user's field of view may be occupied by a view of the actual scene, other parts may be occupied by renderings of elements from the virtual environment, and yet other parts may be occupied by a blend of both (e.g., virtual elements overlaid semi-transparently onto a view of the actual scene).

AR systems typically comprise at least one camera. This camera may be embedded in the device that also comprises the display unit (e.g., handheld device, head-mounted display), but may also be external to the display system (e.g., one or more wireless or wired cameras attached at specific location(s) in the operating room). Multiple camera systems may be used to circumvent line-of-sight issues or to address potential distortion between one of the cameras and the scene (e.g., when wearing a surgical mask over a head-mounted device). Different parts of the light spectrum, visible or not visible, may be acquired with different cameras, e.g., infrared imaging systems or visible-light cameras. Complex camera systems such as time-of-flight cameras or lidar may also be part of the AR system. The data coming from multiple cameras may be used independently or may be combined wherever possible.

In order to display such a combination of the actual scene and a virtual environment, an AR system comprises at least one display unit. Examples can include head-mounted display glasses, handheld devices, portable devices, and/or fixed devices, which either display or project elements of the virtual environment on an otherwise transparent lens or other objects in the actual scene, or comprise one or more cameras to record the actual scene and then display the blended scene on a monitor.

To be able to meaningfully combine the actual scene and the virtual environment in one image, AR systems typically need to bring both into a common coordinate system. One possibility is to register the virtual environment to the actual scene, such that both share a coordinate system, e.g., the world coordinate system. An example registration technique is discussed in U.S. Pat. No. 10,687,901, which is hereby incorporated by reference in its entirety. It should be understood that other suitable registration techniques may be used. For example, a user may manipulate the location of the virtual environment using hand gesture interactions provided by the AR system. A movement of the user's hands (or a controller or other interface element) will lead to a relative displacement of the virtual environment or of a virtual element of the virtual environment in relation to the physical environment.

The AR system then needs to determine the display unit's viewpoint and viewing direction, e.g., the display unit's coordinate system, in relation to the common coordinate system of the actual scene and virtual environment, effectively bringing the display unit into the same common coordinate system. Different systems and methods are known in the art to achieve this. For example, cameras in fixed positions or cameras/sensors embedded in the display unit may be used to track the movement of the display unit and deduce from this movement the display unit's coordinate system in relation to (parts of) the actual scene and/or the environment (e.g., simultaneous localization and tracking (SLAM)). SLAM technology is an extension of traditional tracking in which an application tries to recognize the physical world through feature points, to generate a dynamic map. The common coordinate system in which the actual scene, the virtual environment and the display unit are brought—or world coordinate system—can be the coordinate system of the actual scene, the coordinate system of the virtual environment, the coordinate system of the display unit or another coordinate system. It is also possible to bring everything into the coordinate system of a moving or movable object, such as an actual object in the actual scene.

To blend an element of the virtual environment into a view of the actual scene, the AR system may align a virtual camera with the display unit's coordinate system, create a rendering of the virtual element using the virtual camera, and combine the rendered image with the view of the actual scene in the display unit.

Embodiments of systems and methods described herein provide visual guidance/assistance during surgical procedures using AR technology.

Embodiments of systems and methods described herein provide a virtual workbench for providing a dedicated, virtual environment for designing, planning, operating, guiding and/or assistance during surgical procedures using AR technology.

In some embodiments, the systems and methods provide visual guidance/assistance to user(s) using an optical head-mounted display (OHMD) and/or overhead display. In some embodiments, the system uses a mobile or wearable device (e.g., smartphone, tablet, etc.) to provide such guidance.

According to an embodiment, the systems and methods provide visual guidance/assistance by an augmented-reality system during craniomaxillofacial (CMF) surgery, such as orthognathic surgery, reconstruction surgery, CMF trauma reconstruction (e.g., for bone trauma such as fractures of the zygoma, orbital floor, sinus, skull base, cranial vault, midface, nasal NOE, tooth, alveolar process, mandible, maxilla), CMF oncological reconstruction, CMF distraction surgery, CMF aesthetic reconstruction, craniofacial surgery (e.g., such as for craniosynostosis, congenital deformities, etc.) or placing temporo-mandibular joint-replacement or joint-resurfacing implants.

Although certain aspects of the description that follows describe embodiments of systems and methods being used for craniomaxillofacial surgery, the systems and methods can similarly be used during surgical procedures of non-CMF regions as well, such as pelvic/acetabular fracture surgery, placing spinal rods, placing spinal osteosynthesis and fusion plates, placing modular implant systems (e.g., placing joint-replacement or joint-resurfacing implants, such as knee, hip, shoulder, elbow, ankle or wrist replacement implants, such as lower extremity mega prostheses), forearm osteotomy (such as distal radius reconstruction or mid-shaft radius or ulna corrective osteotomies), veterinary osteosynthesis applications, placing extremity osteosynthesis plates (hand, foot, ankle), placing external fixators or cartilage-repair surgery. Parts of the system may also be used in non-skeletal surgeries, e.g., during minimally invasive procedures, pulmonary or cardiac or cardiovascular interventions, such as placement of stents, grafts, bypasses or valves, or valve repair.

Although certain aspects of the description that follows describe embodiments of systems and methods being used for craniomaxillofacial surgery, the systems and methods can similarly be used to replace or repair any bony anatomy, such as the skull, head, face and/or neck, joint, hip, knee, elbow, femur, jaw, shoulder, etc.

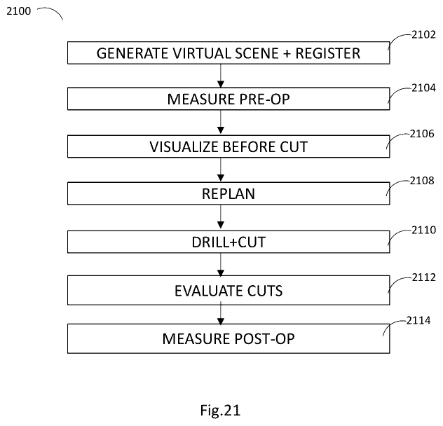

A workflow for augmented-reality-enhanced surgery according to certain embodiments is shown in FIG. 21, as is elaborated further below.

The systems and methods described herein may be implemented in a computing environment comprising one or more computing devices configured to provide various functionalities.

Certain embodiments described herein provide an AR system comprising one or more modules. Each module may be assigned a specific function, such as: the scanning-device and image-storage module may be configured to store a plurality of patient data, the planning module may be configured to plan one or more steps of virtual surgical planning, etc. One or more modules may be configured to work together to execute a specific task as described herein, such as for virtual surgical planning the scanning-device and image-storage module and the planning module may work together to create or modify a virtual surgical plan. It is to be understood that depending on the action/task to be executed, different combinations of modules may be configured to work together.

### Modules of the AR System

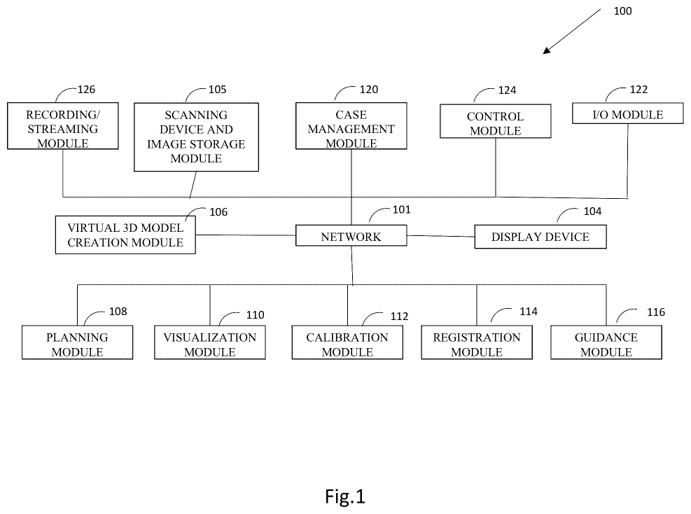

FIG. 1 is an example of a computer environment 100 suitable for implementing certain embodiments described herein.

### Network 101

The computer environment 100 may include a network 101. The network 101 may take various forms. For example, the network 101 may be a wired network, a wireless network or a combination of both. For example, the network 101 may be a local-area network installed at a surgical site. In some embodiments, the network 101 may be a wide-area network such as the Internet. In some embodiments, the network 101 may include a bus on a device itself. In other embodiments, the network 101 may be a combination of local-area networks, wide-area networks, and local buses. Typically, the network will allow for secured communications and data to be shared between various computing devices/components/modules. Each computing device/component/module may be a typical personal computer device that runs an off-the-shelf operating system such as Windows, Mac OS, Linux, Chrome OS, or some other operating system. Each computing device/component/module may have at least one application software installed to allow it to interact via the network 101 with other software(s) stored on various other modules and devices in the computing environment 100. This application software may take the form of a web browser capable of accessing a remote application service, for example via cloud computing. Alternatively, the application software may be a client application installed in the operating system of the computing device. Each computing device/component/module may also take the form of a specialized computer, specifically designed for medical surgical imaging and planning, or even more specifically for augmented reality. Each computing device/component/module may further take the form of a mobile device or tablet computer configured to communicate via the network 101 and further configured to run one or more software modules to allow a user to perform various methods described herein.

A number of modules and devices are shown coupled to network 101. Each of these modules/devices may be separate as shown and correspond to different computing devices (e.g., comprising a memory and a processor configured to execute the functions of the module). In certain aspects, the modules may be applications that run on a computing device. Though the devices and modules are shown as separate and communicating via network 101, different modules and devices may run on a same computing device, in any suitable combination of any suitable number of computing devices. The modules and devices may also be accessible via the virtual workbench at the physical location of the virtual workbench, as described herein.

### I/O Module 122

The computing environment 100 may include an Input/Output (I/O) module 122. The I/O module 122 may be configured to transfer data between one or more computing devices and one or more peripheral devices such as the display unit 104. The I/O module 122 may further provide ways for the user to interact with it to give instructions to the system, e.g. to activate a particular function or to change the location, orientation and/or scale of a displayed element, for example, by means of gesture-based controls. The I/O module 122 may also comprise physical input devices, such as pedals, pointing devices, buttons, keyboards, touch screens and the like. The I/O module 122 may also comprise one or more microphones for receiving voice-control instructions.

For example, imaging systems (such as a medical imaging device or imaging devices like microscopes), sensors, markers, additive-manufacturing device(s) and cameras may correspond to I/O module 122 of FIG. 1. Further, the computer device(s) may run the various modules described with respect to FIG. 1. Further, the display unit may correspond to the display device 104 of FIG. 1.

The I/O module 122 may also be used to access the virtual workbench as described herein.

Certain embodiments comprise methods of using I/O module 122 of the AR system before or during a surgical procedure as described herein.

Certain embodiments comprise methods wherein the I/O module 122 is used during a craniomaxillofacial surgical procedure as described herein.

Certain embodiments comprise methods wherein the I/O module 122 is used in combination with one or more modules of the AR system as described herein.

### Display Unit/Device(s) 104

The computing environment 100 includes a display device 104. The display device 104 may include one or more of an optical head-mounted display (OHMD), monitor, TV, overhead display, and/or mobile device, such as a tablet computer or smartphone, etc., used to display a virtual environment as part of a real environment. In certain aspects, such as where the display device 104 is a head-mounted display, the display device may include one or more accelerometers, cameras, positioning systems, etc., that track a position, location, orientation, etc., of the display device 104.

In certain aspects, the virtual workbench is accessible via a display device 104 such as an OHMD as described herein.

Certain embodiments comprise methods of using display device 104 of the AR system before or during a surgical procedure as described herein.

Certain embodiments comprise methods wherein the display device 104 is used during a craniomaxillofacial surgical procedure as described herein.

Certain embodiments comprise methods wherein the display device 104 is used in combination with one or more modules of the AR system as described herein.

Certain embodiments comprise methods of using the display device 104 for accessing the virtual workbench as described herein.

### Scanning-Device and Image-Storage (Database) Module 105

The computing environment 100 may further include a scanning-device and image-storage module 105. In certain aspects, the scanning-device and image-storage module 105 includes a large database designed to store image files captured by scanning-device and image-storage module 105. These images may be DICOM images, images or scans of medical devices, medical instruments, or other types of images. The scanning-device and image-storage module 105 may also be a standalone database, for example in a server-based system, such as a PACS system, having dedicated storage optimized for medical image data. Optionally, the standalone database may have dedicated storage optimized for the creation of an inventory of medical devices. The scanning-device and image-storage module 105 may alternatively or additionally comprise a medical imaging device which is configured to scan a patient to create images of their anatomy. Additionally, various surgical approaches may also be stored in the database. In the computing environment 100 shown in FIG. 1, the scanning-device and image-storage module 105 may comprise a dental scanner, facial scanner, optical scanner, X-ray machine, CT scanner, CBCT scanner, ultrasound device, camera, or MRI device. However, a skilled artisan will appreciate that other scanning technologies may be implemented which provide imaging data that can be used to create three-dimensional anatomical models.

In certain aspects, scanning-device and image-storage module 105 comprises a storage configured to store images generated outside of computing environment 100 and/or generated by scanning-device and image-storage module 105. Accordingly, scanning-device and image-storage module 105 may include both a scanning device and an image storage, or only one of a scanning device or an image storage.

Patient data may also comprise one or more of medical images, personal information, such as age, sex, weight, height, ethnicity, lifestyle, activity level, medical history, any data gathered during pre-surgical exams, such as complaints, pain scores, fractures, dental measurements, information about degenerative or congenital defects, trauma or oncology-related information, any data gathered intra-operatively, such as anatomical or functional measurements, and others.

Stored (patient) data may also comprise information such as cephalometric landmarks and/or analysis information (e.g., manual or automated identification of landmarks on X-rays or (CB)CT images of the patient serving as input for manual or (e.g., semi-)automated cephalometric measurement calculations). Data may further comprise a digital representation of patient dentition (such as intra-oral scans, optical scans of plaster casts made from dental impressions, etc.). Digital scans may include detailed scans of one or more of the maxillary teeth and one or more of the mandibular teeth, including information relating to teeth characteristics such as presence, absence, chipped surface, coloration, etc. Optionally, an occlusion scan may also be acquired or alternatively the occlusion can be set virtually by means of manual or (e.g., semi-)automated software tools. Data may further comprise information regarding a patient's jaw movements wherein the user tracks the patient's jaw, effectively measuring a relative displacement between maxilla and mandible simulating for example the position of the mandible with the condylar heads in centric relation, or a chewing motion or the opening/closing of the mouth instead of passively during surgery for more correct information. This input may also be provided from patient measurements done pre-operatively with a jaw-registration system like the Zebris system (product of Zebris Medical GmbH™).

Apart from patient data, a library (inventory) of medical devices (such as implants, guides, plates, etc.), medical instruments (such as screws, drill bits, bending pliers, etc.) in the form of lists or images or virtual three-dimensional models (e.g., as created by the virtual-3-D-model-creation module 106 as described herein) may also be stored. In certain embodiments, patient data and/or data from an inventory (also known as a library) of medical devices may be loaded from a file, a storage medium, cloud database, scanned using a digital recognition device such as a camera or barcode/QR code scanner or entered manually into the scanning-device and image-storage module 105 in the OR.

As described herein, the collected data may be used to generate a pre-operative virtual model of a part of the patient's anatomy, such as a bone structure, such as a skull or a portion thereof, in three dimensions. This constitutes the pre-operative shape of the bone. This shape may then be modified to produce a modified virtual model corresponding to the planned post-operative shape of the bone, in three dimensions. Any of the three-dimensional models may be annotated or marked to indicate anatomical landmarks such as points, planes, lines, curves, surfaces that may be useful during planning. The collected data is also stored in the scanning-device and image-storage module 105.

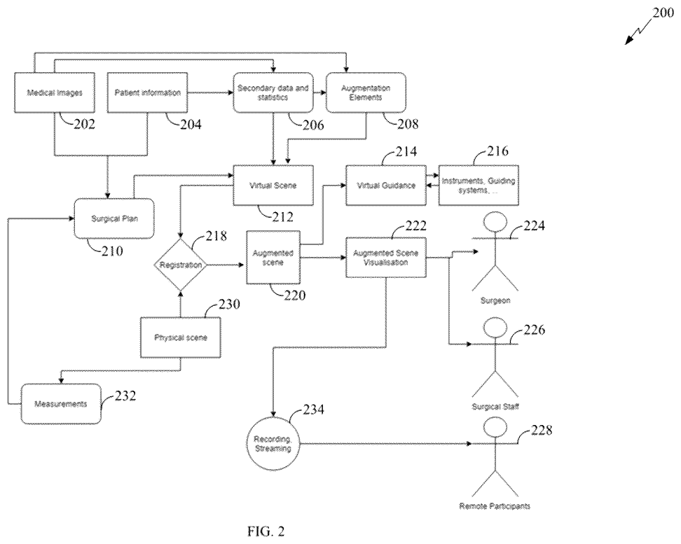

Input data comprising patient information 204 is processed to construct/design a surgical plan. For example, as shown in workflow 200 of FIG. 2, medical images 202 and patient information 204 may be used to generate a surgical plan 210. In certain aspects, surgical plans may be stored in the scanning device and image storage module 105.

In certain aspects, the scanning-device and image-storage module 105 is also accessible at the virtual workbench, as described herein.

Certain embodiments comprise systems and methods of using scanning-device and image-storage module 105 of the AR system before or during a surgical procedure as described herein.

Certain embodiments comprise methods wherein the scanning-device and image-storage module 105 is used during a craniomaxillofacial surgical procedure as described herein.

Certain embodiments comprise methods wherein the scanning-device and image-storage module 105 is used in combination with one or more modules of the AR system as described herein.

Certain embodiments comprise methods of accessing the scanning-device and image-storage module 105 at the virtual workbench as described herein.

Certain embodiments comprising methods of retrieving patient data for a craniomaxillofacial surgery are described herein.

Certain embodiments comprising methods of retrieving patient data for use during an orthognathic surgery, are described herein.

Certain embodiments comprising methods of retrieving patient data for use during a mandible reconstruction surgery, are described herein.

Certain embodiments comprising methods of retrieving patient data for use during a maxilla reconstruction surgery, are described herein.

Certain embodiments comprising methods of retrieving patient data for use during an orbital-floor reconstruction surgery, are described herein.

Certain embodiments comprising methods of retrieving patient data for use during a tumor-resection and reconstruction surgery of one or more anatomical parts of the CMF region, are described herein.

Certain embodiments comprising methods of retrieving patient data for use during a trauma surgery of one or more anatomical parts of the CMF region, are described herein.

Certain embodiments comprising methods of retrieving patient data for use during a craniosynostosis surgery, are described herein.

### Case-Management Module 120

The computing environment 100 may also include a case-management module 120 for retrieval of previously planned cases.

A case-management module 120 may allow the user to access and visualize a list of one or more cases that each represent a surgical plan and/or virtual 3-D model of a patient. The case-management module 120 may retrieve its data from the scanning-device and image-storage module 105 (e.g., via a cloud or local database). The AR system itself or the case-management module 120 as part of the AR system may request the user to provide their credentials, e.g., in the form of a login and password, pin code or through biometric recognition such as iris recognition. The user may be able to navigate a case list in the AR system to open one or more cases in the AR system. Additionally, the case-management module 120 may be accessed at the virtual workbench. The case-management module 120 may allow the user to create local copies of (part of) cases to access these when the AR system is not connected to a network (for offline continuity). The case-management module 120 may visibly distinguish between cases which are available offline and cases which are not in its interface. The case-management module 120 may provide the user with information about a patient such as the patient's name, unique patient identifier, surgery type, treating physician, surgery date, age, gender, etc. The case-management module 120 may also provide the user with information about the status of the case, for example whether the case is being processed by an engineer, or whether it is finished. The status of medical devices linked to the case (e.g., implants or guides) may also be provided to the user.

Certain embodiments comprise methods of using case-management module 120 of the AR system before or during a surgical procedure as described herein.

Certain embodiments comprise methods wherein the case-management module 120 is used during a craniomaxillofacial surgical procedure as described herein.

Certain embodiments comprise methods wherein the case-management module 120 is used in combination with one or more modules of the AR system as described herein.

Certain embodiments comprise methods of accessing the case-management module 120 at the virtual workbench as described herein.

Certain embodiments comprising methods of retrieving surgical plan for a craniomaxillofacial surgery are described herein.

Certain embodiments comprising methods of retrieving an orthognathic plan (performed on a mandible) from the case-management module 120 are described herein.

Certain embodiments comprising methods of retrieving an orthognathic plan (performed on a maxilla) from the case-management module 120 are described herein.

Certain embodiments comprising methods of retrieving an orthognathic plan (performed both on a mandible and maxilla) from the case management-module 120 are described herein.

Certain embodiments comprising methods of retrieving an orbital-floor reconstruction plan from the case-management module 120 are described herein.

Certain embodiments comprising methods of retrieving a craniosynostosis plan from the case-management module 120 are described herein. Certain embodiments comprising methods of retrieving a tumor-resection plan from the case-management module 120 are described herein.

### Virtual-3-D-Model-Creation Module 106

The computing environment 100 may also include a virtual-3-D-model-creation module 106. The virtual-3-D-model-creation module 106 may take the form of computer software, hardware, or a combination of both which retrieves the medical imaging data from scanning-device and image-storage module 105 and generates one or more virtual three-dimensional models, e.g., of one or more anatomy parts, such as by using stacks of 2-D image data or point-cloud scans. Alternatively, the virtual-3-D-model-creation module 106 may also retrieve images or point-cloud scans of medical devices (such as implants, instruments, or guides) and generate virtual three-dimensional models. The virtual-3-D-model-creation module 106 may be or may comprise a commercially available image-processing software for three-dimensional design and modelling such as Mimics. However, other image-processing software may be used. In some embodiments, the virtual-3-D-model-creation module 106 may be provided via a web-based network application that is accessed by a computer over the network. Alternatively, the virtual-3-D-model-creation module 106 may be a software application that is installed directly on a computing device, and accesses scanning-device and image-storage module 105 via the network 101. In general, the virtual-3-D-model-creation module 106 may be any combination of software and/or hardware located within the computing environment 100 which provides image-processing capabilities on the image data stored within the scanning-device and image-storage module 105.

Data stored in the scanning-device and image-storage module 105 may be retrieved by the virtual-3-D-model-creation module 106 during a virtual surgical-planning session. For example, medical images 202, such as X-ray, CT, CBCT, MRI, ultrasound images, or dental images from scanning-device and image-storage module 105 may be converted through segmentation by virtual-3-D-model-creation module 106 into corresponding one or more virtual 3-D models of one or more anatomy parts, such as bony anatomy, cartilage, organs, organ walls, vasculature, nerves, muscles, tendons and ligaments, blood pool volume, teeth, tooth roots, etc. and/or possible pre-existing hardware, such as any implants placed prior to acquiring medical images 202, e.g., dental implants, tooth crowns or bridges or tooth fillings, orthopaedic or craniomaxillofacial implants, osteosynthesis implants, prosthetic implants, joint-arthroplasty implants, stents, prosthetic heart valves and the like. The segmentation may be performed using known techniques, such as computer-implemented graph-portioning methods, fast-marching methods, region-growing methods, edge detection, etc. Additionally or alternatively, virtual 3-D models may be obtained by virtual-3-D-model-creation module 106 reconstructing a 3-D shape based on one or more 2-D images (e.g., X-ray, ultrasound) from scanning-device and image-storage module 105, using prior population knowledge (e.g., by using Statistical Shape Models (SSMs)) or by directly measuring on the patient's exposed anatomy using marking and/or motion-tracking devices, such as provided by I/O module 122 (e.g., which may be coupled to cameras, motion tracking devices, etc.) or surface-scanning methods provided by scanning-device and image-storage module 105. The virtual 3-D models may be iteratively improved by virtual-3-D-model-creation module 106 and presented on display device 104 to the user as more information becomes available, e.g., while performing measurements intra-operatively. Additionally, virtual 3-D models of one or more medical devices, such as plates, instruments, guides, screws, etc. (herein after also referred to as virtual medical-device 3-D model) may be obtained by virtual-3-D-model-creation module 106 reconstructing a 3-D shape based on 2-D image(s) from scanning-device and image-storage module 105 or by directly scanning a medical device using optical scanning devices, marking devices and/or shape-recognition techniques.

In certain aspects, the virtual 3-D models may be created before surgery (using pre-operative imaging) or during surgery (using intra-operative information or a mix of pre-operative and intra-operative information). Same can be done for medical devices as well wherein generic virtual 3-D models of standard medical devices (e.g., implants, guides, etc.) are created using the virtual-3-D-model-creation module 106 and stored in scanning-device and image-storage module 105.

Anatomical landmarks may be determined manually by indicating them on the medical images or on the virtual 3-D models (herein after also referred to as virtual anatomical 3-D models) using I/O module 122, automatically using feature-recognition techniques or by fitting statistical models that comprise information on the anatomical landmarks on the medical images or on the virtual 3-D models, by intra-operative annotation of the landmarks, using motion-tracking-based reconstruction (e.g., by contacting the actual patient anatomy with a motion-tracked stylus) to derive such landmarks (e.g., orbital floor) or by fitting statistical models to intra-operatively obtained 3-D scans of the anatomy. Anatomical coordinate systems, anatomical axes and/or mechanical axes may be derived from the anatomical landmarks.

Data may further comprise data derived from devices such as one or more radiography studies or tomodensimetric scanner sections, CT, (CB)CT, MRI, PET, or ultrasound, surface reconstructions obtained using optical scanners, dental scans and other devices used to acquire facial information. Information such as cephalometric landmarks and analyses associated with the landmarks such as measurements may also be obtained (e.g., through manual or automated identification of landmarks on X-rays or (CB)CT images of the patient serving as input for manual or (e.g., semi-)automated cephalometric measurement calculations). Data may further comprise digital representation of patient dentition (such as intra-oral scans, optical scans of plaster casts made from dental impressions, etc.). Digital scans may include detailed scans of one or more of the maxillary teeth and/or one or more of the mandibular teeth, including information relating to teeth characteristics such as presence, absence, chipped surface, coloration, partial presence (or absence), etc. Optionally, an occlusion scan may also be acquired or alternatively the occlusion can be set virtually by means of manual or (e.g., semi-)automated software tools. These imaging data are then processed on a computer such as using a specific application (such as Mimics) to generate a three-dimensional reconstruction of the images. For example, this stage may comprise accessing data indicative of a pre-operative maxillofacial anatomy of a patient and generating a virtual three-dimensional model of said anatomy using said data. Additionally, or alternatively, data from different sources, e.g., different image modalities, and/or virtual 3-D models may be registered onto each other to create mixed-modality virtual models or enhanced virtual 3-D models, e.g., by using any suitable registration techniques known in the art, such as image-registration techniques, surface-registration techniques or point-set-registration techniques, e.g., the iterative closest-point technique. For example, the digital scans and/or virtual 3-D models of the dentition may be registered with medical imaging data, such as CT, MRI or radiographic image data, or with virtual 3-D anatomical models obtained from medical imaging, e.g., radiography, studies. Data may further comprise information regarding a patient's jaw movements wherein the user tracks the patient's jaw, effectively measuring a relative displacement between maxilla and mandible simulating for example the position of the mandible with the condylar heads in centric relation, or a chewing motion or the opening/closing of the mouth instead of passively during surgery for more correct information. This input maybe also be provided from patient measurements done pre-operatively with jaw registration system like the Zebris system (product of Zebris Medical GmbH™). The data regarding patient's jaw movement may then be used by the virtual-3-D-model-creation module 106 to generate a three-dimensional reconstruction of the jaw. Additionally, an animated virtual 3-D model of the patient jaw may be created by the virtual-3-D-model-creation module 106 to simulate the opening/closing of the mouth.

Certain embodiments comprise methods of using virtual-3-D-model-creation module 106 of the AR system before or during a surgical procedure as described herein.

Certain embodiments comprise methods wherein the virtual-3-D-model-creation module 106 is used during a craniomaxillofacial surgical procedure as described herein.

Certain embodiments comprise methods wherein the virtual-3-D-model-creation module 106 is used in combination with one or more modules of the AR system as described herein.

Certain embodiments comprise methods of accessing the virtual-3-D-model-creation module 106 at the virtual workbench as described herein.

In certain embodiments, the virtual-3-D-model-creation module 106 may be used for adapting a virtual model before or during a surgical procedure as described herein. In certain aspects, the virtual-3-D-model-creation module 106 may be used with planning module 108 for adapting a virtual model as described herein.

Certain embodiments comprising methods of creating a virtual 3-D model for use during a craniomaxillofacial surgery, are described herein.

Certain embodiments comprising methods of creating a virtual 3-D model for use during an orthognathic surgery, are described herein.

Certain embodiments comprising methods of creating a virtual 3-D model for use during a mandible reconstruction surgery, are described herein.

Certain embodiments comprising methods of creating a virtual 3-D model for use during a maxilla reconstruction surgery, are described herein.

Certain embodiments comprising methods of creating a virtual 3-D model for use during an orbital-floor reconstruction surgery, are described herein.

Certain embodiments comprising methods of creating a virtual 3-D model for use during a tumor-resection and reconstruction surgery of one or more anatomical parts of the CMF region, are described herein.

Certain embodiments comprising methods of creating a virtual 3-D model for use during a trauma surgery of one or more anatomical parts of the CMF region, are described herein.

Certain embodiments comprising methods of creating a virtual 3-D model for use during a craniosynostosis surgery, are described herein.

### Planning Module 108

The computing environment 100 may also include a planning module 108. The planning module 108 may be configured to perform surgical planning for an AR system.

A virtual surgical plan is created during a virtual-surgical-planning session using imaging data to accurately plan a surgical procedure in a computer environment. Conventionally, this is done on a standalone desktop system which is located outside the OR. With the AR system, a user may create a virtual surgical plan either pre-operatively (herein after also referred to as pre-op) or intra-operatively (herein after also referred to as intra-op) using the planning module 108.

In some cases, pre-operative planning of any osteotomy, resection or repositioning operations to be carried out for various bone fragments or portions is desirable in order to define a new position of the bone fragments or portions, so as to simulate and predict surgery outcome. This is translated into a virtual surgical plan. A virtual surgical plan is then translated to the patient via intra-operative guidance techniques such as computer navigation, (patient-specific) guides, (patient-specific) implants, (patient-specific) (pre- or post-planning) anatomical models or robotics or a combination thereof. Alternatively, guidance may be provided for planning a surgery intra-op and transferring the plan to a patient by the guidance module 116 as described herein.

During planning, the surgeon prepares for the surgery before a surgical procedure. The steps comprise one or more of: gathering patient data, patient diagnosis, analyzing the anatomy to determine the defects, such as fractures, predicting soft-tissue balance and range of motion after surgery based on the defects and the pre-op range of motion, determining the procedure to follow (e.g., orthognathic surgery, mandible reconstruction, maxilla reconstruction, cranial reconstruction, midface reconstruction, orbital-floor reconstruction) and the associated surgical approach to be used, preselecting the instruments and the implants (e.g., implant type, size, length), determining the positions of implants and fixation elements such as screws, nails or pegs (e.g., type, orientation, length, location), and any associated osteotomy or resection locations, amongst other surgical parameters. These steps can be performed for a plurality of implants. All of the mentioned information about a patient may be stored in the scanning-device and image-storage module 105.

Anatomical landmarks, any derived coordinate systems and/or axes, and/or 3-D anatomical shape data may be used by planning module 108 to determine (e.g., the most) suitable implant components (e.g., prosthetic devices, screws), their sizes and their positions (e.g., locations and orientations) in relation to said data. For example, a user may browse through a series of virtual implants such as plates, position these on the virtual (or real) anatomy and, based on a visual assessment, determine the implant to use. Embodiments of planning module 108 of AR systems herein may allow the user to design patient-specific medical devices, such as patient-specific guides to be used during surgery for guiding certain surgical steps, or patient-specific implants, e.g., for fixating bones or portions. Virtual 3-D models of these patient-specific devices in their intended relative position with respect to the patient anatomy may be part of the surgical plan. The designs of such patient-specific devices may be transferred, e.g., via I/O module 122 or control module 124, to a peripheral manufacturing device, such as a 3-D printer. The surgical plan 210 may be updated by planning module 108 during surgery when additional information (such as complications that could not be predicted pre-operatively) becomes available. During planning, the surgeon may choose the landmarks that he/she wishes to be highlighted and overlaid on the patient during surgery by the display device 104.

Embodiments of planning module 108 of AR systems herein may allow both pre-operative and intra-operative planning. Intra-operatively, the user may use the planning module 108 to plan the surgery before the patient is opened or after the patient has been opened to reveal the surgical site, or to adapt a pre-operatively created plan.

In certain instances, the user may use the planning module 108 to plan the surgery on a combination of one or more physical 3-D models and one or more virtual 3-D models. For example, the AR system may provide guidance by overlaying a rendered image of one or more virtual 3-D models onto a view of one or more physical 3-D models. For example, the user may plan the surgery on an anatomical 3-D patient-specific model such as a 3-D-printed patient-specific model and/or/in combination with a virtual 3-D model of a medical device such as a standard medical device (e.g., implant). Additionally, or alternatively, the user may use the planning module 108 to plan one or more surgical steps using a physical template (e.g., a template or replica that may be provided by the manufacturer of a standard device or 3-D printed) of a medical device. A physical template may be used in combination with a virtual 3-D model of a medical device. Additionally, or alternatively, the AR system may provide additional guidance by overlaying a view of the physical template with visual indicators (e.g., by showing arrows, lines, planes, points, etc.) as described in certain embodiments herein.

In certain instances, the surgical plan 210 may be created or adapted fully automatically by planning module 108 and without any user interaction, e.g., based on predictive algorithms or based on pre-defined rules optionally including surgeon-specific preferences. In certain instances, the surgical plan 210 may need to be created or adapted during surgery. The user, such as a surgeon, may plan the surgery before or after opening the patient.

In certain instances, the surgical plan 210 may need to be created or adapted during surgery by planning module 108. Embodiments of planning module 108 of AR systems herein may be used to modify the surgical plan 210 in an informed and efficient way. The planning module 108 of an AR system may require a plan to be transferred from another location (cloud, other workstation, different software, such as over network 101). This may be performed directly through a network link between the different systems. Alternatively, one or more visual references may be used (e.g., barcodes, QR codes) that either encode the planning information directly in them or encode a link that allows to transfer the information from said alternative location. Optical elements in the AR system, such as in display device 104 (e.g., the camera in a head-mounted device), may be used to capture the one or more visual references. The AR system may decode the one or more visual references.

In certain instances, the user may perform surgical planning intra-operatively by using virtual components and overlaying them on the actual or virtual patient's anatomy using the AR system. For example, the user may browse through several virtual implants stored in the scanning-device and image-storage module 105, e.g., in different sizes or shapes, during this process. This may allow the user to avoid having to try multiple implants in the real world, which would require them to be re-sterilized.

In certain embodiments, it is possible to perform intra-operative surgical planning without the need for pre-surgical planning or image acquisition. For example, in some embodiments, to plan a proximal cut on the mandible for an orthognathic surgery, the I/O module 122 and/or display device 104 may ask the user to manually indicate the mandible, for example by hand gestures or by using a stylus that is tracked by the system, and any fractures or anatomical landmarks or the like. From these landmarks, the planning module 108 can then derive the anatomical axis of the temporo-mandibular joint (TMJ). In some embodiments, to plan the cuts on the maxilla, the I/O module 122 and/or display device 104 may ask the user to manually indicate the maxilla and/or reference points defining the cutting planes and/or anatomical landmarks to serve as input for an algorithm determining the cutting planes. The I/O module 122 and/or display device 104 may also ask the user to move the patient's jaw, effectively performing a relative displacement between maxilla and mandible simulating for example the position of the mandible with the condylar heads in centric relation, or a chewing motion or the opening/closing of the mouth. By tracking that movement, the I/O module 122, or planning module 108, may be able to determine the rotation center of the TMJ, or the occlusion of the teeth. From this occlusion and/or rotation center and/or the manually indicated landmarks, the planning module 108 may then determine the mechanical axis of the TMJ. For example, once the appropriate point of rotation is determined at the TMJ, different axes of rotation (e.g., sagittal, coronal, etc.) can be used to optimally align the bone segments and minimize interferences. Other landmarks can be used for such procedures.

In certain embodiments, it may be possible to adapt a surgical plan or plan interactively based on intra-operative information such as in trauma and oncology cases. This may include clinical judgement and experience of the surgeon or may involve more elaborate objective analysis methods (such as characterization of patient's soft-tissue properties e.g., elasticity), data measured using the AR system itself (such as anatomical landmarks or occlusion) or data integrated from external systems such as force/stress/strain/loading sensors or robotic systems. The plan 210 may also be updated based on secondary data which is derived from measurements 232, for example by estimation of the load-bearing capacity or phonetic or masticatory estimation of a jaw through musculoskeletal modelling based on the primary measurements. Another example is to assess alignment of the dental midline (i.e., the line between the two maxillary central incisal teeth and the two mandibular central incisal teeth) with the middle of the face, thereby allowing to correct the plan in case of a deviated midline.

Adapting or creating the plan intra-operatively may be performed by directly controlling clinical parameters (such as implant type and size, implant position and rotation, or occlusion), using any of the mentioned interaction methods. The plan adaptation may be performed indirectly, e.g., through a cloud service, which may run in a virtual window in the augmented environment and will update the augmentation elements in a second stage or may be done directly on the augmentation elements as described herein.

The planning module 108 can also suggest multiple options for correcting a defect that would appear to require further correction. This may include the method and amount of ligaments releases, including the ligament to release such as in the case of TMJ reconstruction cases wherein it may prevent mandibular dislocation. This may also include the necessary bony recuts, either on mandible and temporal bone, and the amount of bone to be removed to create a stable joint while taking soft tissue into account, e.g., facial soft tissue components such as lips.

The AR system may enable the user to make informed decisions based on bony and soft-tissue data by presenting on display device 104 such data using visualization module 110 in an interactive format as will be described herein. During intra-operative planning, all the relevant information may be overlaid on the patient, patient-specific anatomical 3-D model or on one or more display monitors using display device 104. To guide and/or assist the surgeon in making informed decisions, the planning module 108 and/or visualization module 110 may highlight relevant information and communicate it to the surgeon using one of many visualization methods, some of which are elaborated below, subject to surgeon preference. Alternatively, the user (e.g., surgeon) may be presented with a blank canvas to plan the surgery intra-op (e.g., at the virtual workbench).

The augmented environment provided by certain embodiments herein may thus comprise different components such as one or more of: various augmentation elements (e.g., virtual anatomical models), virtual guidance tools (e.g., virtual drilling, cutting or reaming trajectories), visualization methods comprising display components, etc. One or more combinations are possible, subject to user preference. One or more augmented environments may be created or used by the AR system, depending on the number of users using the AR system. This makes the AR system customizable and user friendly such that multiple users can use the AR system at the same time and only deal with information that is individually relevant for them.

Certain embodiments comprising methods of using planning module 108 of the AR system before or during a surgical procedure are described herein.

Certain embodiments comprise methods wherein the planning module 108 is used during a craniomaxillofacial surgical procedure as described herein.

Certain embodiments comprise methods wherein the planning module 108 is used in combination with one or more modules of the AR system as described herein.

Certain embodiments comprise methods of accessing the planning module 108 at the virtual workbench as described herein.

Certain embodiments comprising methods of using the planning module 108 for planning a pre-op plan for an orthognathic surgery are described herein.

Certain embodiments comprising methods of using the planning module 108 for creating an intra-op plan for an orthognathic surgery are described herein.

Certain embodiments comprising methods of using the planning module 108 for creating a pre-op plan for a trauma surgery are described herein.

Certain embodiments comprising methods of using the planning module 108 for creating an intra-op plan for a trauma surgery are described herein.

Certain embodiments comprising methods of using the planning module 108 for creating a pre-op plan for a mandible reconstruction surgery are described herein.

Certain embodiments comprising methods of using the planning module 108 for creating an intra-op plan for a mandible reconstruction surgery are described herein.

Certain embodiments comprising methods of using the planning module 108 for creating a pre-op plan for a maxilla reconstruction surgery are described herein.

Certain embodiments comprising methods of using the planning module 108 for creating an intra-op plan for a maxilla reconstruction surgery are described herein.

Certain embodiments comprising methods of using the planning module 108 for creating a pre-op plan for an orbital-floor reconstruction surgery, are described herein.

Certain embodiments comprising methods of using the planning module 108 for creating an intra-op plan for an orbital-floor reconstruction surgery, are described herein.

Certain embodiments comprising methods of using the planning module 108 for creating a pre-op plan for a tumor-resection and reconstruction surgery of one or more anatomical parts of the CMF region are described herein.

Certain embodiments comprising methods of using the planning module 108 for creating an intra-op plan for a tumor-resection and reconstruction surgery of one or more anatomical parts of the CMF region are described herein.

Certain embodiments comprising methods of using the planning module 108 for creating a pre-op plan for a trauma surgery of one or more anatomical parts of the CMF region are described herein.

Certain embodiments comprising methods of using the planning module 108 for creating an intra-op plan for a trauma surgery of one or more anatomical parts of the CMF region are described herein.

Certain embodiments comprising methods of using the planning module 108 for creating a pre-op plan for a craniosynostosis surgery are described herein.

Certain embodiments comprising methods of using the planning module 108 for creating an intra-op plan for a craniosynostosis surgery are described herein.

### Visualization Module 110

The computing environment 100 may further include a visualization module 110. In certain embodiments, the visualization module 110 is configured to perform visualization, e.g., to provide virtual guidance, such as one or more of overlaying patient information, providing step-by-step guidance during the procedure, indicating any relevant anatomical landmarks, instruments, devices, best practices, etc. for executing the surgery.

During surgery, an augmented environment/scene 220 is created by visualization module 110 that may be wholly or partly visualized to the surgeon 224, his staff 226 and/or remote participants 228 such as through one or more display devices 104. This augmented environment 220 contains information that may guide/assist the surgeon during a procedure.

The actual/physical scene 230—i.e., reality—may be augmented by visualization module 110 with visualizations of virtual/augmentation elements 208 that are displayed on or around the patient, e.g., with an overlay on the anatomy, that may be partially transparent or fully opaque. In this way, parts of the surgical environment, for instance the patient anatomy may be highlighted, obscured, annotated, augmented, etc.

To allow the surgeon to focus on the area of interest, one or more obscuring objects such as hands, tools, implants, etc. may be hidden/made transparent, e.g., by using additional camera streams or historical camera data from I/O module 122 to replace the obscured part of the scene using augmented elements 208.

In some embodiments, the actual scene 230 may be augmented with visualization of virtual elements 208 that are spatially attached to the patient and his/her position (or the position of individual anatomical parts or landmarks of the patient) but don't necessarily overlap with the actual anatomy, thereby forming a virtual scene 212. This may include one or more of derived landmarks, annotations, lines, planes, zones, surfaces, dynamic information, target positions for guidance, etc., such as based on secondary data and statistics 206.

The actual scene 230 may also be augmented with visualization of virtual elements 208 on top and/or spatially attached to physical objects other than the patient in the operating room (OR). This may include one or more of instrumentation, medical devices, medical device components, navigation systems, robotic elements, furniture, members of staff etc.

The actual scene 230 may also be augmented with visualization of virtual elements 208 around the patient, either in a fixed location in the field-of-view of the surgeon and/or on dedicated virtual places corresponding to specific places in the real OR, such as a virtual workbench.

The virtual information that augments the environment may be static, moving along with one or more physical objects in the actual scene, moving independently of the actual scene, or floating freely in the augmented environment and movable by the user, as per surgeon convenience. Additionally, the virtual information may be updated in real time.

The virtual information may be adapted to account for visual obstruction of those virtual objects by the physical objects. For example, the system may use any known 3-D-scanning or stereophotogrammetry techniques to determine the three-dimensional shapes and positions of all objects within the user's field of view. Those shapes can then be added to the virtual environment and used to determine which parts of the virtual objects to be displayed are obscured by the objects from the actual scene. Based on this information, the visualization module 110 may omit those parts during rendering or render them differently, e.g., with a different transparency, luminosity, hue or saturation. Alternatively, or additionally, the visualization module 110 may add any of the virtual 3-D models of anatomical parts and/or medical devices obtained from medical images as described above to the virtual environment 212 and use those to determine visual obstruction. When multiple users are using the system, the above may be tailored to the individual user role, preference or physical position in the actual scene.

In certain embodiments, the visualization may be adapted based on real-time information that is captured during surgery, or through simulations or analyses that are performed based on this real-time information. The adaptation of the visualization may involve changing the data which is visualized (e.g., updating landmarks or anatomy) or changing the way data is visualized (e.g., changing transparency or color). For example, the user may use a pointer tracked in the AR system to capture certain landmarks which are visualized in the AR system. In another example, the user may perform certain measurements which would provide a safe zone for performing an osteotomy, e.g., a zone in which the osteotomy may be performed without risk of damaging delicate anatomical features, such as nerves, blood vessels or organs. The safe zone may be visualized in the AR system and may be modified if new data is acquired with the AR system. The virtual information may thus be dependent on elements that are visible in the scene, e.g., the real-time position of an implant, and be updated accordingly, e.g., by providing automatically updated optimal shaping of the implant in this position based on previous shaping of a chosen standard medical device (such as standard implant or plate), by providing automatically calculated optimal screw positions for that specific implant position or by highlighting the optimal implant position prior to fixation. For example, an implant such as a plate may be tracked relative to the patient's anatomy, e.g., while moving the plate over the mandible during orthognathic surgery. Based on the position of the plate, a suitable bending of the plate which aligns with the patient's anatomy may be determined. The AR system may perform these computations and provide the user with a proposal for a plate bend based on the live position on the anatomy.

In some embodiments, information about planned or simulated implant system components or component locations may also be visualized by visualization module 110 on display device 104 as part of the augmented environment 220. This may include screws, implants for maxilla, mandible, orbit, teeth, zygoma, skull, TMJ, etc. These may be displayed statically or dynamically, e.g., through the actual range of motion (opening/closing of the mouth) or via a simulation of the range of motion or via a robotic system. They may be used for visual analysis of the surgical plan, e.g., to evaluate sizing or screw locations. They may be overlaid before or after making cuts, the former, e.g., by obscuring the bone to be removed virtually. The information may be updated based on information in the scene, such as the implant or instrument position. For example, screws or plates may be virtually colored in real-time based on the bone location, thickness or quality they will encounter at their real-time position or based on simulated internal stress or fatigue resulting from intraoperative manipulation of the material. Another example may be to dynamically update the optimal screw trajectories based on the implant position. Yet another example is to update a simulation of the range of motion based on the implant position. This may be visualized by visualization module 110 on display device 104 in overlay or using a visualization that represents the difference or similarity between the diseased or native range of motion, the planned range of motion and the simulated range of motion based on the real-time implant position.

The user may look at the augmented environment 220 via display device 104 such as traditional computer displays, via mobile or portable devices such as smartphones or tablets, via projection-based devices or via head-mounted display systems. In some instances, the display will visualize the augmented environment 220 as a combination of virtual elements 208 and a camera image of the actual scene 230. In other instances, the display will only visualize the virtual elements of virtual scene 212 and project them in the field-of-view of the user as to overlay them on his own perception of the actual scene 230 (see-through display).

In certain embodiments, the visualization of a virtual scene 212 may be modified to improve visual access to the individual virtual objects in the AR system, for example, by exploding the view of a virtual model to its individual components. For example, a CMF surgical plan may include a (virtual) anatomical model of several bones as well as some implants. It may be difficult to inspect one of the objects (e.g., bone fragments) in the plan view. The user may explode the view whereby the individual components of the plan are, for example, radially displaced by a certain amount so that they can more easily be selected by the user.

Certain embodiments comprise methods of using visualization module 110 of the AR system before or during a surgical procedure as described herein.

Certain embodiments comprise methods wherein the visualization module 110 is used during a craniomaxillofacial surgical procedure as described herein.

Certain embodiments comprise methods wherein the visualization module 110 is used in combination with one or more modules of the AR system as described herein.

Certain embodiments comprise methods wherein the visualization module 110 is accessed at the virtual workbench as described herein.

Certain embodiments comprising methods of using the visualization module 110 for visualizing one or more anatomical landmarks (maxilla or parts of maxilla, mandible or parts of mandible, tooth, nerve, etc.) for an orthognathic surgery are described herein.

Certain embodiments comprising methods of using the visualization module 110 for visualizing one or more medical devices and their locations, positions and/or orientations (implants, screws, surgical guides, etc.) for an orthognathic surgery are described herein.

Certain embodiments comprising methods of using the visualization module 110 for visualizing one or more medical instruments and their trajectories, angulations or depths (drills, drill bits, saws, saw blades, bending pliers, etc.) for an orthognathic surgery are described herein.

Certain embodiments comprising methods of using the visualization module 110 for visualizing one or more anatomical landmarks (maxilla or parts of maxilla, mandible or parts of mandible, tooth, nerve, etc.) for a trauma surgery are described herein.

Certain embodiments comprising methods of using the visualization module 110 for visualizing one or more medical devices and their positions, locations and/or orientations (implants, screws, surgical guides, etc.) for a trauma surgery are described herein.

Certain embodiments comprising methods of using the visualization module 110 for visualizing one or more medical instruments and their trajectories, angulations or depths (drills, drill bits, saws, saw blades, bending pliers, etc.) for a trauma surgery are described herein.

Certain embodiments comprising methods of using the visualization module 110 for visualizing one or more anatomical landmarks (maxilla or parts of maxilla, mandible or parts of mandible, tooth, nerve, etc.) for a mandible reconstruction surgery are described herein.

Certain embodiments comprising methods of using the visualization module 110 for visualizing one or more medical devices and their positions, location and/or orientations (implants, screws, surgical guides, etc.) for a mandible reconstruction surgery are described herein.

Certain embodiments comprising methods of using the visualization module 110 for visualizing one or more medical instruments and their trajectories, angulations or depths (drills, drill bits, saws, saw blades, bending pliers, etc.) for a mandible reconstruction surgery are described herein.

Certain embodiments comprising methods of using the visualization module 110 for visualizing one or more anatomical landmarks (maxilla or parts of maxilla, mandible or parts of mandible, tooth, nerve, etc.) for a maxilla reconstruction surgery are described herein.

Certain embodiments comprising methods of using the visualization module 110 for visualizing one or more medical devices and their position and/or location (implants, screws, surgical guides, etc.) for a maxilla reconstruction surgery are described herein.

Certain embodiments comprising methods of using the visualization module 110 for visualizing one or more medical instruments and their trajectories, angulations or depths (drills, drill bits, saws, saw blades, bending pliers, etc.) for a maxilla reconstruction surgery are described herein.

Certain embodiments comprising methods using the visualization module 110 for visualizing one or more anatomical landmarks (orbit or parts of orbit, maxilla or parts of maxilla, mandible or parts of mandible, tooth, nerve, etc.) for an orbital-floor reconstruction surgery are described herein.

Certain embodiments comprising methods of using the visualization module 110 for visualizing one or more medical devices and their positions, locations and/or orientations (implants, screws, surgical guides, etc.) for an orbital-floor reconstruction surgery are described herein.

Certain embodiments comprising methods of using the visualization module 110 for visualizing one or more medical instruments and their trajectories, angulations or depths (drills, drill bits, saws, saw blades, bending pliers, etc.) for an orbital-floor reconstruction surgery are described herein.

Certain embodiments comprising methods of using the visualization module 110 for visualizing one or more anatomical landmarks (orbit or parts of orbit, maxilla or parts of maxilla, mandible or parts of mandible, tooth, nerve, etc.) for a tumor-resection and reconstruction surgery of one or more anatomical parts of the CMF region are described herein.

Certain embodiments comprising methods of using the visualization module 110 for visualizing one or more medical devices and their position, locations and/or orientations (implants, screws, surgical guides, etc.) for a tumor-resection and reconstruction surgery of one or more anatomical parts of the CMF region are described herein.

Certain embodiments comprising methods of using the visualization module 110 for visualizing one or more medical instruments and their trajectories, angulations or depths (drills, drill bits, saws, saw blades, bending pliers, etc.) for a tumor-resection and reconstruction surgery of one or more anatomical parts of the CMF region are described herein.

Certain embodiments comprising methods of using the visualization module 110 for visualizing one or more anatomical landmarks (orbit or parts of orbit, maxilla or parts of maxilla, mandible or parts of mandible, tooth, nerve, etc.) for a trauma surgery of one or more anatomical parts of the CMF region are described herein.

Certain embodiments comprising methods of using the visualization module 110 for visualizing one or more medical devices and their positions, locations and/or orientations (implants, screws, surgical guides, etc.) for a trauma surgery of one or more anatomical parts of the CMF region are described herein.

Certain embodiments comprising methods of using the visualization module 110 for visualizing one or more medical instruments and their trajectories, angulations or depths (drills, drill bits, saws, saw blades, bending pliers, etc.) for a trauma surgery of one or more anatomical parts of the CMF region are described herein.

Certain embodiments comprising methods of using the visualization module 110 for visualizing one or more anatomical landmarks (cranial vault, cranium, maxilla or parts of maxilla, mandible or parts of mandible, tooth, nerve, etc.) for a craniosynostosis surgery are described herein.

Certain embodiments comprising methods of using the visualization module 110 for visualizing one or more medical devices and their positions, locations and/or orientations (implants, screws, surgical guides, etc.) for a craniosynostosis surgery are described herein.

Certain embodiments comprising methods of using the visualization module 110 for visualizing one or more medical instruments and their trajectories, angulations or depths (drills, drill bits, saws, saw blades, bending pliers, etc.) for a craniosynostosis surgery are described herein.

### Calibration Module 112

In those embodiments where display device 104 comprises a see-through display, display calibration may be required (along with registration) to ensure that the display system understands how to align the viewing position of the user in relation to the display and create an experience where virtual information can spatially be overlaid correctly on the actual scene. Calibration may only be required in see-through displays and refers to the determination of the user's viewing perspective (eyes) relative to the see-through display. It determines where the display should render the image for the user to perceive the virtual elements at the correct location in space. Often eye tracking is used to determine a user's eye position in relation to the display.

Computing environment 100 may therefore include a calibration module 112 configured to perform such display calibration as part of the augmented-reality system. The calibration is user dependent and may be repeated if the display 104 is repositioned with respect to the user. Display calibration may also allow to correct for optical artifacts caused by glasses or surgical masks which may sit between user, the display and camera system and the environment. It may be performed by asking a user to perform any task where he aligns a physical element (e.g., a tracked marker, a body-part, an object, etc.) to one or multiple virtual elements displayed on the see-through display. To improve performance or user experience, display calibration may be performed interactively, whereby the display calibration is iteratively updated as the user is performing the task. The calibration module 112 may provide additional guidance to the user for performing display calibration, e.g., by using optical tracking to provide feedback on the distance from the user at which the task needs to be performed. Alternatively, display calibration can be performed using eye tracking or gaze tracking or using external camera systems that can spatially relate the positions of the display and the eyes.

Display calibration may be stored based on individual user profiles, so that for recurring sessions, the calibration effort can be eliminated or vastly reduced.

Multiple camera systems of I/O module 122 can be used and can be calibrated together or can be used to calibrate a single display when their relative position is known.

Certain embodiments comprise methods of using calibration module 112 of the AR system before or during a surgical procedure as described herein.

Certain embodiments comprise methods wherein the calibration module 112 is used during a craniomaxillofacial surgical procedure as described herein.

Certain embodiments comprise methods wherein the calibration module 112 is used in combination with one or more modules of the AR system as described herein.

Certain embodiments comprise methods of accessing the calibration 112 module at the virtual workbench as described herein.

Certain embodiments comprising methods of using the calibration module 112 during an orthognathic surgery are described herein.

Certain embodiments comprising methods of using the calibration module 112 during a trauma surgery are described herein.

Certain embodiments comprising methods of using the calibration module 112 during a mandible reconstruction surgery are described herein.

Certain embodiments comprising methods of using the calibration module 112 during a maxilla reconstruction surgery are described herein.

Certain embodiments comprising methods of using the calibration module 112 during an orbital-floor reconstruction surgery are described herein.

Certain embodiments comprising methods of using the calibration module 112 during a tumor-resection and reconstruction surgery of one or more anatomical parts of the CMF region are described herein.

Certain embodiments comprising methods of using the calibration module 112 during a trauma surgery of one or more anatomical parts of the CMF region are described herein.

Certain embodiments comprising methods of using the calibration module 112 during a craniosynostosis surgery, are described herein.

### Registration Module 114

As described above, the AR system needs to align the virtual environment/scene 212 and the actual scene 230 to create the ‘augmented environment/scene’ 220. This involves bringing the virtual and the actual scenes in a common coordinate system. This can be achieved through optical or other alignment systems, whereby the actual scene 230 is spatially referenced to the virtual environment 212. For example, in certain embodiments, computing environment 100 includes a registration module 114 to perform such alignment as described herein. Registration module 114 may be configured to register 218 a virtual scene 212 including virtual/augmentation elements 208 and/or statistics 206 for display, to actual scene 230, to generate augmented scene 220 which is then visualized 222 and output to display device 104.

In certain embodiments, the computing environment 100 comprises a registration module 114 to perform registration on a plurality of objects such as anatomical models, medical devices, medical instruments and parts of the patient. Registration may refer to the correct alignment of the virtual world with the real world. For example, registering a particular object may refer to aligning the real-world object correctly within the virtual world, such as into a world coordinate system, such that virtual objects in the virtual world appear in the correct location with respect to real-world objects in the real world in AR scenarios where visualization of the virtual world and real world are inter-mixed. Optical systems may use physical markers to perform registration 218 and tracking by registration module 114. These markers may be positioned in a fixed location in the actual scene 230, or be attached to any of the independently moving objects in the actual scene 230, such as one or more of the patient, any individually moving anatomical part of the patient (e.g., bones, soft-tissue, face, etc.), objects such as instruments (e.g., drills, saws, bone pins, etc.), surgical guides, anatomical 3-D patient-specific models, medical devices (e.g., implants or implant components), objects in the OR (e.g., surgical table, medical equipment, etc.), and/or the like. The markers may be organized in pre-defined 3-D configurations to facilitate optical recognition. Any physical 3-D object with a known geometry may directly serve as a physical marker. The marker may be a (e.g., 3-D-printed) surgical guide, which is (partially) patient specific and may be configurable intra-operatively to a certain degree, or an anatomical 3-D patient-specific model used for registration. Markers may also be included in wearable devices such as eyewear, elastic bands, dental splints, etc.

In some embodiments, the motion of a particular object, such as an instrument, pointer, a (part of) an imaging system, an implant, an implant component, the patient, an anatomical part of the patient, the user, an anatomical part of the user, the surgical staff, or an anatomical part of someone in the surgical staff, may be tracked through the actual scene, or the location and orientation of such an object may be determined to bring it into the world coordinate system or to attach the world coordinate system to such an object. A common way of doing this is to optically track the movement of one or multiple clearly identifiable markers rigidly attached to the object with one or more cameras or sensors either at fixed positions in the actual scene or incorporated into the display unit.

In certain embodiments, the AR system may be used to provide expert-based navigation using visual referencing between a virtual element and its physical counterpart in the real world. In certain embodiments, a user may use a physical registration object to determine a reference coordinate system to which the virtual elements and their visualizations are attached. The user will be presented with a real-time overlay of virtual objects in the reference coordinate system. In certain aspects, the physical registration object is an object of known (technical) geometry, such as a medical device (e.g., an off-the-shelf implant, a patient-specific implant, a patient-specific guide) or a surgical instrument (e.g., a power tool, a drill, a drill bit, a saw, a saw blade, a pair of bending pliers, a reamer, etc.). Further, in certain aspects, an object of known geometry may be a bone fragment or other anatomy separated from an individual such that, for example, its geometry can be fully scanned and determined to be used as a physical registration object. Such a physical registration object having a known geometry, unlike conventional patient anatomy for which the geometry is unknown precisely, may provide benefits over conventional AR systems, as further discussed herein.

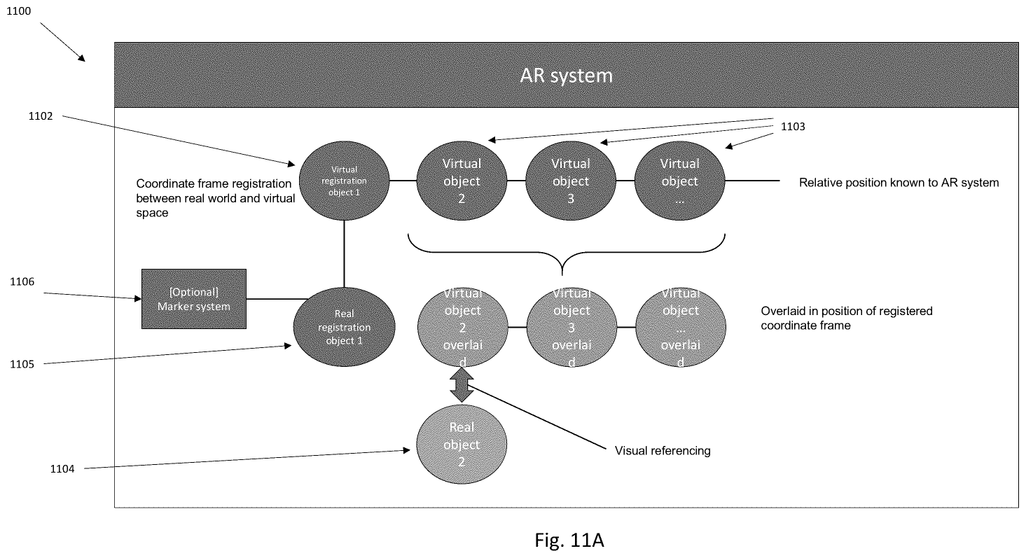

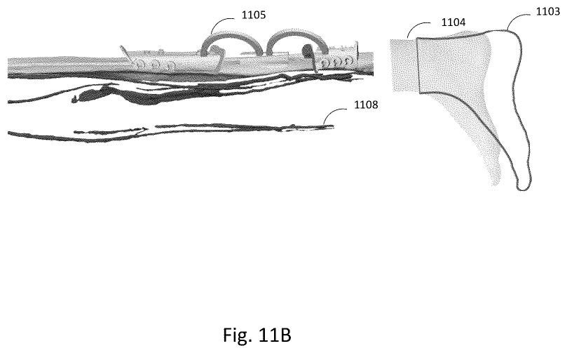

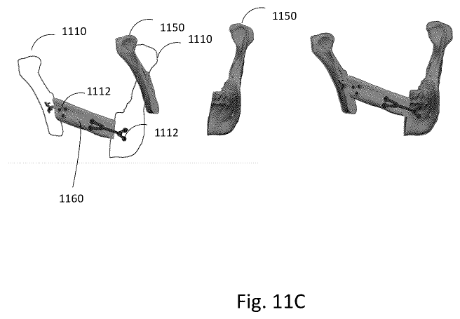

With reference to FIG. 11A, the registration between the virtual registration object 1102 and its corresponding real, physical registration object 1105 may be achieved through direct object tracking of the real registration object 1105 or, optionally, by attaching a tracker or marker system 1106 to the real object 1105. However, it should be noted that certain aspects are specifically directed to direct object tracking of a real registration object 1105 having a known geometry.

Such visual navigation would allow an expert user to compare the positions of anatomical structures relative to the position of the registration object 1105 in the real scene to how they were planned in the virtual scene. This can help with navigation, without requiring the AR system to take over the navigation entirely or to perform technically more challenging registration tasks.

In certain aspects, a workflow may start with virtual planning whereby one or more virtual 3-D anatomical models 1103 (which may include or comprise derived features such as landmarks) are generated from a medical image. Next, a surgical plan may be determined which describes the desired end result and the relative position of one or more physical objects (physical registration objects 1105) used during surgery so as to achieve this desired end result (such as implants, guides, markers, surgical instruments, such as drills, saws, reamers, bending pliers, etc.) with respect to the real counterparts 1104 of the virtual anatomical parts 1103. Those physical registration objects 1105 may be digitized as virtual registration objects 1102 in the AR system, for example because their geometry is known before manufacturing (such as in the case of additive manufacturing of patient-specific guides or patient-specific implants) or because their geometry was digitized after manufacturing (such as in the case of optical scanning of standard implants or surgical instruments).

Next, the user may load the surgical plan, including virtual anatomical models 1103 and virtual registration objects 1102, in the AR system. Next, the user may introduce the real registration object 1105 into the field of view of the sensors of the AR system. Next, the AR system may use any known method for registering or tracking a known object using the sensors (e.g., using pose estimation algorithms based on a video stream from a camera), and obtaining a coordinate frame transformation matrix. Next, the AR system may adapt the coordinate system where it visualizes the virtual objects 1103 (e.g., the anatomical models) based on the coordinate frame transformation matrix. Next, the AR system may visualize one or more of the virtual objects 1103 in the coordinate frame obtained using the registration object. Next, the user may rely upon a visual alignment of the visualization of virtual objects 1103 and their real counterparts 1104 to move real registration object 1105 into its planned position. The AR system may visualize the virtual registration object 1102 as an overlay on the real registration object 1105 for quality assurance reasons.

Such visual navigation allows an expert user to achieve the planned relative position of the real registration object 1105 with respect to the real object 1104 by moving the real registration object 1105 until the visualization of one or more virtual objects 1103 visually aligns with their real counterparts 1104. As described above, the use of a real registration object 1105 avoids using the patient for registration. Tracking the position of an object of a known (technical) geometry, such as a medical device (e.g., an off-the-shelf implant, a patient-specific implant, a patient-specific guide) or a surgical instrument (e.g., a power tool, a drill, a drill bit, a saw, a saw blade, a pair of bending pliers, a reamer, etc.) is technically much less challenging than directly tracking (part of) the patient anatomy, and therefore more robust. A way of making patient tracking more robust, known from navigation systems, is implanting a tracking marker into the patient. However, this is an additional invasive step, and requires careful determination of the relative position of the marker and the anatomy. The use of a registration object 1105 avoids these disadvantages.

In some embodiments, multiple registration objects 1105 may be used to determine multiple coordinate transformations for the virtual objects 1103. This would be helpful for example in articulating objects such as mandible and maxilla.

In an example embodiment, a physical registration object 1105 may be a cutting guide for a fibula as shown in FIG. 11B. Virtual objects 1103 that share the reference coordinate frame in the virtual space with the registration object may be anatomical landmarks, parts of the anatomy (e.g., fibula bone, soft tissue of the ankle), etc. In the example shown, the virtual objects 1103 may include a virtual outline of a foot of the patient. First, a user may perform surgical planning to determine the desired relative position of the cutting guide 1105 with respect to the other objects, e.g., a part of the patient's anatomy. For example, the plan may indicate a location in space (position, orientation, etc.) in which a virtual model 1102 of the cutting guide 1105 should be placed relative to one or more virtual objects 1103. In this case, the plan may indicate a desired location of cutting guide 1105 relative to the patient's foot 1104. Next, the real cutting guide 1105 and its virtual counterpart 1102 are registered, for example through object tracking. Then, when the user views the cutting guide 1105 in the real world using an AR display device 104 as discussed, the AR display device 104 may display to the user the one or more virtual objects 1103 indicated in the surgical plan in a relative location with respect to the real cutting guide 1105, the relative location being the defined, desired relative location in the surgical plan. Accordingly, when the user moves the cutting guide 1105, the displayed one or more virtual objects 1103 correspondingly move in the display device, such that the relative position between the cutting guide 1105 and the visualization of the one or more virtual objects 1103 is maintained. The user may thus see the real-time position of the one or more virtual objects 1103, such as the outline of the patient's foot or other anatomical landmarks, in the same coordinate frame as the cutting guide 1105. The user may use this visualization to determine the correct position of the cutting guide 1105 on the fibula through a visual reference. For example, the user may move the cutting guide 1105 until the one or more virtual objects 1103 align with real objects 1104 in the user's view, such as aligning the patient's actual foot 1104 with the displayed outline of the patient's foot as a virtual object 1103. The cutting guide 1105 may then be considered correctly positioned, such as on the fibula, based on its relative position with respect to the patient's foot. Once the guide 1105 is correctly positioned on the fibula, the user may instruct the AR system to visualize additional anatomical elements such as soft-tissue outlines 1108 or vasculature to further support his surgical decisions, or to modify the surgical plan accordingly.

In another embodiment, this system may be used to perform the validation of a surgical guide fit. For example, in a mandible osteotomy, a mandibular cutting guide may be used as a registration object 1105. A user may use anatomical landmarks 1103 such as teeth, soft-tissue outlines and/or bony landmarks to validate the correct position of the surgical guide in relation to its pre-planned position. If the surgical guide is misplaced, the user may visually see a mismatch between the real anatomical landmarks 1104 and their virtual counterparts 1103. Using this system, a surgical guide may be made more compact as the use of the AR system may obviate the need to have a large patient-specific anatomy-matching support surface on the guide, or even to have a unique patient-specific surgical guide.

In another embodiment, this system may be used to position implants such as a temporo-mandibular-joint (TMJ) implant or an orbital-floor implant. For example, the implants may be rigidly attached to a holding instrument which may also be the registration object 1105 as the small incisions may require a surrogate object for tracking. Again, anatomical landmarks 1104 such as the dental arch, nose, face, and their virtual counterparts 1103 may be used as a visual reference for the surgeon.

In another embodiment, this system may be used in craniosynostosis or craniotomy surgery. Here, the registration object 1105 may be one or more cutting guides. The surgeon may be presented with an overlay of the desired surgical outcome after repositioning bone fragments.

In another embodiment, this system may be used in craniosynostosis or craniotomy surgery to provide support during bone fragment shaping. The planned bone fragments may be used as registration objects and the desired deformation or adaptations of the bone fragments may be shown in overlay.

In another embodiment, this system may be used to visualize information related to surgical tools that may be used during a surgical procedure. For example, a registration object may be a surgical guide or an implant, the virtual objects that are overlaid could be surgical screws (or an annotation of their desired size or length) or diameters of predrilling holes.

In another embodiment, the system may be used during an osteotomy. A surgical guide attached to the patient's anatomy or a dental splint attached to the patient's dentition may be used as a registration object. The user may be presented with safety zones or margins where an osteotomy may be executed. The user may also be presented with desired osteotomy planes for visual referencing and alignment with a sawblade.

In another embodiment, the system may be used to add virtual information to a medical instrument. Here, a cutting or drilling guide may be used as a registration object. The user may be presented with virtual drilling trajectories or cutting planes aligned with drilling holes or cutting flanges or slots. Based on this information, the user may be able to better estimate the correct drilling angle or osteotomy orientation, avoiding a possible misalignment due to the limited restrictions to the degrees of freedom imposed by the drill holes.

In another embodiment, the system may be used to provide depth guidance for drilling or sawing. The registration object may be a cutting or drilling guide. The user may position the guide on the anatomy. The user may then position a drill or sawblade in the foreseen drilling holes or cutting slots. The user may see the depth of the drilling hole or cut projected on the real drill or saw blade (the depth is inversed and overlaid on the drilling or cutting trajectory). The user may then mark the physical drill or saw blade with a visual reference (e.g., drawing with a surgical marker or attaching a clip to the drill or saw blade at the desired depth). The user may then drill the hole or make the cut to the desired depth. The system may take the thickness of the drilling or cutting guide into account and visualize to the user the distance to the edge of the guide rather than to the edge of the bone. Alternatively, upon first insertion of the real drill bit or saw blade into the guide, the system may register its proximal end (e.g., where the drill bit enters the power tool, or where the saw blade attaches to the saw) as a baseline. Then, based on this baseline and the desired depth, the system may project onto the surgical tool a visual reference, such as the baseline moved over the desired drilling or cutting depth towards the patient anatomy. The user may then operate the drill or saw until the proximal end of the drill bit or saw blade matches the visual reference.

In another embodiment, the system may be used to support navigation of tasks such as sawing, drilling, reaming or taking biopsies. The registration object may be a drill, a saw, a reamer or a biopsy needle. The trajectory of the drill, saw, reamer or biopsy needle may be determined during surgical planning. The user may see the anatomy as it should be during one or multiple stages of executing the navigated task, attached to the registration object. For example, initially, the user may see the anatomy in overlay when the sawblade, drill bit, reamer or biopsy needle enters the tissue, so as to allow the user to visually find the planned trajectory. Afterwards, while entering the sawblade, drill bit, reamer or biopsy needle into the anatomy, he may see the anatomy in overlay in one or more consecutive intermediate desired positions and/or the final desired position of the biopsy.

In another embodiment, the system may be used to attach/fix implants to bone fragments as shown in FIG. 11C. The registration object may be an implant 1112. The virtual elements shown may be 3-D models of one or more bone fragments 1110 (here shown as outlines, although other visualizations are also possible). The user may position the implant 1112 on the actual bone 1150 by using the visual reference of the virtual bone fragments 1110 to determine the correct position as shown in FIG. 11C. The figure illustrates the exemplary fixation of a bone graft 1160 to two bone fragments 1150 of a mandible in a mandible reconstruction surgery. The described method may be used first for correctly attaching implant 1112 to bone graft 1160 and subsequently for correctly attaching the construct of implants 1112 and bone graft 1160 to bone portions 1150.

Alternatively or additionally, markers may be attached to multiple structures or parts thereof (e.g., cranium, eyelids, teeth, nose, neck, maxilla, mandible or cars.) and indirectly related to the anatomy to be registered by using computational models.

A marking device may also be used (e.g., a tracked pen or stylus) via I/O module 122 to register the anatomy of individual body parts, such as bones, in the augmented environment. Here, the user will use the marking device in the coordinate frame of a reference marker (e.g., attached to the anatomy or environment) to determine the location of at least 3 points on the anatomy of the patient with a known counterpart in the virtual 3-D model of the anatomy. This can be done discretely or continuously using for example voice commands to trigger the storage of one or more points in the AR system. The coordinates of these points in the coordinate system of the reference marker can then be used in an algorithm (such as iterative closest point) to determine the registration matrix between the reference coordinate frame of the real world and the coordinate frame of the virtual anatomy. This may include the dental surface. The dental surface may also be used as a reference point. The combination of multiple markers, in combination with vision data (e.g., from cameras of I/O module 122), may also be used to capture the anatomy of interest's position.

Although technically more challenging, as described above, optical systems may also operate without markers by directly using the anatomy visible inside the surgical window or adjacent structures (such as soft-tissue or skin) around the surgical window for registration and tracking by registration module 114.

Information such as position and location of an anatomical part may be used by the registration module 114 and by the virtual-3-D-model-creation module 106 to register one or more anatomical parts with their virtual 3-D anatomical models created by the virtual-3-D-model-creation module 106. Registration module 114 may register (e.g., parts of) the virtual 3-D model created by virtual-3-D-model-creation module 106 to data acquired from a camera system or surface-scanning system by I/O module 122 by using any shape-recognition techniques known in the art. The 3-D model may be a patient-specific model based on imaging, may be an instance of a parametric model, e.g., derived from a population (such as a statistical shape model), a generic statistical shape model or may be obtained intra-operatively. Such a vision-based system may also directly track the instruments and their position in space, without the need for explicit marker systems. The aforementioned vision system (e.g., optical scanning of the face, intra-operative 3-D imaging) may also be used to determine the relative position of any fixed bone markers (e.g., attached to the cranium) relative to the virtual plan in an initial or intermediate stage, after which the fixed bone markers may be used to track the patient during surgery. Annotations may be created (e.g., drawn or by means of stickers optimized for respective vision system) on the patients' skin (e.g., marking points) to aid the system in the registration. As mentioned above, direct registration of a virtual 3-D model of patient's anatomy onto the real patient anatomy may be challenging, depending on the nature of the anatomical part that is operated on, the size of the surgical window, the nature of the anatomy surrounding the surgical window, the presence of tissue obscuring the view, etc. However, such direct registration may be made easier and more robust by referencing discernible anatomical landmarks, such as surface features on the surface of a bone, tooth, organ or tissue (e.g., points, lines, or areas exhibiting a small curvature radius, or a curvature radius differing from surrounding surfaces, such as bumps, indentations, ridges, grooves, apertures, notches, cusps, fissures, foramina, fossae, foveae, tubercles, tuberosities, trochanters, processes, condyles, epicondyles, etc.), edges of, borders between and/or spaces between bones, teeth, organs or tissues (e.g., sutures, fontanelles, borders between teeth, border of a cartilage region, gumlines, borders between teeth and exposed jawbones, borders of ligament attachment areas, borders of tendon attachment areas, borders of menisci, etc.), visible damage to tissue (e.g., scars, fractures, osteophytes, cartilage damage, tooth cavities, etc.) or points, lines, or areas exhibiting a color different from the surrounding surfaces (e.g., skin discolorations, freckles, beauty marks, tooth discolorations, bone discolorations, irises, tattoos, stickers, marking points, etc.). Robustness can be improved even more by explicitly identifying such features in the virtual 3-D model of the patient's anatomy as separate virtual entities, such as points, lines, polylines, curves, or surfaces. Some such features may not be available in the original medical images from which the virtual 3-D model is derived, e.g., because the boundary between adjacent tissues might not be clearly visible in a particular image modality, because the resolution of a particular image modality is insufficient, or because tissue color is not visible in a particular image modality. To overcome this, a multi-modality virtual 3-D model may be constructed. For example, a CT scan of a part of the patient's craniomaxillofacial area may be combined with a higher-resolution intraoral scan of the patient's dentition or a higher-resolution optical scan of a plaster cast of the patient's dentition, so as to generate a single virtual 3-D model that combines the overall shape of the bony anatomy and the finer geometric detail of the dentition. As another example, data from one or more visible-light cameras, such as cameras of the I/O module 122, may be mapped pre-operatively or intra-operatively onto a virtual 3-D model of the patient's anatomy to add color information.

The AR system may use one or more purposely created devices of which the unique fit to the anatomy is known in the virtual space. Such devices can include (dental) splints, glasses, earplugs, facebows, stereotactic frames, etc. The aforementioned devices may also be generic (not patient specific) or semi-patient-specific devices but through their design have a known position on the patient, e.g., the touchpoints on the teeth can be uniquely predicted based on a virtual fit.

An incremental registration (or layering) mechanism may also be used by registration module 114 whereby initially the scene is registered to the anatomy using for example a cutting guide. Accordingly, virtual objects are displayed in the scene based on their relative position as planned with respect to the cutting guide. In a second stage the scene is for example registered to an implant component (e.g., a marker on the implant or the implant itself as the marker). Accordingly, virtual objects are displayed in the scene based on their relative position as planned with respect to the implant component. In particular, a scene being “registered” to a physical registration object may mean that virtual objects are then displayed in locations in the scene defined relative to the registration object. Such an incremental registration mechanism is useful as it provides the user with the ability to use existing items, such as instrumentation that is part of the surgical workflow, and use these for registration, even if the anatomy is changing during surgery. For example, the surgeon may position a surgical guide to perform predrilling of holes for implant fixation elements, such as screws, and an osteotomy on the mandible. The surgical guide may have been configured, e.g., pre-operatively, to comprise a support surface that matches the shape of the patient's anatomy and as such may physically perform the anatomical registration which provides the surgeon with initial guidance in the first phase of surgery, e.g., by allowing the AR system to use the surgical guide as a physical registration object and subsequently visualizing the planned orientation of a drill trajectory or sawblade. After the cut and predrilling have been performed, the surgeon can attach an implant (component), such as an osteosynthesis plate, to one of the bone fragments. At that point, the implant (component) can take over the function of the physical registration object for the second part of the surgery, e.g., to visualize additional steps to be performed.

In some embodiments, the motion of a particular object, such as an instrument, pointer, a (part of) an imaging system, an implant, an implant component, the patient, an anatomical part of the patient, the user, an anatomical part of the user, the surgical staff, or an anatomical part of someone in the surgical staff, may be tracked through the actual scene, or the location and orientation of such an object may be determined to bring it into the world coordinate system or to attach the world coordinate system to such an object. A common way of doing this, is to optically track the movement of one or multiple clearly identifiable markers rigidly attached to the object with one or more cameras or sensors either at fixed positions in the actual scene or incorporated into the display unit 104.

Alternative ways may include creating a digital model of the object itself and using shape recognition techniques to optically track the movement of the object itself (e.g., object tracking) or using stochastic methods (e.g., for hand tracking) to track such objects. Yet other methods may use alternative sensor systems such as medical imaging, radio-wave based positioning (RFID, UWB), image-based tracking, etc. They may be referenced to the augmented environment through visual systems or through otherwise calibrated electronic systems.

In certain embodiments, the AR system is used for registering and tracking devices such as medical instruments. As described earlier, registration of medical instruments may be done using one or more of optical markers, implantable/attachable markers, patient-specific markers, markers of different shapes and sizes (e.g., circle, square, rectangle, triangle, black square, blue circle, red triangle, etc.). Registration of medical instruments may be done using any known registration technique such as optical tracking, spatial mapping, light sources, intra-op imaging, etc. The information may be stored in the database for easy retrieval during other surgeries. For example, using the medical inventory, a user may link a digital tag to a medical instrument. The digital tag may be any type of information that the user considers worth storing, such as whether the instrument was used during surgery, at what point in time it was used, at what position, e.g., relative to the patient, it was used, etc.

Previous implant components or dedicated tracking components implanted in an additional surgery performed upfront may be detected automatically in surgery. The implant components may then be used as registration markers as they may be visible in pre-surgical imaging data and their spatial relationship with patient anatomy may therefore be derived from said imaging data.

Registration and tracking allow the user to perform various measurements 232, as described herein.

As the augmented-reality system/environment 100 allows to estimate the pose and location of anatomy and instruments, it may be used to make measurements 232 intra-operatively or pre-operatively. In certain instances, the true-to-scale dimensions of anatomy or instruments are known preoperatively, e.g., based on medical imaging data or based on a CAD model of an implant. After registration, this provides a coordinate frame which allows the user to perform measurements which can be transformed into standard measurement units such as mm or inches. These measurements may influence the planning procedure 210 or allow to display specific information.

Examples of measurements that could be performed involve the parameters for shaping (e.g., bending) a standard implant, such as a plate, or the parameters of a cut based on a sawblade position. Such parameters could include one or more of indication of cutting, angulation of bending, placement of screw holes, amount of bone to be resected, etc.

Another example of a measurement that could be performed is the determination of the natural head position, e.g., by means of the Frankfort horizontal plane (i.e., the plane through left and right portion and left orbitale) or the sella-nasion plane (i.e., the plane formed by projecting a plane from the sella-nasion line).

Another example of a measurement that could be performed is the determination of parameters that define soft-tissue characteristics such as tissue elasticity to improve upon a preoperatively determined soft-tissue simulation or execute a soft-tissue simulation intraoperatively.

Another example of a measurement may be the determination of the parameters for plate shaping (e.g., bending) based on, e.g., an analysis of the plate available, and/or of suitable locations for drilling holes (e.g., drill location, depth, angulation, etc.) based on an analysis of the drill position.

Also, anatomical landmarks in the virtual or real world can be manually annotated using a tracked object (including hands or parts thereof), device or pointer or by visually detecting the landmarks through the camera by I/O module 122. From these anatomical landmarks, measurements can be derived.

Automatic detection of the exposed bone or soft-tissue surface can allow creating a 3-D representation of this anatomy. This may be used for example to detect if the bone shape still corresponds to the original image data (e.g., to check that a recent scan was used) or to identify if soft-tissue changes have occurred that may influence the outcome (e.g., oncology or trauma).

Another exemplary measurement may be the determination of the range of motion of the jaw, pre-operatively as well as intra-operatively. By performing passive or active motion and tracking the anatomical components, an analysis of this motion can be made, e.g., to determine the alignment between the maxilla and the mandible and to determine the movement of the TMJ joint. The measurement may be performed on the actual jaw of a patient or may be visualized on a virtual anatomical model.

During range-of-motion assessment, the surgeon is moving the jaw. Using intra-operative measurement tools (such as sensors), more objective assessment of the range-of-motion may be achieved. Alternatively, using AR the displacement of the jaw may be controlled, for example by visualizing target angles (such as represented by planes, lines, cones, etc.) in overlay. Optionally, this data may also be stored in or retrieved from the scanning-device and image-storage module 105.

The registration may be achieved by registration module 114 through landmark-driven methods (e.g., identifying corresponding landmarks in the camera data and on the virtual model, aligning any set of landmarks obtained from the patient with their counterparts in the virtual model), painting-based methods (e.g., annotating parts of an anatomical surface and registering it to a surface of a virtual model), projection-based methods (e.g., by optimizing the registration through image comparison of the camera data with a rendered 3-D model), surface-scanning methods (e.g., by using a depth camera or time-of-flight image), through machine-learning techniques (e.g., by learning the appearance of a 3-D model in the camera through data-driven training and estimating the pose), or other methods.

For example, the user may be asked to use a pointer to outline discernible anatomical features, such as the edges of the teeth or nerves or tumor or the patient's dentition, e.g., on a digital model using a computing device, in a virtual scene while the I/O module 122 captures movement of the pointer, etc., whereby the pre-segmented outlines of those anatomical features based on pre-surgical CT, CBCT or optical scans can be used to perform the registration.

The user may be asked to annotate parts of the anatomy, e.g., by coloring the teeth or nerves or drawing a pattern on the bony anatomy, e.g., on a digital model using a computing device, in a virtual scene while the I/O module 122 captures movement of the pointer, etc., so as to allow optical recognition algorithms to operate fully autonomously. This type of annotation is particularly helpful for ill-defined anatomical parts such as tumors wherein the boundaries for resection are annotated by drawing.

The user may be asked to annotate parts of the anatomy, e.g., by highlighting a part of the bony anatomy, e.g., on a physical anatomical 3-D model corresponding to the parts of the anatomy, such as a physical patient-specific model, using one or more markers while the I/O module 122 captures movement of the pointer, etc., so as to allow registration.

The user may be asked to annotate parts of an implant, e.g., by highlighting parts of a plate (or other type of implant) which need to be bent, expanded, or cut, e.g., on a virtual 3-D model corresponding to the actual plate (or other type of implant), using one or more markers or a tracked pointer while the I/O module 122 captures movement of the pointer, etc., so as to allow registration.

User actions for registration 218 may involve the handling of a tracked pointer or object, the surgeon's hands or fingers. It may involve the user moving the pointer itself in the physical space or (actively or passively) moving the camera that is registering the pointer (e.g., by moving the user's head when wearing an OHMD).

Certain embodiments comprise methods of using registration module 114 of the AR system before or during a surgical procedure as described herein.

Certain embodiments comprise methods wherein the registration module 114 is used during a craniomaxillofacial surgical procedure as described herein.

Certain embodiments comprise methods wherein the registration module 114 is used in combination with one or more modules of the AR system as described herein.

Certain embodiments comprise methods of accessing the registration 114 module at the virtual workbench as described herein.

Certain embodiments comprising methods of using the registration module 114 for registering one or more anatomical landmarks (maxilla or parts of maxilla, mandible or parts of mandible, tooth, nerve, etc.) for an orthognathic surgery are described herein.

Certain embodiments comprising methods of using the registration module 114 for registering one or more medical devices and their position and/or location (implants, screws, surgical guides, etc.) for an orthognathic surgery are described herein.

Certain embodiments comprising methods of using the registration module 114 for registering one or more medical instruments and their trajectories, angulations or depths (drill bits, saw blades, bending pliers, etc.) for an orthognathic surgery are described herein.

Certain embodiments comprising methods of using the registration module 114 for registering one or more anatomical landmarks (maxilla or parts of maxilla, mandible or parts of mandible, tooth, nerve, etc.) for a trauma surgery are described herein.

Certain embodiments comprising methods of using the registration module 114 for registering one or more medical devices and their position, orientation and/or location (implants, screws, surgical guides, surgical instruments, etc.) for a trauma surgery are described herein.

Certain embodiments comprising methods of using the registration module 114 for registering one or more medical instruments and their trajectories, angulations or depths (drill bits, saw blades, bending pliers, etc.) for a trauma surgery are described herein.

Certain embodiments comprising methods of using the registration module 114 for registering one or more anatomical landmarks (maxilla or parts of maxilla, mandible or parts of mandible, tooth, nerve, etc.) for a mandible reconstruction surgery are described herein.

Certain embodiments comprising methods of using the registration module 114 for registering one or more medical devices and their position, orientation and/or location (implants, screws, surgical guides, etc.) for a mandible reconstruction surgery are described herein.

Certain embodiments comprising methods of using the registration module 114 for registering one or more medical instruments and their trajectories, angulations or depths (drill bits, saw blades, bending pliers, etc.) for a mandible reconstruction surgery are described herein.

Certain embodiments comprising methods of using the registration module 114 for registering one or more anatomical landmarks (maxilla or parts of maxilla, mandible or parts of mandible, tooth, nerve, etc.) for a maxilla reconstruction surgery are described herein.

Certain embodiments comprising methods of using the registration module 114 for registering one or more medical devices and their position, orientation and/or location (implants, screws, surgical guides, etc.) for a maxilla reconstruction surgery are described herein.

Certain embodiments comprising methods of using the registration module 114 for registering one or more medical instruments and their trajectories, angulations or depths (drill bits, saw blades, bending pliers, etc.) for a maxilla reconstruction surgery are described herein.

Certain embodiments comprising methods using the registration module 114 for registering one or more anatomical landmarks (orbit or parts of orbit, maxilla or parts of maxilla, mandible or parts of mandible, tooth, nerve, etc.) for an orbital-floor reconstruction surgery are described herein.

Certain embodiments comprising methods of using the registration module 114 for registering one or more medical devices and their position, orientation and/or location (implants, screws, surgical guides, etc.) for an orbital-floor reconstruction surgery are described herein.

Certain embodiments comprising methods of using the registration module 114 for registering one or more medical instruments and their trajectories, angulations or depths (drill bits, saw blades, bending pliers, etc.) for an orbital-floor reconstruction surgery are described herein.

Certain embodiments comprising methods of using the registration module 114 for registering one or more anatomical landmarks (orbit or parts of orbit, maxilla or parts of maxilla, mandible or parts of mandible, tooth, nerve, etc.) for a tumor-resection and reconstruction surgery of one or more anatomical parts of the CMF region are described herein.

Certain embodiments comprising methods of using the registration module 114 for registering one or more medical devices and their position, orientation and/or location (implants, screws, surgical guides, etc.) for a tumor-resection and reconstruction surgery of one or more anatomical parts of the CMF region are described herein.

Certain embodiments comprising methods of using the registration module 114 for registering one or more medical instruments and their trajectories, angulations or depths (drill bits, saw blades, bending pliers, etc.) for a tumor-resection and reconstruction surgery of one or more anatomical parts of the CMF region are described herein.

Certain embodiments comprising methods of using the registration module 114 for registering one or more anatomical landmarks (orbit or parts of orbit, maxilla or parts of maxilla, mandible or parts of mandible, tooth, nerve, etc.) for a trauma surgery of one or more anatomical parts of the CMF region are described herein.

Certain embodiments comprising methods of using the registration module 114 for registering one or more medical devices and their position and/or location (implants, screws, surgical guides, etc.) for a trauma surgery of one or more anatomical parts of the CMF region are described herein.

Certain embodiments comprising methods of using the registration module 114 for registering one or more medical instruments and their trajectories, angulations or depths (drill bits, saw blades, bending pliers, etc.) for a trauma surgery of one or more anatomical parts of the CMF region are described herein.

Certain embodiments comprising methods of using the registration module 114 for registering one or more anatomical landmarks (cranial vault, cranium, maxilla or parts of maxilla, mandible or parts of mandible, tooth, nerve, etc.) for a craniosynostosis surgery are described herein.

Certain embodiments comprising methods of using the registration module 114 for registering one or more medical devices and their position, orientation and/or location (implants, screws, surgical guides, etc.) for a craniosynostosis surgery are described herein.

Certain embodiments comprising methods of using the registration module 114 for registering one or more medical instruments and their trajectories, angulations or depths (drill bits, saw blades, bending pliers, etc.) for a craniosynostosis surgery are described herein.

### Guidance Module 116

In certain embodiments, the computing environment 100 comprises a guidance module 116 to provide guidance to the user during the surgery. Guidance module 116 may do so by determining one or more virtual elements to be visualized, e.g., by visualization module 110, as part of augmented scene 220 and displayed by display device 104. Guidance module 116 may determine the position relative to the physical scene 230 in which the virtual elements are displayed and the way in which they are displayed.

For example, the user may receive guidance 214, by guidance module 116, for intra-operative annotation of landmarks, surface features or any other geometric or otherwise discernible features that may subsequently be used by registration module 114 for registering a physical object to a virtual object, either via a physical object (e.g., a 3-D-printed anatomical model with indicated registration landmarks) or via a virtual object displayed (e.g., in a workbench, free-floating, etc.) in the augmented scene. This guidance may show which landmarks need to be marked or annotated and in which order, it may show which parts of the patient anatomy are reliable for registration during marking-either for computational reasons (e.g., not all points can be coplanar) or because specific parts of the anatomy are not well known in the virtual space (e.g., the virtual anatomical model is created based on sparse data such as X-ray imaging and a population model, and is thereby only reliable at locations where information is available in the X-ray).

This guidance may be aligned and updated with the user's steps and as he/she moves forward through the workflow (e.g., by highlighting consequential landmarks after the previous ones have been annotated successfully, providing step-by-step guidance during the plate bending process as described below, showing trajectories for surgical instruments, such as drills, saws or reamers, etc.). Using the AR system, the surgical phase may also be detected (e.g., semi automatically). For example, the AR system may be trained using neural networks or other technologies to recognize certain actions that a surgeon performs or items (e.g., surgical instruments or implants) that a surgeon uses based on the embedded camera in the AR system. Alternatively, the surgeon can use voice commands to indicate to the AR system that a certain surgical phase is reached. This may lead to specific workflow guidance through the surgical procedure, e.g., by showing surgical steps, demonstrating the next step, providing up-to-date instrumentation lists, visualizing relevant virtual elements, etc. For example, in certain aspects, computing environment 100 includes a guidance module 116 configured to provide virtual surgical guidance 214. This may for example include providing guidance 214 during placement of one or more bone fixation plates, but it may also include aiding in the other steps in the procedure such as shaping grafts for trauma surgeries or showing graft placement required during reconstruction surgeries or marking of a tumor required to be resected.

One or more camera systems may be a part of the augmented environment. Also, one or more display devices 104 may be part of the AR system. Guidance module 116 may determine different virtual elements to be displayed by different display devices 104, e.g., depending on the role of the user as member of the surgical staff. Each display device 104 may therefore display a different augmented scene. Additionally, the AR system enables the user to lock in their camera view for a limited period of time (determined by the user). This locked camera view, or the augmented scene that is based on the locked camera view may subsequently be displayed on other display devices 104. This allows the surgeon to take a break in case of long, complex surgeries or to relay information to staff without losing relevant information while gathering data in real time. For example, the surgeon may require a third opinion during the surgery; for this, he/she may use the tele-surgery option to dial in another surgeon. To be able to get an opinion from the other surgeon, the user first locks the scene in his view such that during the discussion with the other surgeon, the locked scene may be displayed to the other surgeon. The changes suggested via tele surgery are overlaid on the locked scene, updated in real time for the surgeon in the OR to consider. If the user (surgeon in the OR) then wishes for the surgical plan to be updated as per the suggestion, the surgical plan is updated or else the surgeon can go back to the initial plan (as it was when the scene was locked in), i.e., the initial data/plan is not lost and navigating is still easy.

It is also possible to hold or lock the scene (e.g., temporarily) even if the surgeon takes the glasses off or removes markers or in the case of occlusion. This is especially helpful while bending a standard plate wherein the surgeon may prefer to confirm the bent plate against a virtual anatomical model or the patient directly intra-op.

Certain embodiments comprise methods of using guidance module 116 of the AR system during a surgical procedure as described herein.

Certain embodiments comprise methods wherein the calibration module 116 is used during a craniomaxillofacial surgical procedure as described herein.

Certain embodiments comprise methods wherein the calibration module 116 is used in combination with one or more modules of the AR system as described herein.

Certain embodiments comprising methods of accessing the guidance module 116 at the virtual workbench are described herein.

Certain embodiments comprising methods of using the guidance module 116 for guidance during an orthognathic surgery are described herein.

Certain embodiments comprising methods of using the guidance module 116 for guidance during a trauma surgery are described herein.

Certain embodiments comprising methods of using the guidance module 116 for guidance during a mandible reconstruction surgery are described herein.

Certain embodiments comprising methods of using the guidance module 116 for guidance during a maxilla reconstruction surgery are described herein.

Certain embodiments comprising methods of using the guidance module 116 for guidance during an orbital-floor reconstruction surgery are described herein.

Certain embodiments comprising methods of using the guidance module 116 for guidance during a tumor-resection and reconstruction surgery of one or more anatomical parts of the CMF region are described herein.

Certain embodiments comprising methods of using the guidance module 116 for guidance during a trauma surgery of one or more anatomical parts of the CMF region are described herein.

Certain embodiments comprising methods of using the guidance module 116 for guidance during a craniosynostosis surgery are described herein.

### Control Module 124

The computing environment 100 may also include a control module 124 that is connected to operate one or more external systems or apparatuses.

In certain aspects, an external system includes one or more manufacturing devices, such as additive-manufacturing (AM) devices (e.g., 3-D printers). The manufacturing device is directly connected to the AR system via the network 101. Accordingly, a person skilled in the art will understand that an manufacturing device, such as an additive-manufacturing device, may be directly connected to a standalone computer that is in turn connected to network 101, connected to a computer via a network 101, and/or connected to a computer via another computer and the network 101.

An additive-manufacturing device may run on any standard additive-manufacturing operating software and may be operable by any skilled person capable of using the additive-manufacturing device such as a nurse, technician, a clinical engineer, medical professional, etc.

A digital representation of an object (e.g., an implant, a surgical guides, an anatomical model, a graft, a graft cage, a filler, a spacer, etc.) may be designed or generated in accordance with pre-op or post-op or intra-op plans, e.g. using the virtual workbench, or retrieved from the scanning-device and image-storage module 105. For example, two-dimensional (2-D) or 3-D data, e.g. data representing patient anatomy, may be used to design the 3-D representation of the object. Alternatively, the digital representation may be retrieved from the scanning-device and image-storage module 105. The shape information corresponding to the 3-D object may be sent to an additive-manufacturing device and the additive-manufacturing device commences a manufacturing process for generating the physical 3-D object in accordance with the received shape information. The additive-manufacturing device manufactures the 3-D object using suitable, e.g., biocompatible, materials, such as a polymer, or metal powder, and the physical 3-D object is generated. The additive-manufacturing device may use known technologies such as fused deposition modeling (FDM), selective laser sintering (SLS), selective laser melting (SLM) or stereolithography (SLA) with any suitable material such as polyamide, titanium, titanium alloy, stainless steel, polyether ether ketone (PEEK), etc. Alternatively, the shape information corresponding to the 3-D object may be sent to a different type of manufacturing device, such as a CNC machine, for example a milling device. The milling device may then mill the physical 3-D object out of a die of suitable material. For example, a dental crown may be milled out of a die of a ceramic material, such as a zirconium oxide die.

Other miscellaneous items that may be required during surgery such as surgical tags or registration or tracking aids may also be manufactured. Once manufactured, the 3-D object may be registered using the registration module 114 to be tracked in the AR environment. Access to an additive-manufacturing device or another type of manufacturing device in the sterile environment of the OR allows the user to manufacture (or print) parts or whole tools and/or devices which otherwise would have to be manufactured beforehand. This is especially useful in emergency situations when the surgeon has limited time to plan a surgery.

In certain embodiments, an external system integrated in the AR system may be a robotic arm. A user of the AR system may be able to control the robotic arm during a surgical procedure as described herein.

Certain embodiments comprising methods of using control module 124 of the AR system during a surgical procedure are described herein.

Certain embodiments comprise methods wherein the control module 124 is used during a craniomaxillofacial surgical procedure as described herein.

Certain embodiments comprise methods wherein the control module 124 is used in combination with one or more modules of the AR system as described herein.

Certain embodiments comprising methods of accessing the control module 124 at the virtual workbench are described herein.

In certain embodiments the control module 124 may be used for operating one or more external systems during an orthognathic surgery, as described herein.

In certain embodiments the control module 124 may be used for operating one or more external systems during a trauma surgery, as described herein.

In certain embodiments the control module 124 may be used for operating one or more external systems during a mandible reconstruction surgery, as described herein.

In certain embodiments the control module 124 may be used for operating one or more external systems during a maxilla reconstruction surgery, as described herein.

In certain embodiments the control module 124 may be used for operating one or more external systems during an orbital-floor reconstruction surgery, as described herein.

In certain embodiments the control module 124 may be used for operating one or more external systems during a tumor-resection and reconstruction surgery of one or more anatomical parts of the CMF region, as described herein.

In certain embodiments the control module 124 may be used for operating one or more external systems during a trauma surgery of one or more anatomical parts of the CMF region, as described herein.

In certain embodiments the control module 124 may be used for operating one or more external systems during a craniosynostosis surgery, as described herein.

### Recording/Streaming Module 126

In certain aspects, computing environment 100 includes a recording/streaming module 126 configured to perform recording of augmented scenes. The scenes may be streamed by recording/streaming module 126 to an external audience 228 for tele surgery purposes, peer assistance and/or clinical engineering support. The external audience may interact with any virtual component of the scene to modify the augmented environment, thereby providing additional support. The environment may also be analyzed in relation to other surgeons, to provide a workflow analysis and improvement suggestions that would make the surgery more efficient. The environment may also be used for training and teaching purposes.

The augmented environment (and/or the individual virtual and physical scene components) may be recorded 234 for archiving, either as a video stream or a sub-selection of individual frames that may be taken at any point during the surgery.

Recording/streaming module 126 may record a single video stream or single frames of the entire augmented scene, and/or it may record separate video streams or separate frames for the camera view of the physical scene and/or for the corresponding view of the virtual scene. In addition, recording/streaming module 126 may record the motion data of individual objects in the physical or virtual scenes, such as tracked objects or the display device 104. Based on such data, recording/streaming module 126 may display or hide individual virtual elements as augmentation elements in their appropriate positions with respect to the recorded camera view of the actual scene at any time during streaming a previously recorded scene.

Additionally, by tracking an implant's position intra-operatively, the actual position of the implant as it is placed during surgery may be recorded by recording/streaming module 126, either to avoid post-operative imaging or to complement post-operative imaging. This data may be used to do post-operative analysis. Further, this data may be used for reporting in case of adverse events, or as part of the patient information or for retrospective studies.

In certain embodiments recording/streaming module 126 of the AR system may be used during a surgical procedure as described herein.

In certain embodiments recording/streaming module 126 of the AR system may be used during a craniomaxillofacial surgical procedure as described herein.

In certain embodiments recording/streaming module 126 of the AR system may be used in combination with one or more modules of the AR system as described herein.

In certain embodiments recording/streaming module 126 of the AR system may be used at the virtual workbench as described herein.

In certain embodiments recording/streaming module 126 of the AR system may be used during an orthognathic surgery as described herein.

In certain embodiments recording/streaming module 126 of the AR system may be used during a trauma surgery as described herein.

In certain embodiments recording/streaming module 126 of the AR system may be used during a mandible reconstruction surgery as described herein.

In certain embodiments recording/streaming module 126 of the AR system may be used during a maxilla reconstruction surgery as described herein.

In certain embodiments recording/streaming module 126 of the AR system may be used during an orbital-floor reconstruction surgery as described herein.

In certain embodiments recording/streaming module 126 of the AR system may be used during a tumor-resection and reconstruction surgery of one or more anatomical parts of the CMF region as described herein.

In certain embodiments recording/streaming module 126 of the AR system may be used during a trauma surgery of one or more anatomical parts of the CMF region as described herein.

In certain embodiments recording/streaming module 126 of the AR system may be used during a craniosynostosis surgery as described herein.

### Augmented Environment

One or more modules of the AR system may work together to generate an augmented environment that is then displayed on a display unit 104.

For augmentation elements (cf. below), different virtual objects, such as data points (e.g., measurements, patient statistics, etc.), parts of patient anatomy, surgical guides, implants, etc. may be visualized by visualization module 110 on display device 104. This may include virtual objects of the patient anatomy being shown in different states (e.g., pre-operative, intra-operative, post-operative) such as to help guide a user during the surgery.

Different states may be used separately, together, sequentially or mixed to augment the environment, depending on the information the user is looking for. Augmentation elements may belong to any one or multiple of these states.

Embodiments of the systems disclosed herein may be used pre-operatively and/or intra-operatively and/or post-operatively (e.g., to monitor patients during recovery). Augmentation elements 208 (e.g., virtual objects) are virtual data that is added to the real scene 230 to augment it. The ‘augmentation elements’ may be pre-determined based on medical images (e.g., through segmentation or post-processing of the medical image data, or as a result of a surgical planning step), or loaded from a medical device library, and/or derived from population data and/or resulting from simulations and/or detected intra-operatively with a camera system.

First, the surgical scene, e.g., the part of the actual scene that comprises the surgical window and all relevant anatomical parts, may be augmented by emphasizing or highlighting anatomical structures (e.g., with overlays or contours) by visualization module 110.

In certain embodiments for CMF surgery, such as for orthognathic surgery or reconstruction surgery, this may include anatomical elements that are not directly visible inside the surgical window such as the full bony anatomy of the patient (e.g., predominantly skull, mandible, maxilla) or soft-tissue structures inside or around the bones such as maxilla or mandible (e.g., oral mucosa, gingiva, ligaments, muscles, nerves, vasculature, cartilage, etc.). This may also include specific parts of the anatomy that are clinically relevant (e.g., for sizing, fitting) such as teeth (or dental prosthetics), cartilage, specific parts of the bone or known defects (e.g., cavities, holes). This may also include pre-existing hardware, such as implants or dental implants that were present upon acquisition of the pre-operative medical images. This may also include anatomical structures that were previously removed (e.g., either already pre-operatively due to trauma or in another surgery or during the surgery itself) and need to be displayed back by visualization module 110 on display device 104, for example the pieces of bones (e.g., osteophytes, osteotomy pieces) or dental structures (e.g., teeth, dental roots) that were removed and are visualized again on the anatomy to act as guidance for reconstructing the original situation. This may also include pieces of the anatomy that will be adapted by the surgery, for example highlighting the pieces of bone that will be resected based on the plan (parts of the bone that will be removed by the cuts). Through virtual overlay, transparency of parts of the anatomy may be modified to show the post-operative situation, e.g., by virtually making the bone portion to be removed transparent or colored up until the cutting plane or by displaying a simulation of the bones post cutting. This may also include specific parts of the anatomy that need to be saved or protected during surgery, for example (e.g., parts of) specific muscles or ligaments that will help avoid fracture or dislocation when they are spared and need to be saved or nerves or vasculature for reconstruction cases. The visualization module 110 may also highlight specific bony structures or muscles that can be sacrificed. Alternatively, a simulated anatomical situation (e.g., a reconstructed healthy bone from the actual defected bone, a healthy bone based on population data or a mirror image of a healthy contralateral side of the patient) may be visualized. Also, a bone/implant contact area may be highlighted after the initial resection, e.g., to demonstrate the region where the implant will be properly supported and where a bone graft may be required. This may be done based on a planned implant position or based on an intra-operatively captured implant position as by the surgeon who is handling the components.

Additionally, all other data or any combination of data acquired from the patient may be displayed in an augmented environment 220 as an alternative to providing external screens or printouts to display this information, e.g., through virtual or floating 2-D/3-D panels in the environment. This may include the medical images that are available-such as CT images, (CB)CT images, MRI images, X-ray images, ultrasound images or fluoroscopy images including but not limited to 2-D slices, one or more volume-rendered images, resliced images in line with the patient orientation or relevant viewing directions, etc., either acquired pre-operatively or acquired intra-operatively and shown in real time—the surgical plan as it was created, simulations of motion (or range-of-motion), predetermined implant types or sizes and instruments, screw lengths/types or general patient information (name, surgical side, etc.). This may also include running the surgical plan as a video or animation inside the augmented scene for more detailed guidance during the surgical intervention.

In some embodiments, the surgical scene may also be augmented with anatomical landmarks or structures directly derived from those anatomical landmarks. These may include individual locations in space (points, lines, curves or surface areas) such as points on the mandible, maxilla, nasal area, dentition, TMJ, etc., such as parts of the mandibular body (alveolar process, alveolar juga, mental protuberance, mental tubercle, mental foramen, mandibular angle) and/or parts of the ramus (anterior coronoid process, posterior condylar process) and/or mandibular notch, neck of the condylar process, pterygoid fovea, masseteric tuberosity, mandibular foramen, lingula of the mandible, mylohyoid groove, ptcrygoid tuberosity, mylohyoid line, submandibular fossac, sublingual fovea, interalveolar fovea, interalveolar septa, mental spine, digastric fossae, or parts of the nasal area (nose, nasal septum, piriform aperture, choana, posterior process, cartilage, perpendicular plan of the ethmoid, sphenoidal crest, vomer, cribriform plate of the ethmoid, conchae nasales, inferior concha, sphenoethmoidal recess, sphenopalatine foramen, perpendicular plate of the palatine bone, uncinate process, maxillary hiatus, ethmoidal bulla, ethmoidal infundibulum) and/or parts of the TMJ (head of the mandible, mandibular fossa, articular tubercle, external acoustic meatus, middle cranial fossa) and/or parts of the dentition (tooth, gum, tooth root, cusps, fissures, grooves, tubercula, foveac). It may also include points, lines, planes derived from cephalometric analysis such as A point (subspinale, or A), anterior nasal spine (ANS), articulare (Ar), B point (supramentale, or B), Basion (Ba), Condylion (Cd), center of face (CF) point, Gnathion (Gn), Gonion (Go), Menton (Me), Nasion (N)(, Orbitale (o), Pogonion (Pg), Posterior nasal spine (PNS), Porion (Po), Pt point (Pt) and Pterygomaxillary fissure (ptm). These may also include lines or planes derived from anatomical landmarks, such as lines that represent either anatomical or mechanical axes of individual bones or limbs, lines or planes that represent an anatomical coordinate system (e.g., an axial, coronal and sagittal plane) or resection lines.

In some embodiments, the surgical scene 230 may also be augmented with information that represents a mechanical, physical, anatomical or other feature (which may be mapped onto a surface, e.g., as a color map) and which is derived from a calculation or a direct measurement 232 based on medical imaging or pre-operative or intra-operative data acquisitions. Examples include bone quality, e.g., derived from greyscale values in medical images, soft-tissue thickness maps, derived from visible coverage in medical images or virtual 3-D models, or skin thickness or elasticity maps, derived from imaging measurements, palpation or simulations, or color maps on the teeth to indicate the degree of grinding necessary to achieve a planned occlusion. These may be visualized as a color map overlaid on the anatomy. Another example is a simulation of the post-operative range-of-motion which may be visualized as trajectories, lines, zones or as a video, animation or dynamic 3-D model. A virtual skull or representation thereof may be used to simulate the range of motion of a TMJ. The interaction of the AR system with the skull allows the system to transfer to the augmented environment where the motion is simulated as a virtual model overlaid/floating above the real location to enable the surgeon to achieve the optimal post-op results. Post-operative range-of-motion may be predictively simulated via (musculoskeletal or other) modelling.

In some embodiments, the AR system may be used to show the assembly of multi-component implant systems, e.g., by demonstrating how they need to be assembled, or by allowing to assess the quality of the assembly afterwards.

In some embodiments, the AR system may be used to show the modification of an implant system, e.g., by demonstrating how it needs to be bent, expanded, cut, assembled, or joined together, or by allowing to assess the finished modification afterwards.

In some embodiments, planned or simulated instrument trajectories, positions, orientations and/or locations 216 may be visualized as part of the augmented environment. This may include drilling or screwing trajectories (e.g., to insert fixation screws for an implant or to predrill the holes for such fixation screws, to insert dental implants or to predrill the holes for such dental implants), reaming trajectories, biopsy trajectories, cutting planes (e.g., for directly guiding osteotomies or resections, such as tumor resections). All of these instrument trajectories have a location and orientation in space that is linked to the anatomical part on which the instrument is to be used. The visual elements representing the instrument trajectories are displayed as objects in 3-D space of which the locations and orientations are correct with respect to the user's view of the relevant physical anatomical part and follow any movement of that anatomical part through the user's field of view. Drilling, screwing, reaming or biopsy trajectories may, for example, be visualized by visualization module 110 on display device 104 as solid, dashed, or dotted lines or line segments, as arrows, or as elongated 3-D shapes-such as cylinders or prismatic shapes—optionally with a diameter correlating to the diameter of the drill bit, pin, or screw. Cutting planes may, for example, be visualized as planar shapes-such as polygons, circle segments or fan shapes—or as very thin 3-D shapes-such as flat prismatic shapes or segments of thin disks-optionally with a thickness correlating to the thickness of the cutting blade. All of these objects may be visualized in any color, but preferably colors that contrast—for example in hue or luminosity—with the background. They may be shown in various degrees of transparency or fully opaque. They may be visualized with or without taking occlusion into account, as described above. For example, only the part of a drill trajectory that lies outside the bone may be shown. Optionally, parts of these augmentation elements may be visualized differently depending on whether they are outside or inside of the anatomy, e.g., by changing color, transparency or texture. The augmentation elements representing instrument trajectories may comprise a depth indication to indicate the planned depth to which a drill bit, sawblade, reamer or biopsy needle should be inserted into the anatomy. The depth indication may, for example, be a line or plane perpendicular to the trajectory at a distance from the surface of the anatomy equal to the length of the drill bit, sawblade, reamer or biopsy needle reduced with the planned depth. When the proximal end of the actual drill bit, sawblade, reamer or biopsy needle reaches the depth indication in the enhanced scene, the user knows that the planned depth has been reached.

The scene may also be augmented with parameters from the instruments used during surgery. This may include drills, saws, plate-shaping instruments (such as bending pliers, plate cutters, bending irons), reamers, etc. Besides location and orientation parameters, parameters can include drilling speed, drilling torque, drill or saw temperature, bending angle, bending torque, etc. These may be visualized as numbers or by adapting the visualization of the drill (e.g., based on the drill temperature) or the bone (e.g., based on predicted bone necrosis at the real-time drill temperature) or the plate (e.g., based on angulation of the bent required).

Additional screw parameters may also be visualized. These can include force or torque measurements acquired using the screwdriver, representing the quality of fixation of a specific screw (or allowing the comparison of fixation between different screws).

Other relevant information such as vital signs such as heart rate, breathing patterns, anesthesia status, anatomical regions to avoid such as locations of nerves, blood vessels and teeth, etc., of a patient may be visualized.

Alternatively, or additionally, instrument trajectories may also be visualized as insertion locations (entry or exit) on the anatomy, such as insertion points for drills, screws or pins, or cutting lines projected on the surface of the anatomical part to be operated on. These may assist the surgeon in freehand guidance. This may also apply to the initial incision (e.g., to open the anatomical region of interest) where minimally invasive (or otherwise optimized or planned) cutting lines are overlaid on the patient skin.

In some embodiments, the geometric alignment of a tracked instrument, such as a drill, reamer, or biopsy needle, to the entry point location on the bone may be visualized by changing the color of a virtual point (e.g., from red to green when the Euclidean distance is smaller than a predefined threshold).

In some embodiments, for angular orientation of instruments (e.g., a drill, reamer, biopsy needle or saw), the user may be guided, e.g. by changing the color of the augmentation element representing the trajectory in accordance with the difference in angulation between the instrument and the planned trajectory, or by displaying a target point on the same line as the planned trajectory, but rather than on the entry point of the drill, shown either more proximally or more distally to the user on this same line and changing the color of this virtual point (e.g., from red to green when the angulation error is smaller than a predefined threshold).

In some embodiments, the placement of an implant, such as a plate, on the bone with respect to predrilled holes in the bone for fixation elements, such as screws, that will fixate the implant on the bone may be visualized by showing the virtual plate or an outline of the virtual plate against the drilled holes.

All of the above may not only apply to acts to be performed on bony anatomy, but also to acts to be performed on other tissues, such as muscles, fat, skin, organs, etc. Further, it may also apply during the process of adaptation of a medical device such as plate shaping (e.g., bending).

In some embodiments, the surgeon may wish to use virtual elements 208 in the augmented environment 220 corresponding to or relating to physical objects that he/she plans to place or use in the actual scene during surgery. This could include elements such as virtual guide wires, flags, annotations, surgical tags, etc., and may serve as an intermediary support tool during surgery or as reference markers for post-operative analysis. These could also be instruments such as catheters or endoscopes whose position may be measured or simulated and visualized in the augmented environment.

In some embodiments, virtually calculated components that may facilitate part of the surgical workflow may also be used to augment the environment. As an example, the surgeon may want to include grafting guidance, e.g., for fibula or scapula or hip or synthetic or allografts or xenografts. These grafts can be manually shaped based on a virtually calculated template shape that is displayed in the augmented scene and can visually (or otherwise) be compared to match the shape of a harvested graft to the template (e.g., at the virtual workbench as described herein). As another example, the surgeon may use one or more virtual pins and/or virtual planes for reaming or burring guidance. A virtual plane may be visualized at the optimum reaming or burring depth. One or more markers and/or sensors may be used for guiding the reamer or burr. Virtual labels may also be used for marking the optimum reaming or burring depth on the virtual plane and/or the reamer or burr. Reamers or burrs may also be used for bone and/or teeth smoothening and shaping, etc.

In some embodiments, intra-operative data or simulated metrics may be visualized as well, either as number, figures, graphs, etc. These may include data such as alignment of maxilla to the mandible, predicted post-operative occlusion, predicted post-operative range-of-motion, predictive post-operative appearance of soft tissue (e.g., soft-tissue simulation in the PROPLAN software), etc. This may also include simple metrics such as surgical duration (time), blood loss, vital signs, etc.

Specific validation or quality-control elements may also be added to the scene 220. An example could be a workflow to intra-operatively validate a pre-operative plan based on a series of intra-operative steps where indications may be given that a plan needs to be adapted and how, based on any complications encountered. Alternatively, in cases of trauma or emergency situations, surgery may be adapted due to lack of available medical devices and the surgeon may only have a handful of standard medical devices at his/her disposal.

For all augmentation elements, a library of pre-computed (or real-time adapted) options may be used to browse through. These may include multiple implant types including different sizes, thickness, variations in fixation holes (e.g., standard plates of varying shapes) and may include different or the similar planned positions (‘library of plans’). Access to such a library may be limited during surgery to only show the options that remain based on the anatomical regions, the type of surgery, the progress of the surgery and the surgical choices that were already made, providing step-by-step guidance.

Alternatively, access to such a library may be limited based on availability of medical devices. For example, looking at a zygomaticomaxillary complex (ZMC) fracture may restrict access to the appropriate size and shape plates to stabilize the various fracture points, such as maxillary buttress, zygomatic arch, ZF suture, orbital floor, etc.

Any snapshot of the surgical procedure as executed or as planned may be added to the library during or after surgery for reference.

Alternatively, a library of medical devices may only be available for browsing whilst highlighting the available options. These may include instruments, multiple sizes, or implant times (time in surgery for shaping and/or placing an implant based on the chosen implant and/or surgical approach) e.g., in case of time-sensitive cases such as trauma or emergency surgery wherein the surgeon only has a few instruments and implants or plates available to his/her disposal, providing step-by-step guidance.

All augmentation elements occupy a position, e.g., a location and orientation, and have a scale or size in 3-D space. Location, orientation and/or scale/size may be fixed relative to any part of the scene, such as the world coordinate system (e.g., the scene itself, the operating room), (part of) the patient, (part of) an instrument, implant or implant component, or the user's field of view. When that part of the scene moves through the world coordinate system, the system automatically tracks the movement, such as by I/O module 122, and updates the augmentation elements' locations, orientations and/or scales, such as by visualization module 110, accordingly in real time. The system also constantly tracks the position of the user's display unit, derives from that position the user's field of view, and displays by visualization module 110 within the display unit 104 all relevant augmentation elements within that field of view.

Some augmentation elements are inherently linked to an object that can move in the scene, such as an anatomical part of the patient, an actual instrument, actual implant or actual implant component. For example, an augmentation element representing a planned implant component may be displayed in its planned position with respect to a particular anatomical part of the patient in a scale of 1:1. As the anatomical part moves with respect to the world coordinate system, the virtual representation of the planned implant component follows that movement, so that its position relative to the anatomical part stays the same. Similarly, an augmentation element representing the final shape of an implant to be shaped (e.g., bent or cut) may be displayed on the standard implant in a scale of 1:1.

In some embodiments, an augmentation element may comprise one or more virtual 3-D models of the standard implants (also known as a virtual template) that is linked to one or more physical standard implants (plates). This enables the user to shape (e.g., bend) the plate in real time by following virtual guidance (e.g., angulation, bending trajectory) displayed on the virtual template. As the user shapes (e.g., bends) the plate as per the guidance, the virtual implant also changes in shape to resemble the physical plate thereby allowing the user to verify the shaping (e.g., bending) simultaneously. Alternatively, or additionally, similar augmentation elements may be used for expanding a plate by joining one or more standard plates (like pieces of a puzzle) and/or used for guiding cutting to make the plates smaller. For example, when the surgeon has only limited medical devices to work with, he/she may have to adapt more than one plate to correct a deformity/fit a patient. In this case, one or more plates may be cut, bent, or reshaped otherwise to form one, homogeneous (expanded) implant.

Intra-operative data, optionally, such as numbers or graphs, may be positioned in a fixed position relative to the world coordinate system: for example, on a virtual plane that has a fixed position in the operating room. As the user moves through the operating room, it appears as if the intra-op data floats at a certain position in the room, as if there were a computer display unit positioned there, but with the advantage of not having a physical computer display unit occupying space in the operating room. The system, such as via I/O module 122, may provide ways for the user to interact with it to change location, orientation and/or scale of the displayed elements, for example, by means of gesture-based controls.

Intra-operative data may also be displayed by visualization module 110 on display device 104 with a fixed location but a variable orientation relative to the world coordinate system: for example, on a virtual plane that has a fixed location, e.g., center point, in the operating room, but automatically orients itself towards the user.

Alternatively, intra-operative data, such as numbers or graphs, may be positioned in a fixed location relative to the user's display unit. As the user moves through the operating room, the intra-op data will remain in the same location in his field of view. In certain embodiments, such data will occupy positions in peripheral areas of the field of view.

Certain intra-op data with a particular relevance for an object in the scene 220 may also be displayed in a fixed location and/or orientation relative to that object. For example, relevant data of individual teeth or (e.g., part of) jaw or nerves may be displayed as callouts attached to the respective tooth or nerve. The distal end of the call-out's pointer may have a fixed location with respect to the relevant tooth or nerve. The location, orientation and/or size of the call-out's data field may be automatically updated by the system for optimal viewing. For example, all the callouts visible at a given moment in time may be distributed over the field of view so that their data fields don't overlap, so that their pointers don't cross and/or so that their data fields don't obscure the view of the relevant anatomical parts. For example, any text or numbers in the data fields may be displayed in the user's display unit with a constant font size or with transparency, irrespective of the relative positions of the callouts and the user's display unit.

The position of an augmentation element can also be determined per degree of freedom. Individual degrees of freedom may be linked to different parts of the scene or the user's display unit. For instance, an augmentation element that represents a set of intra-op data, such as a graph, might be a virtual plane on which the graph is shown. The position of the augmentation element may then be expressed as three spatial coordinates X, Y and Z of an origin point of the virtual plan in the world coordinate system, and three angles αₓ, α_{y} and α_{z} representing the orientation of the normal vector of the virtual plane and the roll angle of the virtual plane around its normal vector with respect to the world coordinate system. Each of these six degrees of freedom may then be locked on to different parts of the scene or the user's display unit. For example, X, Y and α_{z} may be locked onto the user's display unit 104, such that the virtual plane is always directly in front of the user and rotated towards the user, while Z, αₓ and α_{y} are locked onto the world coordinate system, such that the virtual plane remains vertical and at a certain height.

As described above, in a multi-user system, it may be possible for each user to view a different selection of augmentation elements. For example, users may select which augmentation elements to view at any given moment on a corresponding, personal display device 104. Alternatively, an automatic selection may be made by the system dependent on the role of the user (surgeon, assistant, anesthesiologist, nurse, etc.) and/or the stage of the procedure. The system may be pre-configurable in this respect to best accommodate the surgical staff's preferred way of working.

In a multi-user system, locations, orientations and/or scales of some or all augmentation elements may be configurable or optimized per user. For example, certain intra-op data may be displayed oriented towards each individual user or sized per individual user. Other augmentation elements may be visualized to more than one user in the same location, orientation, and scale, such that users can concurrently look and point to the augmentation elements while discussing the details of the case.

In certain aspects, for better visualization, some augmentation elements have the functionality of magnification. For example, during plate shaping (e.g., bending), as it is important that the shaping (e.g., bending) is guided perfectly, the user may benefit from added functionality of zooming in so as to achieve effective shaping (e.g., bending), and subsequently zooming out to verify the overall effect. This may be achieved by capturing the video feed of one or more cameras in the user's display device 104, such as an OHMD, applying identical magnification to the video feed and to the augmentation elements, and displaying both the magnified video feed and augmentation elements in the display device 104 (in the case of a see-through device effectively blocking the full view through the see-through device). The user may freely choose the magnification factor. This functionality compares to the user wearing magnification glasses.

Next to the display of virtual elements that can serve as a visual guiding tool during surgery, more explicit guidance 214 is also possible with augmented reality.

One way of achieving this is by tracking the positions of instruments (e.g., drills, sawblades, reamers, pliers, etc.) within the scene and analyzing the alignment between an instrument and its pre-planned trajectory (e.g., drilling path, saw planes, reaming path, bending path, expanding path, cutting path, etc.). For example, in orthognathic surgery, e.g., Le Fort osteotomy, the AR system may suggest to the user the type (I, II, III) of the Le Fort osteotomy suitable for a particular surgery. Alternatively, the AR system may produce direct parameter differences such as angles between planned and actual instrument trajectories and distances between planned and actual locations of surgical acts—e.g., distance between planned and actual entry points of a drill, difference between planned and actual cutting plane and length, etc.—, it may produce derived quality metrics that represent the instrument alignment (‘cut quality’) or it may produce resulting clinical parameter error, such as relating to dental occlusion, pitch, roll, yaw etc. Any of these parameters 216 may be visualized by visualization module 110 on one or more display devices 104 to one or more users by means of the augmentation elements described above. Based on these parameters, warning signs, such as visual and/or audible and/or tactile signals, can be given to the surgeon. For example, the color or luminosity of a visualized planned instrument trajectory may vary dependent on the difference between the planned and actual instrument trajectory. Additionally, or alternatively, suggestions for improved instrument alignment could also be derived by the AR system and provided to the user by visualization module 110 on one or more display devices 104. For example, a straight arrow may be displayed between the tip of a drill bit and the planned entry point, or a curved arrow may be displayed between a drill and its planned trajectory to indicate how and how much the angulation of the drill should be adjusted. This may also be visualized for any other medical device such as a plate.

Creating alignment of instruments to a planned target, e.g., aligning a drill with a planned drill position and orientation, may be split up in multiple stages or displayed via several augmentation elements, e.g., by incremental registration performed in stages as described above.

The analysis that is the basis for suggestions made by the AR system may also be based on a range of plans and thereby aim to control only a single parameter, such as the Le Fort cutting plane angle (but not the cutting depth). It may use a range of acceptable locations to provide a safe zone within which no further guidance is needed. This safe zone would include all acceptable cut positions within a desired clinical objective. It could be displayed as a fan or cone instead of a single plane to indicate to the surgeon which are acceptable sawblade positions or by coloring the regions that may not be cut. This region could be visualized differently (e.g., in size or color) depending on the position of the instruments in relation to the optimal or planned position.

Next to guidance of individual surgical steps by guidance module 116, the AR system can also be used to guide users through the surgical workflow. The surgical steps may be detected automatically, e.g., by tracking time, detecting voice commands or analyzing conversation patterns, visually/audibly/etc., tracking instrument usage (including drills, saws, implant components, guides, etc.), identifying and classifying the surgical scene or surgical window based on machine learning or other trained methods or a combination of the aforementioned. AR guidance may include providing, by guidance module 116, the right data at the right time, automatically (e.g., by detecting the surgical step and providing the information that belongs to this surgical step) or semi-automatically (e.g., as activated through voice or other input controls by the surgeon or his/her staff). It may also demonstrate appropriate instruments to use (screws, surgical instruments, etc.), their use thereof, and highlight these in the operating room. For example, a halo may be displayed around the (physical) next instrument to be used. It may allow the surgeon to find these instruments more easily after indexing the operating room by directing the surgeon's or surgical staff's attention to the instruments through any guidance means, be it directional guidance (arrows), refocusing of the scene, or others. This information may also be provided to other members of the medical staff such as a surgical nurse present in the OR. It may also assist the surgeon during adaptation of certain medical devices (e.g., plate shaping (e.g., bending)). This may also include keeping track of the instrument inventory such as surgical trays and their contents, such as the currently available implant or screw inventory at a specific time point during surgery.

The system may thereby also be used to automatically track instruments and their usage, e.g., by directly recognizing them from a video stream or by attaching or embedding a marker system on the instruments. An example could be to track screw lengths with a (e.g., bar/QR) code scanner integrated into the system and/or using color-coded markers (or sensors) for identifying instruments. An example could be to verify the inventory against the standard medical devices (e.g., plates, screws) available in the OR and highlight them on the virtual inventory. Recognition may be done by using standard tracking devices such as QR codes, markers or a simple picture taken using one or more embedded cameras.

For invasive acts, such as cutting, drilling or reaming, it may not be advisable or even allowed by law to obscure the area of the anatomy where the invasive act is to be performed by some augmentation elements. The system may therefore detect the handling of an invasive instrument, such as a drill, reamer or saw, and automatically make it impossible to display such (obscuring) augmentation elements as long as the instrument is being operated. For example, at any given stage of the surgery, the system may detect the user picking up or activating a drill and may automatically hide visual representations of any planned implant components that would obscure the part of the anatomy in which the surgeon is to drill at that stage of the surgery.

During surgery, certain actions may cause damage to tissue structures or may lead to unwanted side effects after surgery. In certain aspects, computing environment 100 may give feedback via the visualization module 110. The feedback may include warning signs for display by visualization module 110 through display device 104, or through I/O module 122, to prevent those. Such warning signs can include visual, audible or haptic feedback. For visual feedback, the alerts may be provided by highlighting specific parts of the scene. This may include highlighting instruments or anatomy (using colors, illumination or other means) to stop the user from advancing the instruments any further. Alternatively, the alerts may trigger a broader response on the entire field of view, for example by coloring it in a specific shade. Alternatively, a traffic-light system may be implemented.

For example, a warning sign may be given while performing an osteotomy, where based on the sawblade location an estimate is made of the risk of damaging soft-tissue structures such as the nerves, blood vessels, muscles, or sensory organs, etc.

The warning signs may be linked to guidance elements that give suggestions on improving component position or planning, e.g., increase/decrease osteotomy length, increase/decrease angulation, etc.

A warning sign may also be displayed to alert the user for rinsing/sanitizing of instruments or anatomy (e.g., on pre-determined time points to reduce the chance of infection or heating).

A warning sign may also be given to alert the surgeon when he has deviated from the pre-op plan such as when he is not using the same devices as those that were planned. This could be done by detecting the implant components or fixation elements that the surgeon is using and comparing them to the pre-op plan.

The augmented reality system may interact with other physical guiding systems such as patient-specific or generic cutting guides/blocks, robots, or other systems, such as via I/O module 122 or those integrated into the AR system and operated via the control module 124.

The versatility of the AR system may allow the user to use it with existing technology such as patient-specific guides and/or implants. A surgeon may prefer to use a patient-specific guide and/or implant but to have more information available to him/her in the OR. The AR system may recognize the guide and/or implant by means of any shape or feature-recognition techniques known in the art, and may display relevant information about the guide and/or implant or the underlying bone, display trajectories of instruments that would interact with the guide, mark the position of the guide, etc. The guide and/or implant itself may contain specific landmarks (such as mechanical axis, landmarks, etc.) that are aligned with the augmentation elements in the augmented scene, e.g., for aligning the guide and/or implant, registering the scene or for quality control motivations.

A patient-specific guide system may also be used as a reference marker for the AR system. Based on the unique fit of a patient-specific guide system, it may immediately initialize the registration, by registration module 114, either by attaching a marker to the guide or by using the guide itself as a marker. Also, by tracing the contour or specific features of the guide with a pen or guided marker, registration 218 may be performed.

For traditional patient-specific guiding systems, a unique, easy and stable fit to the anatomy is required. Using AR, this may be improved. First, the AR system may increase the ‘easiness’ of finding the fit with the anatomy e.g., by highlighting the guide outline before placement. Further, using the AR system, a unique fit is no longer a requirement as the AR system can determine whether the guide is in the correct position, even if it could possibly fit in multiple positions. Also, the AR system introduces the possibility to reduce a guide's footprint by not having to look for anatomy that would make the fit unique, which is beneficial to reduce the incision and the case of use for the surgeon. Also, combining the AR system with a patient-specific guide may allow creation of a stable guide based on less information, e.g., by performing the design on lower-dimensional data (X-ray instead of 3-D imaging) and correct for potential variations introduced due to the sparsity of the 2-D information with intra-operatively acquired AR information. Additionally, some features may be eliminated in the guide system and replaced by augmented elements in the augmented scene, e.g., the drill cylinders for guiding entry point and orientation of the drill could be reduced to entry-point guide elements combined with augmented elements for drill orientation guidance. Adaptive guide systems (which may be patient-specific) may also be configured, modulated or set up using the AR system. For example, this may include guide systems that have multiple contact surfaces to modify an angulation or seating position, where the AR system can show the correct position or configuration of the adaptive guide. In another example, this may also include providing guidance on the use of specific cutting slots or drill barrels, where multiple options may be provided by the guide system and the appropriate option is highlighted using the AR system. In another example, this may also include providing guidance for harvesting of a bone segment (e.g., fibular) by showing correct location and position for performing osteotomies (e.g., an L-shaped guide designed on the basis of a generic fibula model). Further, the combination of the guide system and the AR system allows the user to access anatomical regions that may be difficult to reach, thereby providing a holistic picture of the surrounding anatomy.

In some embodiments, the AR system, such as via guidance module 116, may interact with standard or adjustable instrumentation to provide the right settings for adjustable instruments (such as angles, lengths) based on the surgical plan. These settings may be displayed on and/or around the instrument itself. For quality assurance reasons, the settings of the instruments may be automatically detected using the camera system to validate. Based on the actual settings of the instruments, the plan may be recalculated automatically. An instrument's position may be tracked automatically in relation to the bone (either using a marker attached to or incorporated in the instrument or using the shape of the instrument itself as a marker). The location of instrument guiding elements (e.g., cutting slots, drilling holes) on the instruments may be virtually augmented with cutting planes, drilling lines, any other guidance mechanism, etc. These virtual elements can directly interact with the anatomy of the patient and adapt the visualization of this anatomy, as described earlier. The system may track the instrument stock in real time, for example, the number and type of screws already inserted in the patient. This may be tracked by using optical recognition to recognize the implant and/or the instrument or their packaging or (parts of) their labeling.

In some embodiments, the I/O module 122 for example, may be able to interact with a robot system, e.g., by actively positioning a robot arm or providing instructions for manual positioning of the robot arm, e.g., by showing a target position or providing force-based guidance to the robot. In some embodiments, the system is used to control the robot, e.g., by integrating the robot control user interface in the AR system. The warning signs, safety zones or other controlling features in the AR system may also directly control the physical guidance system, e.g., by controlling the drilling, or sawing speed (modulated or a simple on/off switch).

In certain embodiments, users can interact with the system via a series of explicit or implicit commands, such as via I/O module 122. Explicit commands may include one or more of voice control, e.g., spoken commands captured by the system through a speech-recognition module, gestures captured through gesture tracking, or touch-based commands such as pressing a button, a pedal, a touch screen, or (haptic) controllers coupled to I/O module 122. Implicit commands refer to actions of the user that automatically lead to a certain system behavior. For example, head motion tracking, eye tracking or gaze tracking, etc. may all instruct the system to display, hide or alter the position or scale of certain augmentation elements. One or more combinations may be used.

In some embodiments, the system is customizable by providing one or more options to augment the real scene with virtual objects (environments) and allowing the user to control the interaction between the physical scene and the augmented environment by providing an on/off setting to switch between environments.

In some embodiments, the system may detect the surgeon's gaze using eye tracking or gaze tracking. This allows the system to either create focused visualizations or to clear virtual elements from the augmented scene for better visualization of the scene. Using artificial intelligence or machine-learning techniques, the system may be trained to perform this function.

Although in some embodiments the main function of the system is to assist the surgeon in the OR, the system when coupled with smart health devices (such as smartwatches, smartphones, tracking devices, etc.) may also be used to track health of the patient post-surgery, and based on the information gathered and by comparing it with stored patient data, the system may provide assistance/guidance to the patient during recovery by displaying exercises in an augmented environment and tracking patient movement to gather information regarding soft tissue balancing, range of motion, flexibility, post-op care, etc.

One form of interaction with the system is indicating, such as via I/O module 122, certain points in the scene, such as anatomical landmarks. In some embodiments, indicating points on the anatomy can be performed by using a tracked pointing device, such as a stylus (e.g., an input device coupled to I/O module 122). In other embodiments, points may be indicated without using a pointing device. For example, after entering into an “indicate point” mode, a target symbol, such as cross hairs, may be displayed to the user in the display unit. These cross hairs may then be moved by head or eye movement, until they point to the desired anatomical point, at which time the user can give the command to lock the position. In yet other embodiments, such a target symbol may be moved manually by the user. In such embodiments, the system may detect hand or finger movements, e.g., in the user's field of view and translate those movements to movements of the target symbol. By varying the speed at which the target symbol is moved relative to the speed of the hand or finger movements, the system may allow anything from rough indication to fine-tuning.

As the augmented reality system is able to determine the position and location of anatomy and instruments, it may be used to perform measurements intra-operatively. These measurements may influence the planning procedure or be used to display specific information.

In some embodiments, a measurement may be performed that involves the parameters of an osteotomy based on a saw blade position. By tracking the osteotomy as it would be performed with the sawblade in a particular position, the augmented scene can be adapted to display the effect of this sawblade position on the patient anatomy. This can be done by extracting anatomical parameters from the sawblade position in relation to the virtual model (e.g., calculating the effect a cut at a specific position would have on the virtual model). Such parameters could include amount of bone resected (volume, distance, resection level, angulation, etc.). These parameters could be converted to a clinical parameter such as assessment of required recut to create a wedge in case of an impaction. This may provide implicit guidance, for example for surgeons who chose not to plan the case but still want additional anatomical parameters to work with. Other examples of measurements that may be performed using the AR system are described herein.

Also, the AR system may be used to track the relative position of the individual bones or bone portions in the skull to determine their relationship to each other in a post-op scenario or compare their spatial relationship to a planned relative position of one to the other.

In another embodiment, anatomical landmarks can be manually annotated using a tracked device or pointer or by visually detecting the landmarks through the camera. From these anatomical landmarks, secondary measurements can be derived.

The augmented environment may also be used to keep additional staff informed on surgical decisions/status of surgery so they can prepare themselves better, for example by highlighting the next instrument that will be needed or the specific screw (type, diameter and length) that should be taken out of the inventory. The augmented environment may also be stored for future references wherein it can later be played as a movie, as needed. The system also enables the surgeon to pause, play, capture any part (s) of the augmented environment in the form of snapshots and/or live motion pictures at any time during its use.

The AR system may also be used to provide anesthesia guidance for patients, e.g., by showing breathing patterns or other types of guidance.

### Virtual Workbench as Part of the AR System

Certain aspects of the disclosure also provide for systems, methods, and devices of providing a virtual workbench for use in a (e.g., sterile) environment such as during an augmented-reality-assisted surgery for assisting a surgical process or parts of the surgical process.

Certain aspects of the disclosure also provide for systems, methods and devices for virtually working at one or more dedicated, localized, locations in a (e.g., sterile) environment. The systems, methods and devices relate to a user interface that seamlessly integrates the virtual world into an operating room(s).

According to some embodiments, systems and methods described herein provide a virtual workbench that may include a platform (or an interface) for interacting with by the user to provide visual guidance/assistance for planning, designing, operating using augmented-reality technology during a surgical procedure.

Certain aspects of the disclosure also provide for systems that generate a three-dimensional, virtual workbench where a user performs plurality of types of actions such as planning one or more surgical processes or parts thereof, designing one or more surgical processes or parts thereof, controlling other systems or performing one or more surgical steps simultaneously or at known intervals in accordance with the surgical procedure. Unlike conventional methods, the systems and methods described herein provide improved ability for the surgeon to plan, visualize, and evaluate surgical procedures resulting in improved patient outcomes and/or operational efficiency gains for the physician (e.g., time, logistics, etc.). Further, the systems and methods provide a virtual environment by providing access to relevant information at a dedicated location via a virtual workbench to the user thereby increasing the adaptability and efficiency of the system Additionally, the systems and methods described herein provide the user access to operate other external systems that are integrated in the AR system network such as an additive manufacturing device, such as 3-D printer to manufacture one or more components on the fly such as one or more instruments (e.g., guides, implants, screws, plates, etc.), anatomical models, other miscellaneous items that may be useful during surgery such as surgical tags, etc., robotic systems (or arms). The systems and method provide improved accuracy in surgical procedures as compared to traditional systems, again improving patient outcomes and the field of medicine by providing a dedicated, one stop, virtual workbench where all the information is available in an organized, user-friendly platform.

According to some embodiments, systems and methods described herein provide a virtual workbench. The virtual workbench may include a platform (or an interface) for the user to interact with and to provide the user visual guidance/assistance for planning, designing, operating using augmented reality technology during a surgical procedure.

An AR system may be used to perform several functions during the preparation, execution or follow-up of a surgery, such as a CMF surgery. To effectively use an AR system as a surgical-assistance tool, current interfaces are not suited as they lead to information overload, cluttering of the (virtual) operating room (theatre), complex user navigation to obtain the right functionality at the right time.

What is needed are AR systems that are more user-friendly and provide decluttered, minimalistic, simple, localized virtual environments to the users in sterile operating rooms.

Accordingly, certain aspects herein provide an improved AR system that provides seamless integration of the physical and virtual environments by providing a virtual workbench (VWB) for use in (partially) sterile environments such as operating rooms of hospitals.

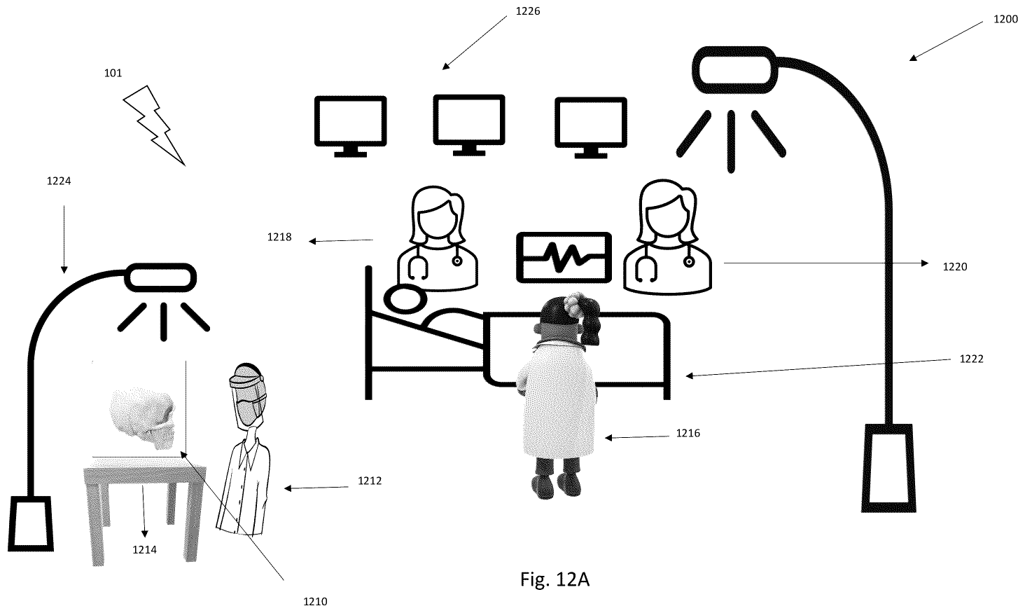

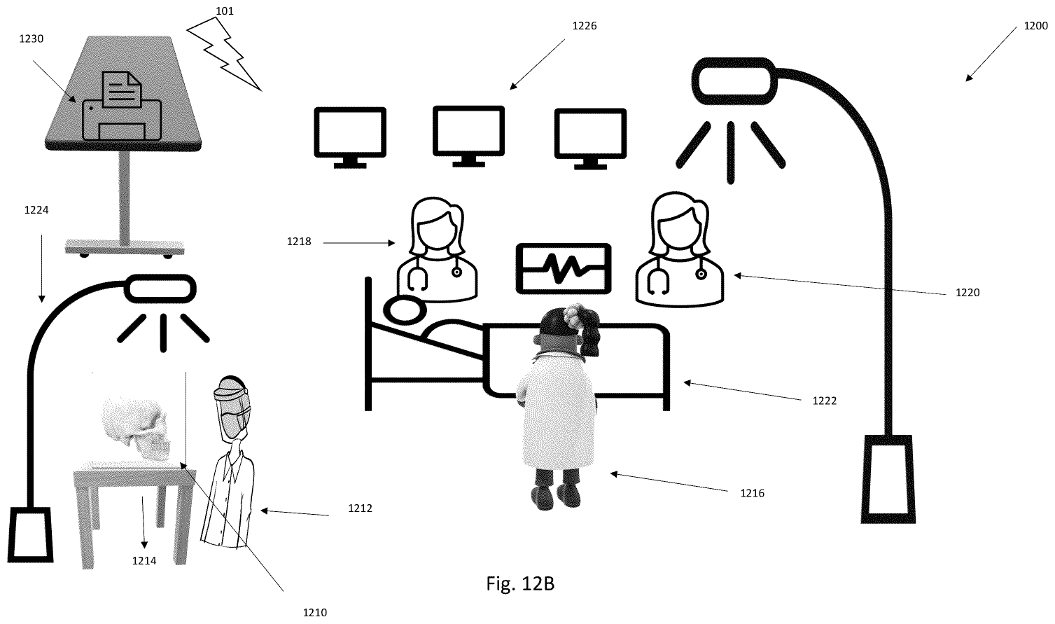

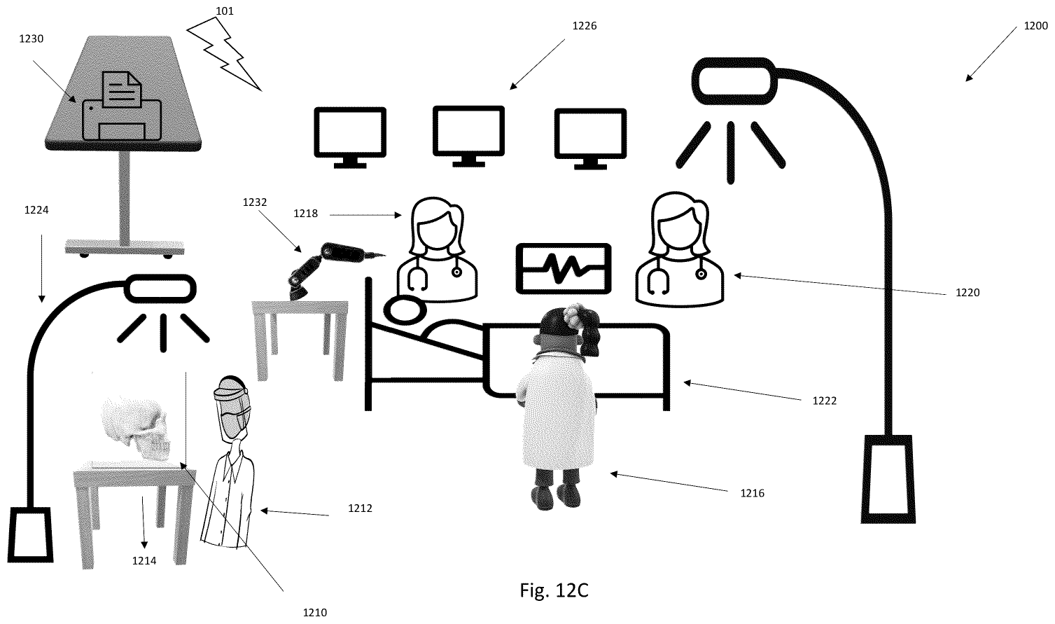

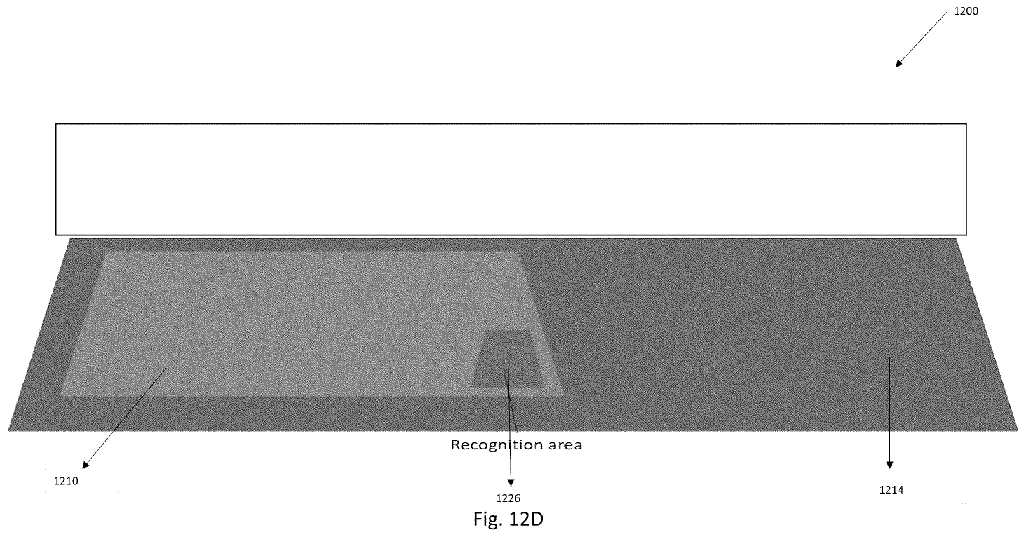

A virtual workbench is a virtual representation of a physical workbench (also sometimes known as a utility toolbox) that occupies three-dimensional volume in space (e.g., virtual space). It is overlaid on the actual environment at a desired location using augmented reality technology. It may be represented in any geometric form that occupies 3-D space, such as a rectangular, cube, cuboid, or a square virtual workbench. All the modules and components that make up an AR system are accessible virtually using the virtual workbench as described herein. A virtual workbench comprises of a virtual toolbox and a virtual platform to access said toolbox. A virtual toolbox comprises plurality of virtual tools (such as virtual template of an implant, virtual representation of a drill bit, virtual screws, virtual scissors, virtual copy-pasting tool, virtual magnification tool, etc.) that one may need during the performance of a surgical procedure, preferably a craniomaxillofacial surgery such as orthognathic or reconstruction surgeries. A virtual workbench seamlessly integrates and connects the physical and the virtual environment, as shown in FIG. 12A. Access to a virtual workbench is enabled by a simple, minimalistic platform (or a user interface) via the user's OHMD device as described herein.

For example, in certain aspects, a virtual workbench facilitates the interaction of a user with an AR system and seamlessly integrates the virtual, augmented world into the physical, actual world. It may be an extension to a physical operating room (theatre) with an interface to access an AR system. A VWB provides the user with a single-entry point to an AR system, its data and its modules. Similar to a physical workbench that comes with a toolbox, such as an artisan's workbench with tools, a virtual workbench provides a user access to virtual tools which are part of the AR system and which may be called upon to execute one or more tasks during a CMF surgery.

In certain aspects, a virtual workbench organizes the access to data in an AR system in a relevant way in space (e.g., intelligently selecting the location of virtual elements in the OR), function or time (e.g., modifying the interface of the AR system according to the task a user is performing with an AR system).

In certain aspects, a VWB may be accessed through any head-mounted or portable electronic device, mobile device or other display systems which provides augmented reality capabilities.

According to some embodiments, virtual workbench provides a user the functionality to design, plan, guide/assist, control other systems, etc. During an augmented reality assisted surgery.

According to an embodiment, the systems and methods provide a virtual workbench during an augmented reality assisted craniomaxillofacial surgery, in particular orthognathic and/or reconstruction surgery.

According to some embodiments, the seamless integration of the virtual and the physical environment in an operating room enabled by the virtual workbench is shown in FIGS. 12A-12C, and described herein. The virtual workbench is available to all the users that have access to the AR system 1200 via an OHMD device (not shown).

Various modules of the AR system such as the registration module 114, the planning module 108, calibration module 112, display module 104, virtual 3-D model creation module 106, etc., relay the information to the virtual workbench bench where it is accessible at all times to all the connected users of the AR system, making it easily available at one location instead of providing it on numerous, floating virtual screens.

### Activation of the Virtual Workbench

The virtual workbench may be accessible via a visual marker (e.g., QR code on an object such as a surgical tray or printed on paper) or via a virtual button on a display device that can be activated by looking at a visual cue or by performing an action in the interface of the AR system. It may also be activated through a voice command, hand gestures or eye gaze, etc. Once the user has selected the location in their physical environment for the virtual workbench, it is activated. The user may select the location based on personal preference either by physically moving the visual cue to the desired location or by virtually fixing the workbench to a location in space, such as proximity. The AR system prompts the user to confirm the location of the virtual workbench. Once confirmed, the virtual workbench is then virtually fixed in space at the selected physical location using spatial understanding. Various methods of spatial positioning are known in the art. For example, the user finds a less often but still easily accessible spot near the surgical table or tray in the OR. For example, a QR code may be located on a surgical tray or table close to the user. In some embodiments, the virtual workbench may be accessible when the user is standing facing the location or from a distance. As it is virtual, it does not take up any physical space in the OR. When the user faces away from the virtual workbench, it may entirely disappear. By spatially fixing the virtual workbench in a fixed but movable location in the OR, it is also easy for the user to locate it, for example, the user merely has to stand in front or facing the direction of the virtual workbench and it will be always available.

Further, in some embodiments, all the information stored and/or integrated in the AR system is available to the user at the virtual workbench. In certain instances, the user may wish to export certain information such that it follows the user even when the user leaves the real area designated for the virtual workbench, for example, the user wishes to have virtual guidance during drilling a screw hole and they export the guidance for drilling of a particular screw hole to their OHMD, leaving behind the rest of the information at the virtual workbench, as described herein. Thereby, further decluttering the view of the user and only displaying the exported elements selected by the user.

The virtual workbench may be used by a user, as shown in FIG. 12A. The virtual workbench may be used by one or more users at the same time. They may look at the same virtual workbench or a different instance of it, e.g., where different information is shown to different users based on the task they are performing. The position, orientation and scale of the virtual elements may or may not be synchronized between users, or they may be adapted to each viewers' individual position, e.g., to each have an optimal view of the same virtual features. The users may be in the same physical location or they may use the same virtual workbench in a different location, e.g., to provide remote assistance. The connection between devices used by different users may be made through a reference in space, through a local, peer-to-peer or internet network.

Certain embodiments comprise methods of accessing the scanning-device and image-storage module 105 at the virtual workbench. The user may access medical images or plans or inventory of medical devices or a combination thereof at the virtual workbench for viewing or planning or guidance purposes. For example, during a surgery, the user may wish to consult the medical images stored in the scanning-device and image-storage module 105 for verification of a surgical step. At the virtual workbench, the user may access the medical images, and then return to surgery immediately. Once the user faces away from the virtual workbench, the user will no longer be able to see the medical images, thereby immediately clearing his view of any augmented elements without much effort. Further, as the interface of the virtual workbench is customizable, individual user profile may be easily created and stored as part of the scanning-device and image-storage module 105 and be retrieved at the virtual workbench.

Patient data, including a list of anatomical landmarks is stored in the scanning-device and image-storage module 105 and is easily retrievable at the virtual workbench. For example, based on a particular surgery, the virtual workbench will prompt the user with potential anatomical landmarks that may be used for a surgical procedure. During the surgery, the user may refer to this list of anatomical landmarks. Additionally, as the user using one or more of the indicated anatomical landmarks during the surgery, it is registered to the common coordinate system via the registration module 114 and it is highlighted at the virtual workbench. This way, the user has an overview of the anatomical landmarks that he has decided to use for a surgical procedure and can easily refer to this information.

Certain embodiments comprise methods of accessing the virtual 3-D model creation module 106 at the virtual workbench. The virtual 3-D creation module 106 is accessible at the virtual workbench. The user may access virtual anatomical models, virtual medical device models, etc stored in the virtual 3-D creation module 106 at the virtual workbench. For example, the user may wish to verify a medical device such as plate against a virtual anatomical model. To do so, standing, facing the virtual workbench, the user may select the virtual anatomical model of an anatomical part, the virtual anatomical model is displayed at the virtual workbench and the user may then verify the plate against the virtual anatomical model. Virtual anatomical model may confirm the verification by displaying a signal such as “a good match or fit” or warn the user if the plate does not match the virtual anatomical model and prompt the user to select another plate or modify the existing plate. Additionally, the virtual 3-D models may be displayed at the virtual workbench and updated in real-time. Alternatively, or additionally, the user may wish to 3-D print the virtual anatomical models or parts thereof for further guidance during a surgical procedure (such as anatomical model of the planned post-op position during an orthognathic procedure) by sending the data to access the additive-manufacturing device (3-D printer) via the control module 124 at the virtual workbench. The virtual 3-D model may relate to anatomical parts, pre-op plan, post-op planned positions, instruments, medical devices, etc.

Certain embodiments comprise methods of accessing the planning module 108 at the virtual workbench. During surgery, a surgeon may wish to access the plan 210 at the virtual workbench. Preferably, intra-op planning is done at the virtual workbench. This may be performed on virtual anatomical 3-D model that are readily available at the virtual workbench.

Certain embodiments comprise methods of accessing the visualization module 110 at the virtual workbench. The surgeon 224, his staff 226 and/or remote participants 228 may access the visualization module 110 at the virtual workbench for guiding/assisting the surgeon during the procedure.

Certain embodiments comprise of methods of accessing the calibration module 112 at the virtual workbench.

Certain embodiments comprise of methods of accessing the registration module 114 at the virtual workbench. For example, one or more markers may be attached to any of the independently moving objects in the actual scene 230 such as the patient. One or more markers may also be attached to objects such an anatomical 3-D patient-specific model which is accessible at the virtual workbench. As both sets of markers are registered to each other and in the same AR system, it is possible to track them at the virtual workbench. This is particularly useful when the surgeon wants to verify certain steps before performing them on the patient, for example, verify the harvested graft size against the implant that will be eventually used along with the graft in the patient.

Objects registered and tracked by the registration module 114 may also be retrieved at the virtual workbench. For example, at the virtual workbench, a 3-D model of the patient may be created intra-op by the virtual 3-D creation module 106 using intra-op information. A generic virtual 3-D model of an implant may be superimposed on the virtual anatomical model of the patient for planning and verifying purposes. Here, at the virtual workbench, the user may also be able to adjust implant using virtual guidance provided. For example, an implant may need to be reshaped to fit the patient. At the virtual workbench, the user may first virtually shape the implant to fit the virtual anatomical model of the patient. Once the fit is confirmed by the user, the user may then proceed to shape the physical implant. Here, using the guidance on and off mode, the user may shape the physical implant using virtual cues provided at the virtual workbench.

Certain embodiments comprise of methods of accessing the control module 124 at the virtual workbench. In certain embodiments, devices (splints, glasses, earplugs, etc) or parts thereof may be manufactured by accessing the control module 124 of the AR system. For example, the surgeon needs a specific attachment for a generic splint to make it patient matched. In this case, the surgeon may access the draw function of the virtual workbench to design the attachment. Once designed, the surgeon or any other user may send the design to the control module 124. The control module 124 receives the design of the attachment and sends it to the additive-manufacturing device integrated in the AR system, and the additive manufacturing devices begins printing the attachment. Once printing is complete, it notifies the surgeon via the virtual workbench icon. As the attachment is printed in a sterile environment, it may be ready for use. The surgeon may now verify the printed part against the generic splint at the virtual workbench to confirm the fit. Once verified, the surgeon may now use the attachment along with the splint during the surgery.

In certain aspects, the surgeon may prefer to bend a standard plate against an anatomical 3-D patient-specific model at the virtual workbench to make the verification step smoother. The virtual workbench provides the surgeon with a dedicated space wherein he may also be able to verify the bent of the plate against a physical anatomical model, if needed.

In certain aspects, by accessing the inventory available at the virtual workbench, the user may perform incremental registration by selecting from the available list an anatomical part, a marker, a marker on an instrument, etc. This way, the system automatically recognizes the registered objects during the remainder of the procedure without the user having to touch each object individually and physically for registration. The user may prefer to perform this step at the start of a surgery.

### Platform (or Interface) of the Virtual Workbench

The virtual workbench comprises of a platform that enables user interaction. The platform allows the user to access all the modules of the AR system at the location of the virtual workbench. The platform is available to all users who may have access to the AR system. The modules of the AR system are described herein.

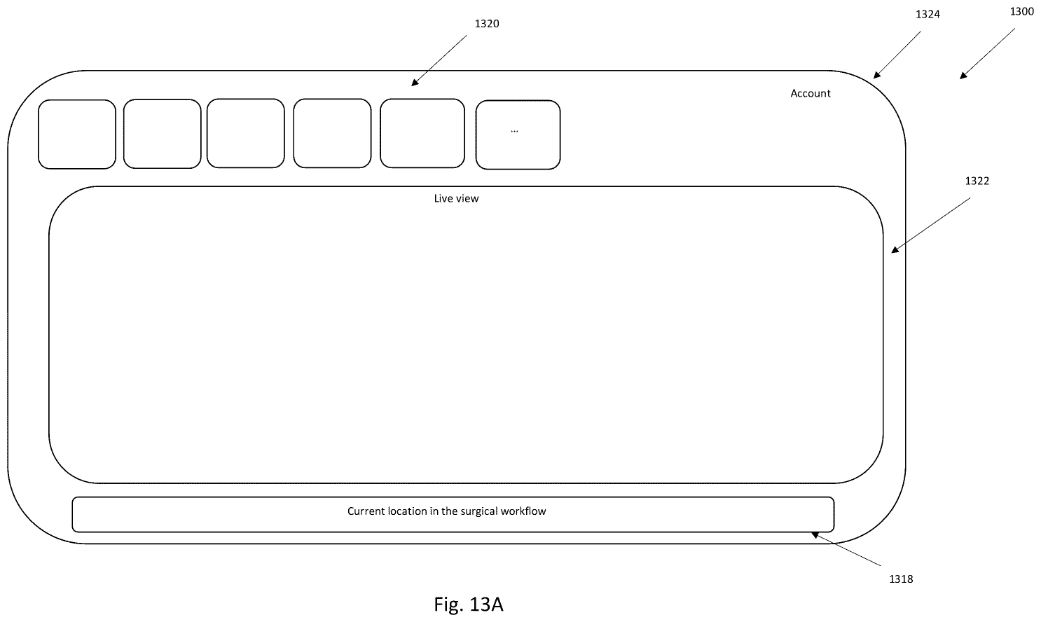

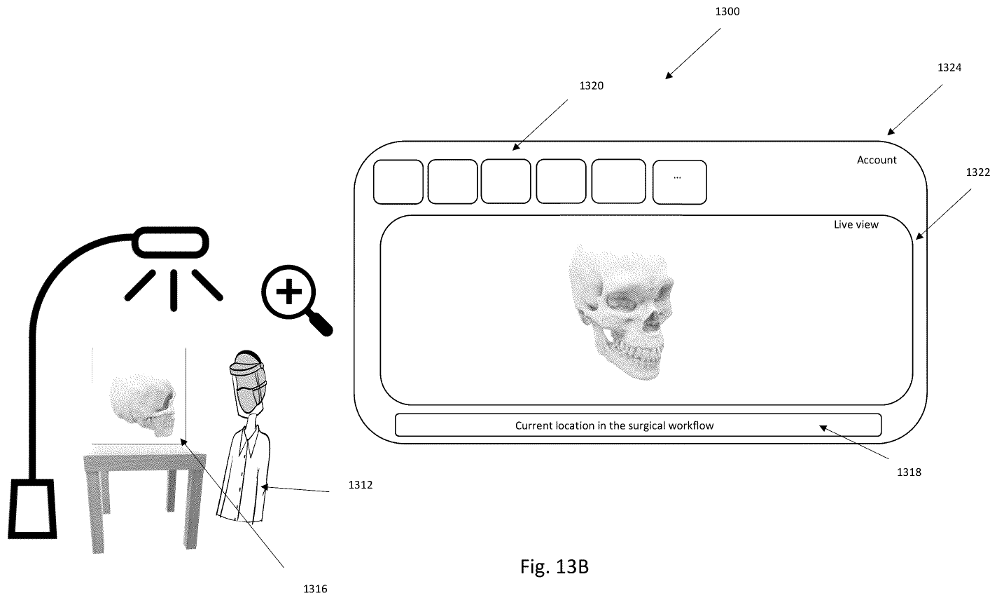

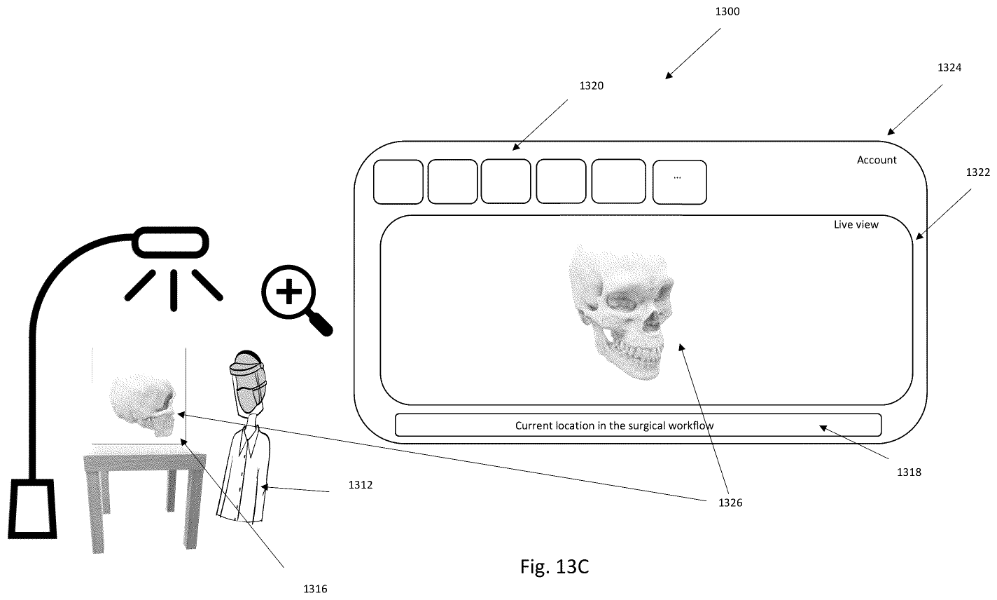

According to certain embodiments, the platform of a virtual workbench is a graphical user interface (GUI) 1300 for use by a user for providing virtual guidance/assistance via an OHMD device (not shown) as shown as FIG. 13A. GUI 1300 provides virtual 2-D and/or 3-D guidance/assistance to a user via AR glasses (OHMD device). Once the user generates a virtual workbench in an OR, a splash screen is visible in his glasses. FIG. 13A illustrates an example of a simple splash screen of a virtual workbench.

Example embodiments, as shown in FIGS. 13A-13C illustrate the user's view when interacting with the virtual workbench and are described herein. The AR system allows the user to access any of its stored and/or real time data via the platform of the virtual workbench.

Through the plurality of tabs, different AR modules may be accessed. An example table below shows the interactions and relations between different tabs and modules of the AR system.

| Menu | | | Scanning | | Virtual | | | | | | | |
|---|---|---|---|---|---|---|---|---|---|---|---|---|
| of | | | device & | | 3D | | | | | | | |
| VWB/ | | Display | image | Case | model | | | | | | | Recording/ |
| Module | I/O | unit | storage | management | creation | Planning | Visualisation | Calibration | Registration | Guidance | Control | streaming |
| View | x | x | x | x | x | x | x | x | x | x | x | x |
| Edit | x | | x | x | x | x | x | x | x | x | | |
| Print | | | x | x | x | x | | | | x | x | |
| Control | x | | | | | x | | | | x | x | x |

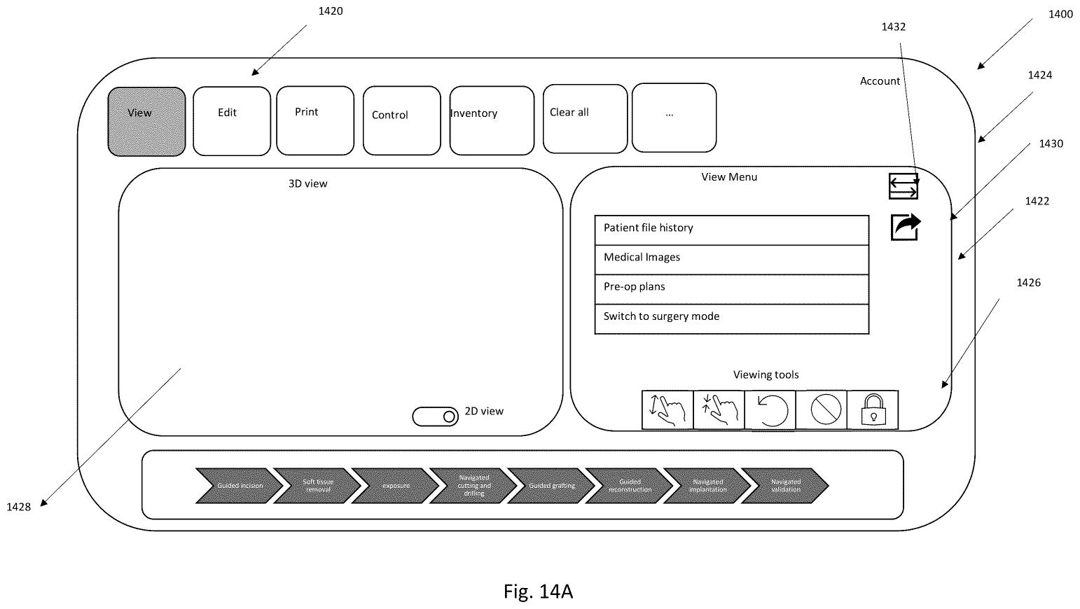

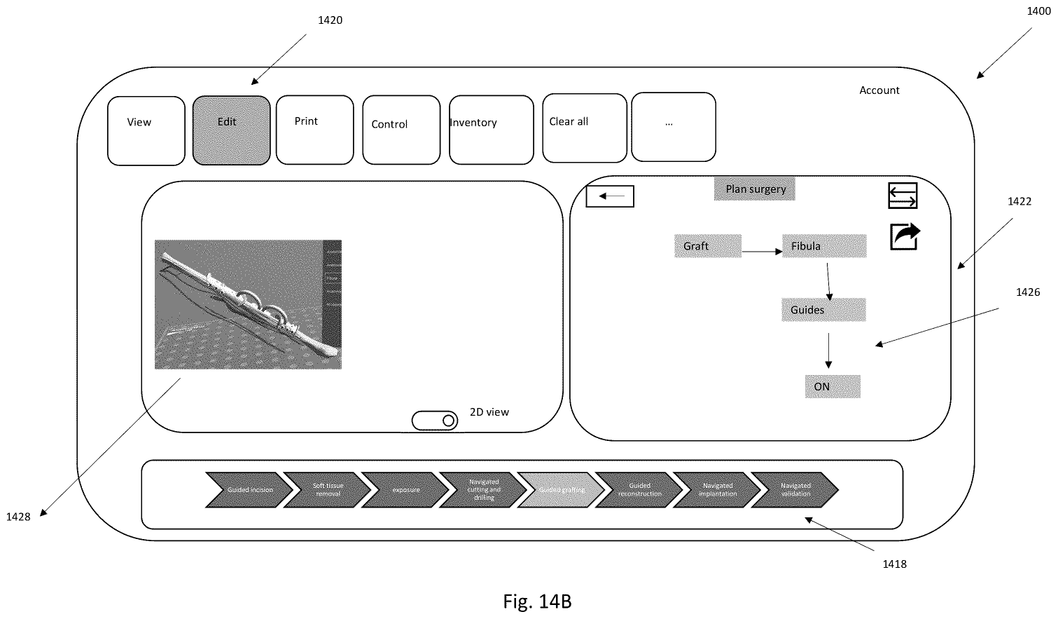

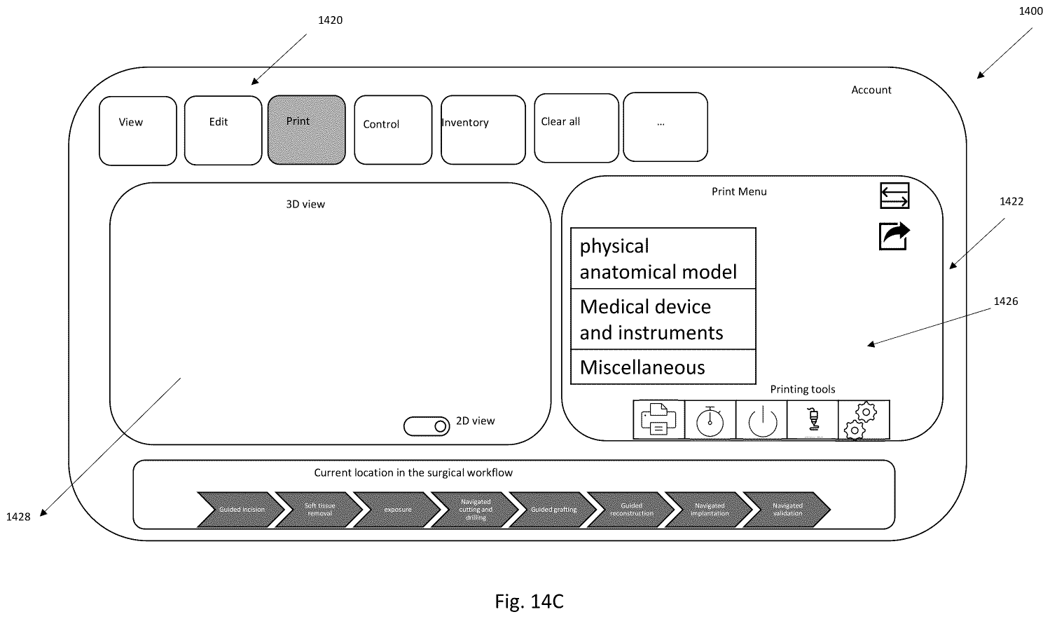

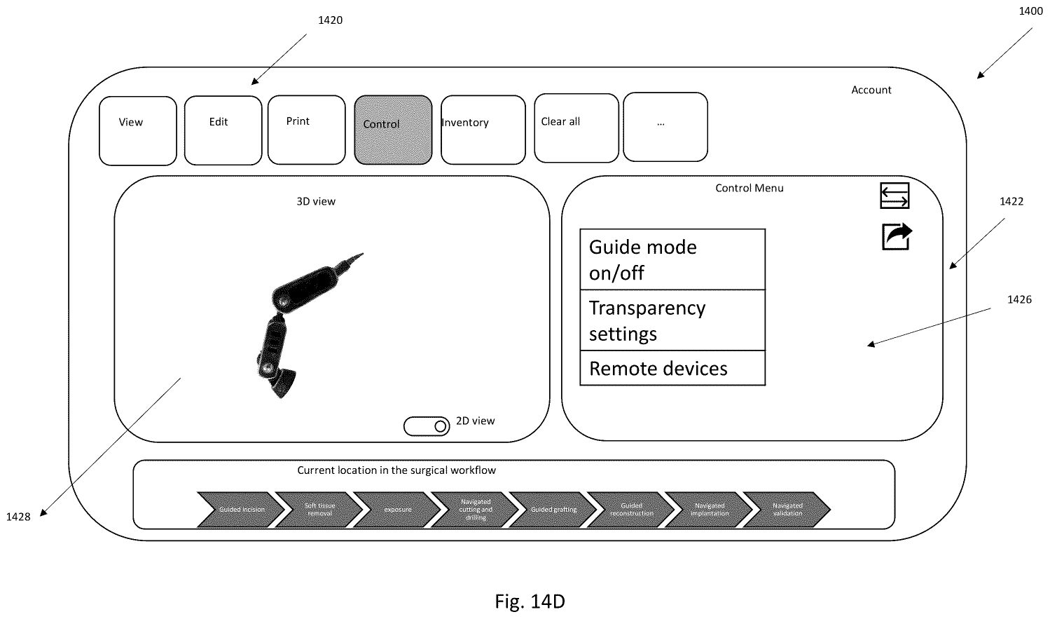

FIGS. 14A-14D, illustrate an example embodiment showcasing a simple GUI of the virtual workbench, and described herein.

The virtual workbench is accessible via the display device module 104 and may be accessible to multiple users at once. They may look at the same virtual workbench or a different instance of it, e.g., where different information is shown to different users based on the task they are performing. The position, orientation and scale of the virtual elements may or may not be synchronized between users, or they may be adapted to each viewers' individual position, e.g., to each have an optimal view of the same virtual features. The users may be in the same physical location or they may use the same virtual workbench in a different location, e.g., to provide remote assistance. The connection between devices used by different users may be made through a reference in space, through a local, peer-to-peer or internet network.

The AR system also automatically updates and works in the background while the surgery is ongoing, without interfering with the ongoing surgery. Additionally, the virtual workbench gives the user the option to export/transfer relevant information that will then be uploaded to the OHMD whilst the remainder is left behind at the virtual workbench. For example, the user may access the pre-op plan at the virtual workbench and decide to export guidance only for a specific step of the surgery (for example, guidance for performing osteotomy), thereby leaving behind the rest of the pre-op plan at the virtual workbench.

The virtual workbench bench and its functionalities may be called upon using any of the embedded AR system options such as voice, gestures, etc. via the I/O module 122 as described herein.

### Example Appearance and Functions of the Virtual Workbench (VWB)

In certain aspects, a VWB provides an interface to the AR system for retrieving, visualizing, guiding, assisting, designing, planning and/or interfacing with other systems (such as robotic arms, 3-D printers, laparoscopes, etc.).

A VWB may provide the user a central place to access (e.g., patient) data available to the AR system, such as medical images, anatomical models, surgical plans, guides, instruments, implants, templates, etc.

A VWB may provide the user access to tools available in the AR system, such as measuring, drawing, annotating, editing, cutting, etc.

A VWB may be configured differently to the user depending on the task that is being performed.

A VWB may be represented in any 2-D or 3-D geometric form, including for example a square, cube, rectangle, cuboid, circle, sphere, triangle, pyramid, or others.

A VWB may be accessible to multiple users of the AR system at the same time.

A VWB may be fixed to one or more locations in physical space. The AR system may use known systems in the art such as SLAM, objects or QR codes, or others, to maintain this fixed position as a user moves in the real world. The user may manipulate the location of a VWB, for example through gestures or voice commands. In certain aspects, when the location of the VWB is determined by the location of a single object in the real world, the VWB will maintain its relative position to that object if this is moved. For example, in a surgery room, SLAM tracking may perform poorly as the layout of the surgery room is often reconfigured. In such cases, the position of the VWB could be fixed to the location of a single surgical table (using object tracking or a reference marker attached to the table), allowing the surgeon to physically reconfigure his operating room (theatre) without losing track of his virtual workbench. In certain aspects, the VWB would thus behave exactly like a physical workbench placed on that table. The AR system can recognize a table surface as well and automatically align the VWB with the table.

A VWB may be hidden or minimized to an icon when it is not being used, for example, through a user action or automatically after a pre-defined time. Such time may be fixed or may depend on the surgical phase or active task. A user action may trigger a VWB to be maximized again. For example, a user may use the VWB for visualizing a presurgical plan in the first phase of the surgery. When he hasn't interacted with the VWB for some time, the system could minimize the VWB to a small VWB icon so that it doesn't disturb the surgery anymore. Afterwards, the user can reactivate the VWB by a gesture or voice command or by relying on eye or gaze tracking when he is focusing on the VWB icon.

A VWB may allow a user to grab any virtual dataset/object and use these during surgery. After use, the virtual data may be returned to the VWB for maintaining a clean workspace. For example, a user may take a planned anatomical model from the VWB to compare it visually to the anatomy on the patient. He may place the anatomical model back in the VWB when he no longer needs it during surgery to avoid decluttering his surgical view. This improves the case-of-use compared to existing AR systems where the virtual space would interfere with the surgical field.

A VWB can be calibrated to ensure that it visualizes data on a scale which is accurate compared to the real size. For example, a physical reference object such as a 2-D marker system or object with known dimensions could be aligned with its virtual counterpart using any calibration method known in the art. The VWB can then visualize all objects such as anatomical models or implants on real-life scale to the user. This is different from existing systems which may use an inaccurate method such as SLAM to determine the scale at which a virtual object is shown on the display in the AR system, which may lead to inaccuracies, for example during the measurement of anatomical features. With a true-to-scale VWB, the surgeon can realistically compare or measure physical objects with virtual ones at the VWB.

An AR system can include of one or more VWB. Each VWB can have a different location in space. Each VWB may be configured for a different task based on their location in the surgery room. The one or more VWB can share the same data in the AR system or may work on different instances of that data. Sharing of data may be achieved as the AR system is using a network connection to transfer data between the AR system and the one or more VWB. For example, a surgery room may have two VWB, where one VWB is located at a sterile table and may be configured for a user to assemble graft components into a construct to be implanted. Another VWB may be located next to the patient and may be configured for another user to navigate the placement of this assembly on to the patient. If changes to the assembly are made in one VWB, the data may be automatically synchronized with the other VWB. This may be done by using a shared database on a network which is part of the AR system and which is accessed using the VWB.

Multiple VWB may not all reside in a single operating room (theatre). For example, in the case of remote surgical assistance, one VWB may be located in the OR with the surgeon and one VWB connecting to the same AR system may be located in the office of a clinical engineer. The clinical engineer can communicate to the surgeon using a recording/streaming module 126 in the AR system. The clinical engineer can then annotate certain features of the anatomy or surgical plan during surgery. The surgeon would see those annotations reflected at the VWB in the OR and can use them during surgery. Alternatively, the clinical engineer can also prepare certain data at the VWB based on a surgeon's instructions, e.g., to make available a specific anatomical model or implant for use during surgery. The surgeon can then conveniently access the prepared data at his own VWB. This would be similar to how a nurse is requested to prepare certain instruments, only for virtual data.

In some cases, the VWB may be used in scenarios where a surgery is performed in different operating rooms (theatres). In such cases, the VWB may serve as the virtual communication tool between the operating room (theatre). In one such example, the AR system and VWB are used for a surgery where donor tissue is used, e.g., in the case of a face transplant. One surgeon may be working on the donor and perform a measurement using the VWB. This measurement can then be made available at the VWB of the surgeon working on the receiver of the donor tissue.

An AR system can allow to pre-configure the layout of the one or more VWB in relation to the physical surgical room, for example based on a virtual model of the surgery room or based on a saved session from a previous use of the AR system. Different to existing AR systems, this would allow a user to immediately start working with the AR system based on the VWB setup, without requiring additional preparation of the virtual part of the surgery.

### An AR System According to Certain Embodiments

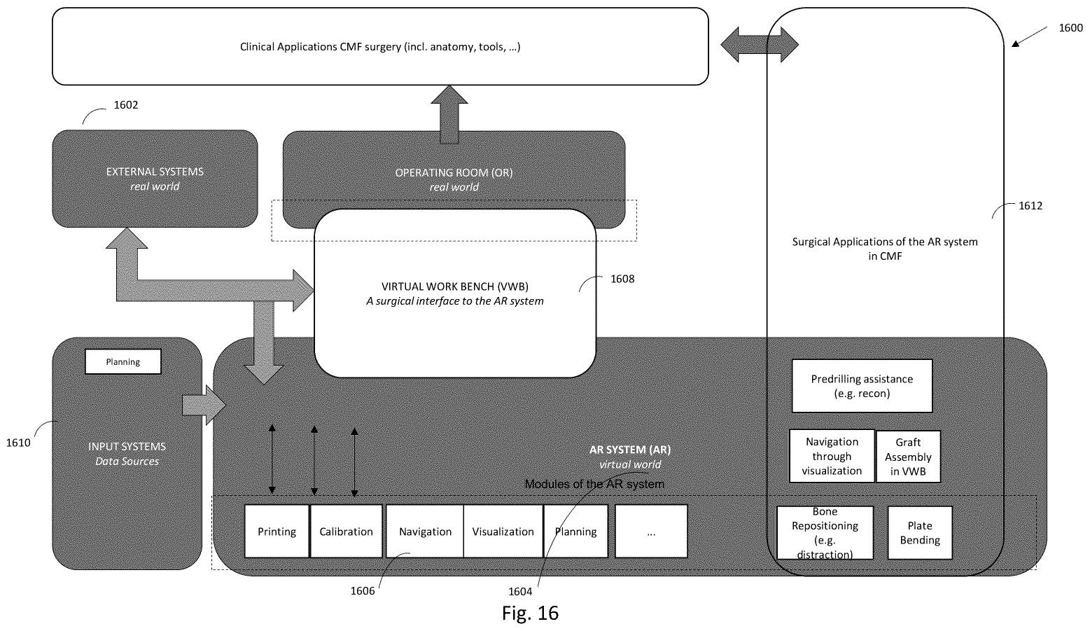

According to certain embodiments, a setup of an AR system in an OR comprising of one or more modules and its interaction with the physical (real) world is shown in FIG. 16. Referring to FIG. 16, the different elements that make up the real world comprise of external systems 1602 (such as imaging devices, additive manufacturing devices, robotics, etc). The virtual world comprises of the AR system 1604, its modules 1606 and the augmented environment. In certain aspects, the virtual workbench 1608 is central to the AR system. It provides the surgical interface to the AR system, from where all the modules of the AR system can be accessed. It also allows the user to operate as a control unit for operating external systems 1602. It may be designed to cater to craniomaxillofacial surgeries. One or more input systems 1610 as described above may feed patient data to one or more modules of the AR system (such as the scanning-device and image-storage module). Further, as described earlier, the external systems 1602 may also be operated in the augmented world via the virtual workbench 1608. Additionally, and similar to the case management module 120, data can be stored in accordance to surgical applications 1612 in the scanning-device and image-storage module 105 of the AR system. For example, specific surgical applications and their methods such as predrilling guidance during a reconstruction surgery, navigation through the visualization module, graft assembly using the virtual workbench, bone repositioning, plate bending, etc. may be stored as separate surgical applications for ease of access. Further, they may be linked to user profile.

### System Requirements

Various embodiments disclosed herein provide for the use of computer software being executed on a computing device. A skilled artisan will readily appreciate that these embodiments may be implemented using numerous different types of computing devices, including both general-purpose and/or special-purpose computing system environments or configurations. Examples of well-known computing systems, environments, and/or configurations that may be suitable for use in connection with the embodiments set forth above may include, but are not limited to, personal computers, server computers, hand-held or laptop devices, multiprocessor systems, microprocessor-based systems, programmable consumer electronics, network PCs, minicomputers, mainframe computers, distributed computing environments that include any of the above systems or devices, and the like. These devices may include stored instructions, which, when executed by a microprocessor in the computing device, cause the computer device to perform specified actions to carry out the instructions. As used herein, instructions refer to computer-implemented steps for processing information in the system. Instructions can be implemented in software, firmware or hardware and include any type of programmed step undertaken by components of the system. Various wearable and/or portable devices for viewing the augmenting environment such as Microsoft Hololens, etc. may be used. These devices may be connected to the computing device wirelessly or wired.

A microprocessor may be any conventional general-purpose single- or multi-chip microprocessor such as a Pentium® processor, a Pentium® Pro processor, a 8051 processor, a MIPS® processor, a Power PC® processor, or an Alpha® processor. In addition, the microprocessor may be any conventional special purpose microprocessor such as a digital signal processor or a graphics processor. The microprocessor typically has conventional address lines, conventional data lines, and one or more conventional control lines.

Aspects and embodiments of the disclosure described herein may be implemented as a method, apparatus or article of manufacture using standard programming or engineering techniques to produce software, firmware, hardware, or any combination thereof. The term ““article of manufacture”” as used herein refers to code or logic implemented in hardware or non-transitory computer readable media such as optical storage devices, and volatile or non-volatile memory devices or transitory computer readable media such as signals, carrier waves, etc. Such hardware may include, but is not limited to, field programmable gate arrays (FPGAs), application-specific integrated circuits (ASICs), complex programmable logic devices (CPLDs), programmable logic arrays (PLAs), microprocessors, or other similar processing devices.

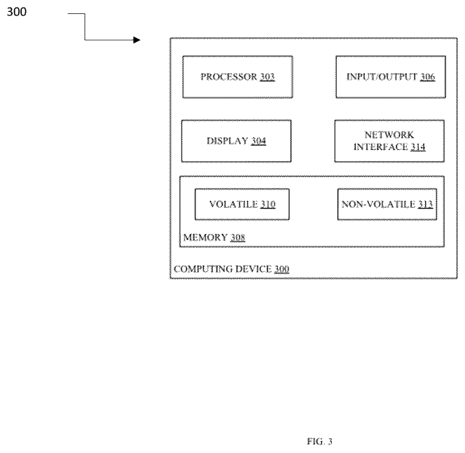

Various embodiments described herein may be implemented using general and/or special purpose computing devices. Turning now to FIG. 3, an example of a computing device 300 suitable for implementing various embodiments described herein is shown. The computer system 300 may generally take the form of computer hardware configured to execute certain processes and instructions in accordance with various aspects of one or more embodiments described herein. The computer hardware may be a single computer, or it may be multiple computers configured to work together. The computing device 300 includes a processor 303. The processor 303 may be one or more standard personal computer processor such as those designed and/or distributed by Intel, Advanced Micro Devices, Apple, or ARM. The processor 303 may also be a more specialized processor designed specifically for image processing and/or analysis. The computing device 300 may also include a display 304. The display 304 may be a standard computer monitor such as, an LCD monitor, an overhead display, and/or a head mounted display, etc. The display 304 may also take the form of a display integrated into the body of the computing device, for example as with an all-in-one computing device or a tablet computer.

The computing device 300 may also include input/output devices 306. These may include standard peripherals such as keyboards, mice, printers, stylus, cameras, sensors, and other basic I/O software and hardware. The computing device 300 may further include memory 308. The memory 308 may take various forms. For example, the memory 308 may include volatile memory 310. The volatile memory 310 may be some form of random-access memory, and may be generally configured to load executable software modules into memory so that the software modules may be executed by the processor 303 in a manner well known in the art. The software modules may be stored in a nonvolatile memory 313. The non-volatile memory 313 may take the form of a hard disk drive, a flash memory, a solid-state hard drive or some other form of non-volatile memory. The non-volatile memory 313 may also be used to store non-executable data, such database files and the like.

The computer device 300 also may include a network interface 314. The network interface may take the form of a network interface card and its corresponding software drivers and/or firmware configured to provide the system 300 with access to a network (such as the Internet, for example). The network interface card 314 may be configured to access various different types of networks, such as those described above in connection with FIG. 1. For example, the network interface card 314 may be configured to access private networks that are not publicly accessible. The network interface card 314 may also be configured to access wireless networks such using wireless data transfer technologies such as EVDO, WiMax, or LTE network. Although a single network interface 314 is shown in FIG. 3, multiple network interface cards 314 may be present in order to access different types of networks. In addition, a single network interface card 314 may be configured to allow access to multiple different types of networks.

In general, the computing environment 100 shown in FIG. 1 may generally include one, a few, or many different types of computing devices 300 which work together to carry out various embodiments described herein. A skilled artisan will readily appreciate that various different types of computing devices and network configurations may be implemented to carry out the inventive systems and methods disclosed herein.

### Preparation of Material for Surgery

Aspects of the present disclosure relate to systems and methods for operating devices using the AR system during a surgical procedure.

Aspects of the present disclosure relate to using one or more modules of the AR system for operating medical devices and/or medical instruments during a surgical procedure.

Aspects of the present disclosure relate to using one or more modules of the AR system for operating medical devices and/or medical instruments during a craniomaxillofacial surgery.

Aspects of the present disclosure relate to systems and methods for providing guidance/assistance in preparing surgical material that may be used during execution of one or more surgical steps such as preparation of donor graft, resection of tumor, adapting standard medical devices (implants) to fit patient(s).

Aspects of the present disclosure relate to systems and methods for registering and tracking medical devices such as implants, surgical guides, etc by using the AR system for live guidance/assistance.

Aspects of the present disclose relate to systems and methods for registering and tracking medical instruments such as drill bits, screws, sawblades, etc. by using the AR system for live guidance/assistance.

One or more modules of the AR system may be used for operating a plurality of medical devices, medical instruments, anatomical models, etc. as described herein.

In certain embodiments, one or more modules of the AR system may be used for adapting standard medical devices into personalized solutions as described herein.

Medical devices may be implanted in a patient for short or long term. Medical devices may be standardized or personalized or standard but adaptable, depending on the defect/deformity/surgeon preference, etc. Medical devices comprise of one or more of screws, plates, implants, surgical wires, surgical guides, splints, etc. One or more medical devices, standard, personalized or a combination thereof may be used during a surgical procedure as described herein.

Certain embodiments comprise systems and methods of providing personalized assistance/guidance (or solution) such as personalized (or patient matched or customized or patient-specific) medical devices (implants, surgical guides or combination thereof), personalized planning (surgical plan, medical device design, implementation of said surgical plan or combination thereof), or a combination thereof for one or more surgical procedures as described herein.

Certain embodiments comprise devices and systems of providing personalized solutions for a surgical procedure such as medical devices (standard or personalized or a combination of both), medical instruments (standard, personalized or a combination of both), surgical planning or a combination thereof.

Certain embodiments comprise systems, methods and devices of providing personalized solutions for a surgical procedure such as medical devices (standard or personalized or a combination of both) and medical instruments (standard, personalized or a combination of both).

Certain embodiments comprise systems, methods and devices of providing personalized solutions for a surgical procedure such as medical devices (standard or personalized or a combination of both), and surgical planning.

Certain embodiments comprise systems, methods and devices of providing personalized solutions for a surgical procedure such as medical instruments (standard, personalized or a combination of both) and surgical planning or a combination thereof.

Certain embodiments comprise systems and methods of providing personalized solutions for a surgical procedure such as surgical planning or a combination thereof.

Certain embodiments comprise systems and methods of providing guidance for personalizing medical devices. For example, the AR system (and the virtual workbench) is used for reshaping/adapting a standard plate into a patient match plate for a surgical procedure (such as orthognathic surgery). Additionally, or alternatively, the AR system may also be used for creating a surgical plan for a surgical procedure as described herein. Further, the AR system may also provide step-by-step guidance during a surgical procedure as described herein.

Standard medical devices such as implants, plates, screws, surgical wires, etc. are routinely used in medical surgeries. These are manufactured in various shapes and sizes. There are a number of standard devices that can be adapted to fit a patient such as orthognathic plates, etc. that are generally adapted in the OR. Various medical instruments such as plate bending forceps, plate and wire cutting pliers, distractors for S plate, etc. are also part of a surgical kit.

Medical devices that may be customized, personalized, patient-matched or patient-specific are also routinely used in medical surgeries. These are manufactured in accordance with patient features and are made to fit patient-matched bony anatomy of a particular patient.

In certain aspects, standard medical devices may be adapted to personalized medical devices.

The medical devices to be adapted can be made out of several materials, including metal and metal alloys: e.g., commercially pure titanium, tantalum, Ti alloys, Co—Cr alloys, stainless steel, ceramics: e.g.; zirconia, synthetic polymers: e.g., PEEK, polyamide, porous polyethylene biodegradable and bioresorbable materials: e.g., PCL, PLA, natural materials or biological tissues: e.g., bio printed, autologous bone graft, alloplastic bone graft, BMP, Vivigen, or particulate bone.

During a surgical procedure, a surgeon may require a plurality of medical instruments for carrying out a plurality of actions. During a craniomaxillofacial surgery, medical instruments used by a surgeon may comprise of dental splints for tracking the movements of jaw(s) or parts thereof, drill bits for drilling one or more holes in the bone, saw blades for performing osteotomies on the patient, surgical markers for marking/highlighting patient anatomy, screws (self-drilling, self-tapping, locking, emergency, graft) for fixing implant or implant components, implants such as plates (straight, straight double, curved, double curved, straight adjustable, L-shaped, T-shaped, X-shaped, Y-shaped, I-shaped, S-shaped, square, le fort segmented, (100) degree specific, on-site adjustable, condylar high fracture locking, condylar fracture locking, mandibular, mandibular locking, hemimandibular locking, hemimandibular, total mandibular locking, total mandibular, mini, micro, locking, medium, large, thick, thin), screwdrivers for fixing screws, plates with spacing length for measuring distance between two screw holes, wire and plate cutting pliers, screw holding forceps, plate holding forceps, applying instrument, plate bending forceps, screwdriver handle, screwdriver blade, intermaxillary fixation screw blade, long hexagonal mandrel, flat forceps, handle with trocar sleeve, check retractor, drill guide, trocar, plate bending lever, depth gauges for drill-holes, 3 point pliers, templates (hemimandibular, total hemimandibular, full mandibular, angle/ramus, symphysis), instruments (such as sagittal saws, oscillating saws, reciprocating saws, etc.) for tumor resection and graft harvesting. In certain embodiments, for example during complex surgeries, surgeon may also use physical anatomical models for additional guidance/assistance. Physical anatomical models may be designed to represent pre-op anatomy or post-op. Additionally or alternatively, the surgeon may use virtual anatomical models created by the virtual-3-D-model-creation module 106 for planning, assistance or guidance during a surgery.

During a surgical procedure, a surgeon may require a plurality of medical instruments for carrying out various actions to accurately recreate the surgical plan. These can include surgical guides, comprising of polyamide or titanium, corresponding custom plates and/or implants, dental splints, and anatomic bone models. Anatomical models may comprise of physical and virtual models based on pre-op or intra-op or post-op patient anatomy. Physical anatomical models may be manufactured using additive manufacturing technology (3-D printed) or non-3-D printed technology. Depending on user preference, 3-D printed anatomical models may be made of polyamide or clear acrylic. Non-3-D printed anatomical models are generally made of Gypsum (e.g dental plaster cast). Virtual anatomical models may be created using the virtual-3-D-model-creation module 106 as described herein. During a surgical procedure, a combination of physical and virtual models may be used, for example during the plate bending steps of an orthognathic surgery, a surgeon may use the physical anatomical model to verify the bent plate while using virtual model of a plate to guide the process of bending, as described herein. Models can also be used for visual reference to check osteotomy accuracy, verifying the fit and placement of guides and/or plates, and communicating the surgical plan to colleagues or in a teaching environment.

Certain embodiments comprising methods of operating one or more medical devices for a surgical procedure using the AR system are described herein.

Certain embodiments comprising methods of operating one or more medical instruments for a surgical procedure using the AR system are described herein.

Certain embodiments comprising methods of operating one or more medical devices for a craniomaxillofacial surgery using the AR system are described herein.

Certain embodiments comprising methods of operating one or more medical instruments for a craniomaxillofacial using the AR system are described herein.

Certain embodiments comprising methods of operating one or more medical devices using the AR system for an orthognathic surgery are described herein.

Certain embodiments comprising methods of operating one or more medical devices using the AR system for a mandible reconstruction surgery, are described herein.

Certain embodiments comprising methods of operating one or more medical devices using the AR system for a maxilla reconstruction surgery, are described herein.

Certain embodiments comprising methods of operating one or more medical devices using the AR system for a bi jaw (maxilla and mandible) reconstruction surgery, are described herein.

Certain embodiments comprising methods of operating one or more medical devices using the AR system for an orbital-floor reconstruction surgery, are described herein.

Certain embodiments comprising methods of operating one or more medical devices using the AR system for a tumor-resection and reconstruction surgery of one or more anatomical parts of the CMF region, are described herein.

Certain embodiments comprising methods of operating one or more medical devices using the AR system for a trauma surgery of one or more anatomical parts of the CMF region, are described herein.

Certain embodiments comprising methods of operating one or more medical devices using the AR system for a craniosynostosis surgery, are described herein.

Certain embodiments comprising methods of operating one or more medical instruments using the AR system for an orthognathic surgery are described herein.

Certain embodiments comprising methods of operating one or more medical instruments using the AR system for a mandible reconstruction surgery, are described herein.

Certain embodiments comprising methods of operating one or more medical instruments using the AR system for a maxilla reconstruction surgery, are described herein.

Certain embodiments comprising methods of operating one or more medical instruments using the AR system for a bi jaw (maxilla and mandible) reconstruction surgery, are described herein.

Certain embodiments comprising methods of operating one or more medical instruments using the AR system for an orbital-floor reconstruction surgery, are described herein.

Certain embodiments comprising methods of operating one or more medical instruments using the AR system for a tumor-resection and reconstruction surgery of one or more anatomical parts of the CMF region, are described herein.

Certain embodiments comprising methods of operating one or more medical instruments using the AR system for a trauma surgery of one or more anatomical parts of the CMF region, are described herein.

Certain embodiments comprising methods of operating one or more medical instruments using the AR system for a craniosynostosis surgery, are described herein.

### Applications for Using the AR System in an OR

### General

Aspects of the present disclosure relate to systems, methods and devices for providing assistance/guidance during a surgical procedure on a plurality of anatomical parts of a patient.

Aspects of the present disclosure relate to systems, methods and devices for providing (personalized) assistance/guidance during a surgical procedure using plurality of guidance elements (such as guides), plurality of personalized medical devices (such as implants) and/or combination thereof.

According to an embodiment, the systems and methods provide visual guidance/assistance by augmented reality system during craniomaxillofacial (CMF) surgery.

Certain aspects relate to using the I/O module 122 of the AR system during a surgical procedure as described herein.

Certain aspects relate to using the display unit 104 of the AR system during a surgical procedure as described herein.

Certain aspects relate to using the scanning-device and image-storage module 105 for retrieving, storing or modifying patient data as described herein.

Certain aspects relate to using the case management module 120 for retrieving, patient files (or case).

Certain aspects relate to using virtual-3-D-model-creation module 106 of the AR system for creating virtual anatomical and/or virtual medical device models as described herein.

Certain aspects relate to using the planning module 108 of the AR system for accessing, modifying, creating a surgical plan as described herein.

Certain aspects relate to using the visualization module 110 of the AR system for visualizing anatomical landmarks, medical devices, medical instruments or combination thereof as described herein.

Certain aspects relate to using the calibration module 112 of the AR system for calibrating medical devices or instruments or combination thereof as described herein.

Certain aspects relate to using the registration module 114 of the AR system for registering the patient or the medical devices or medical instruments or anatomical models or combination thereof as described herein.

Certain aspects of the present disclosure relate to using the guidance module 116 of the AR system and methods for guiding instruments, for example, for drilling, placing pins, positioning of splints (intermediate, final or palatal), positioning of device or device components such as implants on a bony anatomy during a surgical procedure as described herein.

Certain aspects relate to using the control module 124 of the AR system for operating one or more external systems during a surgical procedure as described herein.

Certain aspects relate to using the recording/streaming module 126 for retrieving, storing or modifying patient data as described herein.

Certain aspects relate to accessing one or more modules of the AR system via the virtual workbench during a surgical procedure as described herein.

Certain aspects of the AR system disclosed herein may be used for guiding external systems such as robotics, provide guidance to users for performing plurality of surgical steps such as cutting bones, removal of bone or bone parts, removal of cartilage, removal of tissue, resection of tissue.

Certain embodiments comprising methods of using one or more modules of the AR system for registering landmarks which leads to better patient outcomes due to its accuracy.

AR guidance may be provided in the form of step-by-step guidance and/or displaying safety/warning signs by the guidance module 116 of the AR system.

AR guidance may also be provided for adapting standard instruments into customized versions such as during the process of plate bending, intra-op guidance during placement of implant and also during post-op.

According to certain embodiments, the systems and methods provide visual guidance/assistance by augmented reality system during craniomaxillofacial (CMF) surgery, such as orthognathic surgery, reconstruction surgery, CMF trauma reconstruction for (e.g.: such as fractures of the zygoma, orbital floor, sinus, skull base, cranial vault, midface, nasal NOE, tooth, alveolar process, mandible, maxilla), CMF oncological reconstruction, orthognathic surgery, CMF distraction, CMF aesthetic reconstruction, craniofacial surgery (e.g., such as craniosynostosis, congenital deformities, etc.).

Although certain aspects that follows describe embodiments of systems and methods being used for craniomaxillofacial surgery, the systems and methods can similarly be used during non-CMF surgical procedures as well such as of pelvic/acetabular fracture, spinal rods, spinal osteosynthesis and fusion plates, modular implant systems (e.g., such as lower extremity mega prosthesis), forearm osteotomy such as (distal radius reconstruction), veterinary osteosynthesis applications, extremity osteosynthesis plates (hand, foot, ankle), external fixators. Parts of the system may also be used in non-skeletal surgeries, e.g., during minimally invasive procedures, pulmonary or cardiac valve interventions.

Various clinical indications may lead to craniomaxillofacial surgery (CMF). For example, the aims of surgery can include removal of malignant cells, restoring function, aesthetics and/or eliminating pain in the craniomaxillofacial region. Several surgical procedures are used, depending on the clinical indication. For example, orthognathic surgery will correct for functional or aesthetic limitations caused by malalignment of the jaw. Reconstructive surgery may be used to remove a tumor and reconstruct the anatomy to a normal state. Trauma surgery may be used to treat pain, functional loss or aesthetic problems after fractures. Surgery often involves the use of implants and/or implant components as part of treatment. The correct positioning of these implant components in relation to the bony anatomy (e.g., mandible, maxilla, orbital floor, cranium) may be crucial in achieving good patient outcome.

Some surgical interventions are intended to correct bone deformations, occurrences of disharmony or proportional defects of the body, in particular, the face, or post-traumatic after-effects. These interventions use actions for repositioning, in an ideal location, some fragments of bone which have been separated from a base portion beforehand by a medical professional. Some surgical interventions are intended towards restoring bone defects. The restoration process may be completed by means of one or more bone grafts, bone substitutes, personalized medical devices (implants) or a combination thereof.

In certain aspects, surgical interventions therefore comprise an osteotomy which is carried out in order to release one or more badly positioned bone segments; for example, to move this or these bone segment(s), that is to say, to move it/them by way of translation and/or by rotation in order to be able to reposition it/them at their ideal location. In case of trauma or reconstruction surgeries, the surgical intervention may also involve the use of one or more bone grafts. Further, osteotomies may be performed to remove segments of the native bone that are not repositioned but removed in any case e.g., bone segment that has a tumor growth. A tumor growth may be benign or malignant.

In certain aspects, when all bone segments occupy a new position, the surgeon fixes the bone segments to other adjacent bone portions of the patient using one or more implants.

In certain CMF surgeries, along with using medical devices, grafts may also be used, such as in reconstructive surgeries. The grafts may be one or more of autografts, allografts, generic grafts, isografts, xenografts, cadaveric, vascularized (free-flap), non-vascularized grafts or a combination thereof. These grafts are harvested from one or more of donor sites such as scapula (CSA), hip (iliac crest/DCIAS), calvarial, radius, rib, knee (femoral condyle), fibular free flap (vascularized with 1 artery and 2 veins), femur, or soft tissue donor sites.

Certain embodiments comprising methods of harvesting, reshaping and implanting one or more grafts using one or more modules of the AR system are described herein.

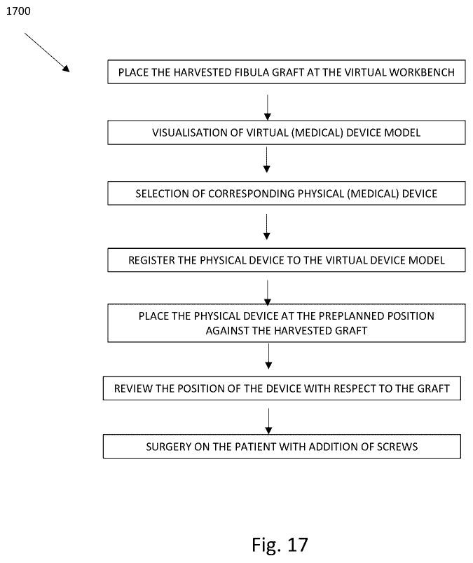

Certain embodiments comprising methods of reshaping one or more grafts using the virtual workbench is illustrated in FIG. 17.

Amongst the various forms of surgery which affect the facial skeleton, it is possible to mention:
orthognathic surgery, the objective of which is to reposition dentition in relative comfortable positions, ensuring good engagement of the teeth; such an intervention involves a maxillary osteotomy if it is necessary to move the upper dental bridge, or a mandibular osteotomy if it is necessary to move the lower dental bridge, or a bi-maxillary osteotomy if it is advantageous to move segments of bone on the two jaws in order to also re-establish the normal proportions of a face,
genioplasty involving an operation on the chin of a patient for aesthetic matters (in order to correct a protruding chin or in contrast a receding chin) or for functional matters, for example, allowing a patient to be able to move his lips into contact with each other without effort,
the correction of post-traumatic after-effects, for example, with regard to an anatomical structure in the face such as zygomatic bone or orbital wall, following accidental impacts.

reconstruction of one or more anatomical parts due to trauma, oncology or congenital defects (such as orbital hypertelorism, Treacher Collins syndrome (TCS), cleft lip and palate (CLP), hemifacial microsomia (HFM), fibrous dysplasia). For example, reconstructions of the mandible and/or maxilla are essential for restoring the patient's quality of life, since these anatomies are essential for the masticatory and phonetic functions, support the teeth and define the shape of the lower part of the patient's face. These defects could also impact the breathing of the patient.

Skull reconstructions for defects due to tumor, trauma or infection are another example, involving restoring the bone defect next to muscle, fat and skin defects to establish a more normal structure and appearance of the patient. Skull reconstruction for craniosynostosis (i.e. a birth defect in which the fibrous joints between the bones of baby's skull close before the brain is fully developed) can be required in order to relieve the pressure within the skull, allow for brain growth, and improve the appearance of the shape of the child's head.

Other surgeries of the craniomaxillofacial region may include facial nerve surgery caused due to paralysis (both, reversible and irreversible), bone lengthening surgeries of maxilla and/or mandible, deformities of the sequela such as of the skull base and cranial vault, of the midface, of the mandible, etc.

Patient data plays an important role during diagnosis, virtual planning and execution of a surgical procedure as already described herein. Anatomical landmarks may be used during virtual guidance of a surgery as already described herein. Anatomical landmarks may be registered and tracked using the AR system.

Patient data may be stored in the scanning-device and image-storage module 105 of the AR system for it to be retrieved during a surgical procedure as described herein.

Patient data such as medical images, dental scans, patient history, anatomical landmarks, cephalometric landmarks, etc may be retrieved from the scanning-device and image-storage module 105 for guidance, reference and/or virtual planning purposes during a surgical procedure as described herein.

An illustrative non-exhaustive list of anatomical landmarks of the craniomaxillofacial region is given below. One or more combinations of these landmarks may be used during different surgical interventions as described herein. For example, anatomical landmarks may be used for visualizing and/or registering virtual and live data of a patient.

| | |
|---|---|
| Skull | Neurocranium bones (occipital, sphenoid, squamous, mastoid portion |
| | of the petrous, temporal, parietal, frontal) |
| | Viscerocranium (ethmoid bone, inferior nasal conchae, lachrymal |
| | bones, nasal bones, the vomer, maxilla, incisive bones, palatine bone, |
| | zygomatic bone, tympanic bones, mandible, hyoid) |
| | Sutures - metopic, coronal, sagittal, lambdoid, squasmosal |
| Mandible | Body (alveolar process, alveolar juga, mental protuberance, mental |
| | tubercle, mental foramen, mandibular angle), ramus (anterior coronoid |
| | process, posterior condylar process), mandibular notch, neck of the |
| | condylar process, pterygoid fovea, masseteric tuberosity, mandibular |
| | foramen, lingula of the mandible, mylohyoid groove, pterygoid |
| | tuberosity, mylohyoid line, submandibular fossae, sublingual fovea, |
| | interalveolar septa, mental spine, digastric fossae. |
| Orbital | Orbit, frontal bone, lesser wing of sphenoid bone, zygomatic bone, |
| cavity | greater wing of sphenoid bone, maxilla, palatine, ethmoid bone, |
| | lachrymal bone, superior orbital fissure, inferior orbital fissure, infra |
| | orbital groove, intra orbital canal, infra orbital margin, infra orbital |
| | foramen, zygomatic orbital foramen, ethmoid foramina, lachrymal |
| | crests, nasolacrimal canal |
| Nasal | Nose, nasal septum, piriform aperture, choana, posterior process, |
| cavity | cartilage, perpendicular plate of the ethmoid, sphenoidal crest, vomer, |
| | cribriform plate of the ethmoid, conchae nasales, inferior concha, |
| | sphenoethmoidal recess, sphenoethomoidal foramen, perpendicular |
| | plate of the palatine bone, uncinate process, maxillary hiatus, ethmoidal |
| | bulla, ethmoidal infundibulum. |
| Temporo- | Head of the mandible, mandibular fossa, articular tubercle, external |
| mandibular | acoustic meatus, medial cranial fossa, condylar process, zygomatic |
| joint | arch, glenoid fossa eminence |
| (TMJ) | |
| Soft tissue | Glabella, nasal tip, cupid's bow/philtrum, medial canthus, lateral |
| | canthus, subnasalae, tragus, soft tissue nasion, soft tissue pogonion |

Certain embodiments comprise methods of using one or more anatomical landmarks for visualization during a surgical procedure. As described herein, visualization of anatomical landmarks is executed by the visualization module 110 of the AR system. For example, critical structures such as teeth, parts of teeth, teeth roots, nerves, foramina, lacrimal system, orbital inferior fissure, optic nerve may be visualized as regions to avoid during a surgical procedure such as orthognathic or orbital-floor reconstruction or temporomandibular jaw surgery, etc.

Certain embodiments comprise methods of using one or more anatomical landmarks for registering, tracking, visualizing or guiding planned cuts, burrs, etc. for additional guidance during a surgical procedure. For example, overlay of burred bone, visualization of osteotomy lines, places, tracking of saw blades for performing said osteotomies may also be executed by one or more modules of the AR system such as the visualization module 110, the calibration module 112, the registration module 114, the guidance module 116, etc. One or more modules may interact with each other during the execution, as described herein.

Certain aspects comprise of using one or more anatomical landmarks for registering, tracking, visualizing and/or guiding (freehand) contouring during a surgical procedure such as facial feminization, complex reconstructions involving contouring of one or more grafts, during fibrous dysplasia, etc., may also be executed by one or more modules of the AR system such as the visualization module 110, the calibration module 112, the registration module 114, the guidance module 116, etc. One or more modules may interact with each other during the execution, as described herein.

Certain aspects comprise of using one or more anatomical landmarks for registering, tracking, visualizing and/or guiding volumes of anatomical structures that may be resected, volume margins to be maintained, etc. during a surgical procedure. For example, orbital volume, intracranial volume, even airway/nasal space, visualizing pre-op or intra-op state, visualizing planned and/or symmetrical values, etc. during an orbital-floor reconstruction or a cranial vault reconstruction or a trauma surgery may be executed by one or more modules of the AR system such as the visualization module 110, the calibration module 112, the registration module 114, the guidance module 116, etc. One or more modules may interact with each other during the execution, as described herein

Apart from anatomical landmarks, a surgeon may also use cephalometric analysis during virtual planning. Cephalometric analysis is the analysis of the relationship between the dental and skeletal regions of a human skull. Cephalometric landmarks serve as important points of references during measurement and analysis. Landmark points may be joined by lines to form axes, vector, angles and planes. Illustrative, non-exhaustive list of cephalometric landmarks is given below. One or more combinations of these landmarks may be used during different surgical interventions, as described herein.

point

| | |
|---|---|
| A point | The deepest point of the curvature of the anterior maxilla |
| (subspinale or A) | between the ANS and the alveolar crest. Although the A point ma |
| | change with treatment, it represents the most forward point of the |
| | maxilla |
| Anterior nasal | The most anterior projection of the anterior projection of the ANS |
| spine (ANS) | of the maxilla in the median plane |
| Articulare (Ar) | The point of intersection of the posterior margin of the ascending |
| | ramus and the outer margin of the cranial base |
| B point | The most posterior point on the outer curve of the mandibular |
| (supramentale, | alveolar process between the alveolar crest and the bony chin. The |
| or B) | B point delineates the most anterior point of the mandible in the |
| | median plane |
| Basion (Ba) | The most inferior posterior point on the occipital bone that |
| | corresponds to the anterior margin of the foramen magnum |
| Condylion (Cd) | The most superior point on the articular head of the condyle |
| CF point (center | The cephalometric landmark formed by the intersection of the |
| of face) | Frankfort horizontal plane and a perpendicular line through point |
| Dental | Most prominent point on the cusp of maxillary canines, |
| Landmarks | mandibular canines, mesiobuccal cusp of maxillary molars, |
| | mesiobuccal cusp of mandibular molars, point centered between |
| | maxillary central incisors, point centered between mandibular |
| | central incisors |
| Gnathion (Gn) | A constructed point that is formed by the intersection of the facial |
| | and mandibular planes |
| Gonion (Go) | Another constructed point that is represented by the intersection |
| | of the lines tangent to the posterior margin of the ascending ramus |
| | and the mandibular plane |
| Menton (Me) | The most inferior point of the mandibular symphysis |
| Nasion (N) | The external junction of the nasofrontal suture in the median |
| | plane. If the suture is not visible, this point is located at the |
| | deepest concavity of the two bones |
| Orbitale (O) | The most inferior point on the external border of the orbit |
| Pogonion (Pg) | The most anterior point on the midsagittal mandibular symphysis |
| Posterior nasal | The tip of the posterior spine of the palatine bone. This landmark |
| spine (PNS) | is usually not visible even on well exposed lateral heads films; |
| | therefore it is a constructed point that is represented by the |
| | intersection of a continuation of the anterior wall of the |
| | pterygopalatine fossa and the floor of the nose. It also denotes the |
| | posterior limit of the maxilla |
| Porion (Po) | A point located at the most superior point of the external auditory |
| | meatus or the superior aspects of the metal ring that is a |
| | component of the left ear rod of the cephalostat |
| Pt point (Pt) | The intersection of the inferior border of the foramen rotundum |
| | with the posterior wall of the Ptm |
| Pterygomaxillary | A teardrop- shaped fissure, the posterior wall of which is created |
| fissure (Ptm) | by the anterior borders of the pterygoid plates of the sphenoid |
| | bone, the anterior wall represents the posterior border of the |
| | maxilla (maxillary tuberosity). The tip of this fissure denotes the |
| | posterior extent of the maxilla. |
| Sella Turcica (S) | The center of the hypophyseal fossa |

Certain aspects comprise methods of using cephalometric landmarks for guiding a surgeon during a surgery by overlaying (or visualizing) points, planes, angles, vectors, etc., during a surgical procedure.

Cephalometric landmarks are stored in the scanning-device and image-storage module 105 of the AR system for easy retrieval during a surgical procedure. One or more cephalometric landmarks may be used during a surgical procedure.

### Orthognathic

In certain embodiments, the AR system is used during an orthognathic procedure. A typical process of an orthognathic surgery is described herein. It is to be understood that a surgeon may deviate from one or more steps depending on the nature of the surgical procedure.

Orthognathic surgery, also known as corrective jaw surgery, is aimed at correcting conditions of the jaw and lower face. Deformities can be categorized into sagittal, transverse hyperplasia of maxilla, transverse hypoplasia of maxilla, vertical maxilla hyperplasia, vertical maxilla hypoplasia. The sagittal deformities are sub-categorised into maxillary prognathism, maxillary retrognathism, maxillary alveolar protrusion, maxillary alveolar retrusion. Orthognathic surgery may be single jaw surgery performed on either the mandible or maxilla or may be a two-jaw surgery involving both the mandible and the maxilla of a patient. Based on the diagnosis, deformity and jaw involved, an osteotomy may be planned. An osteotomy to be performed on the maxilla may be a LeFort I, high LeFort I, LeFort II, or LeFort III. Additionally, the maxilla can be split into multiple pieces to address severe transverse or other alignment discrepancies. Similarly, an osteotomy to be performed on the mandible may be a bilateral split osteotomy (BSSO), a vertical ramus osteotomy (VSO), an inverted L osteotomy, a subapical osteotomy, and/or a genioplasty (chin correction). In a two-jaw surgery, one or more types of mentioned osteotomies may be combined. Apart from planning of an osteotomy, an important metric taken into account is the occlusion of the jaws. The occlusion may initially be classified into class I, II or III, with the aim of restoring normative class I occlusion through surgical means, orthodontics, or a combination.

Certain embodiments comprise of systems and methods of using the AR system during an orthognathic surgery. It is to be understood that the AR system may be used for correcting any of the above-mentioned deformities, etc.

During virtual planning, patient data such as medical images (CT, (CB)CT, dental scans, etc.), dento-facial examination (frontal, lateral, oral cavity, TMJ), cephalometric measurements and clinical examination information may be retrieved from the scanning image and image storage module 105 of the AR system. The information is then sent to the virtual-3-D-model-creation module 106 for creating one or more virtual anatomical models. This defines the virtual anatomical 3-D model of a relevant anatomical part. The segmented (CB)CT data is used to create virtual 3-D models. Volume rendering techniques may also be used to create virtual 3-D models. Additionally, patient dentition data (such as intra-oral scans, optical scans of plaster casts, etc.) may also be acquired to create dental 3-D models. The detailed representation of teeth combined with the bone models from the (CB)CT data may be used to create a combined virtual anatomical 3-D model. Optionally, this step may be performed intra-op as well. Additionally, an intra-op C-arm scan may be taken wherein a lateral X-ray is taken of the patient and transformed in an AR readable format.

After the virtual anatomical 3-D model is created, virtual planning using the planning module 108 is performed on the virtual anatomical 3-D model. The planning may be performed pre-op or intra-op. A plurality of actions may be performed during planning such as mirroring, defect identification and, position and orientation of bone cuts for the maxilla and/or the mandible, reconstruction using grafts, etc. The natural head position is set that may be visualized as reference planes. Optionally, repositioning of mandible with condyles in centric relation if not done during medical imaging e.g., at the time of taking a CT or (CB)CT scan may also be performed.

In certain aspects, when planning is performed intra-op, the virtual anatomical 3-D models may be (directly) visualized by the visualization module 110, the natural head position may be visualized on the patient in the form of reference planes, cephalometric landmarks may be visualized in overlay mode on the virtual anatomical 3-D model at the virtual workbench or on the patient directly, etc. Optionally, the virtual anatomical 3-D model may be registered to the patient and overlaid on the patient directly. Additionally, the analysis may be performed on the patient, results of which can be used by the user during virtual planning.

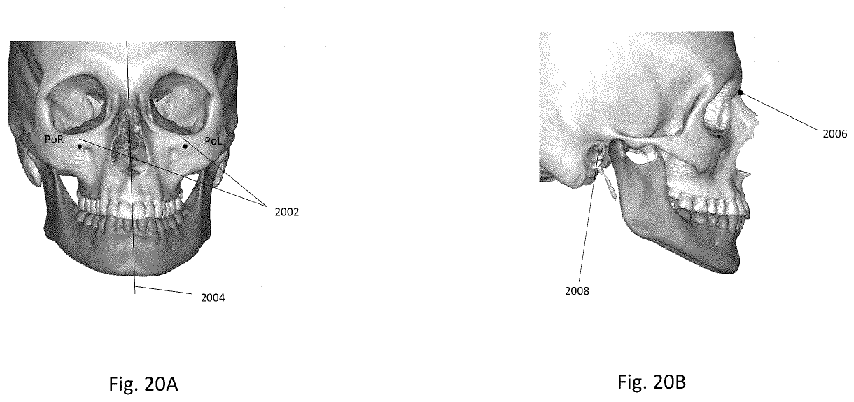

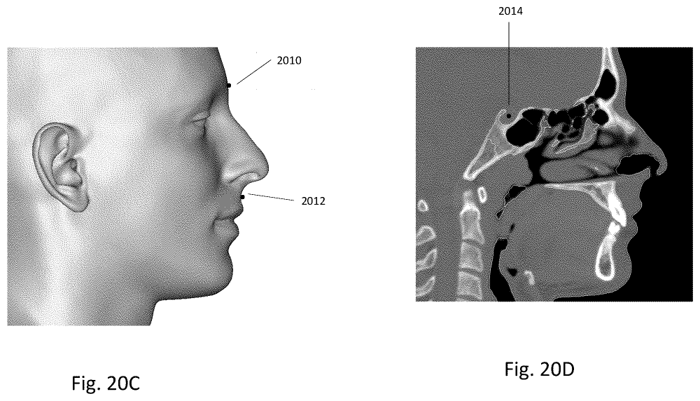

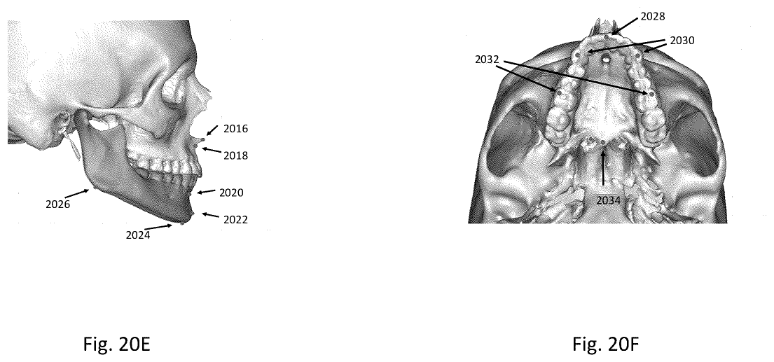

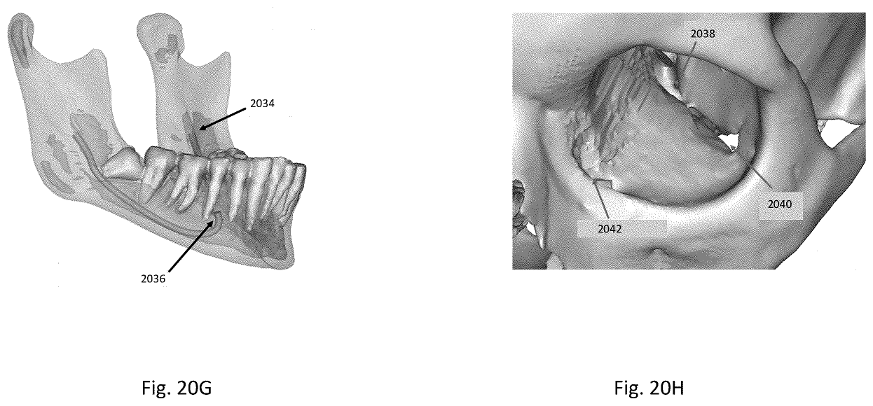

Depending on the surgical procedure, landmarks can be used for either registration/cephalometric indication or tracked within the surgical plan. Figures (see FIGS. 20A-20H) illustrate some examples of both registration and tracked landmarks for orthognathic cases. One or more of these landmarks may be used by the AR system for registration and/or tracking purposes. For example, the orbitale 2002 and the portion 2008 may be used for defining the Frankfurt Horizontal, the midsagittal plane 2004 may be used for establishing the midline and as a symmetry reference during a surgery, the nasion 2006 and the sella turcica 2014 are used for indicating the Steiner Analysis, the glabella 2010 and subnasale 2012 are used as landmarks during bone repositioning, skeletal landmarks (e.g., A point 2018, B point 2020, ANS 2016, PNS 2034, pogonion 2022, menton 2024, gonial angle 2026), dental landmarks (e.g., maxilla or mandible cusps 2028, molar cusps 2032, canine cusps 2023, incisor midlines) may be used for tracking by the AR system. Additionally, the AR system may also show warning signs for critical anatomical structures to avoid such as the mental foramen 2036, the lingula 2034, optic nerve 2038, lacrimal system 2042, inferior fissure 2040, etc. A person skilled in the art will understand which landmarks are to be used during a surgical process and use the AR system to register and track accordingly. A person skilled in the art will also understand that these are exemplary landmarks, and other landmarks may also be used.

Certain embodiments comprise methods of using one or more modules of the AR system for an orthognathic surgery as described herein.

### Reconstruction

Certain embodiments comprise methods of using one or more modules of the AR system during a reconstruction surgery as described herein.

Reconstruction of one or more anatomical parts due to trauma, oncology or congenital defects, fractures, deformities, or disorders. For example, reconstructions of the mandible and/or maxilla are essential for restoring the patient's quality of life, since these anatomies are essential for the masticatory and phonetic functions, support the teeth and define the shape of the lower part of the patient's face. These defects could also impact the breathing of the patient.

Reconstruction surgeries may be done to correct deformities/defects in one or more of below mentioned regions. The list is illustrative and non-exhaustive and other deformities/defects not mentioned herein may also be treated using reconstruction surgeries.

(trigonocephaly),

| | |
|---|---|
| Skull base & | Metopic craniosynostosis (trigonocephaly), sagittal |
| cranial vault | craniosynostosis (scaphocephaly) bilateral coronal |
| | craniosynostosis (brachycephaly), unicoronal coronal |
| | craniosynostosis (anterior plagiocephaly), lambdoid |
| | craniosynostosis (posterior plagiocephaly) |
| Facial nerve | Reversible paralysis, irreversible paralysis (eye complex, midface |
| | and mouth, mouth and lower lip) |
| Nose | Superficial defects (small and/or large), full thickness defect, NOE |
| | involvement |
| Midface | Brown I (palate, alveolus), brown II, brown III, brown IV, |
| | maxillary alveolus, nasal aperture/alar base, piriform rim palate, |
| | zygoma |
| Mandible | Inner cortical, double cortical (full thickness), lateral (with or |
| | without condyle), symphysis (with or without condyle), |
| | parasymphysis, mandible body, angles, rami |
| Orbit | Orbital floor blowout fracture, medial wall, lateral wall, orbital |
| | roof, ZMC fracture |
| Distraction | Mandibular hypoplasia, Treacher collins sydrome, pierre robin |
| Osteogenesis | sequence, goldenhar syndrome, hemifacial microsomia |
| Facial | Not a result a deformity, but an elective & aesthetic procedure that |
| Feminization | seeks to alter the contours of the forehead/brow, midface, mandible |
| | body, gonial angles, chin, or any combination thereof |

Reconstruction surgeries may be performed to reconstruct one or more anatomical parts due to trauma, for example damage caused due to someone being in a car accident, etc. Trauma surgeries may be classified based on the anatomical region that has been damaged. Below is an illustrative and non-exhaustive list of classification of trauma based on deformities/defects/fractures, etc.

| | |
|---|---|
| Mandibular | Body and angle (simple or complex), ramus and condyle, Para |
| | symphysis and symphysis, according to tooth region, condylar neck |
| | fractures |
| Midfacial | Palatoalveolar fracture (simple or comminuted), le fort (I, II, or III), |
| (maxilla, naso- | nasal NOE (NOE, nasal bone), nose (small superficial defect, larger |
| orbital- | partial defect, full thickness), orbit (floor, medial wall, roof, lateral |
| ethmoid, | wall, combined fractures), zygoma (isolatd zygomatic arch |
| zygomatic, | fracture, fracture of the zygomatic complex) |
| orbital, frontal | |
| sinus and | |
| frontal bone, | |
| panfacial) | |
| Skull base & | Cranial skull base, posterior skull base, central (anterior) skull base, |
| cranial vault | lateral skull base, temporal bone, cranial vault |
| Dentoalveolar | Tooth fracture (enamel only, pulp exposure, crown-root, root |
| trauma | fracture), tooth luxation (with or without displacement, avulsion), |
| | alveolar fracture (segmental alveolar fracture) |

Some reconstruction surgeries may also be performed to correct disorders of the temporo-mandibular joint (TMJ) such as condylectomy, total joint replacement, le fort I osteotomy, genioplasty or other types of orthognathic surgeries, menisectomy (removal of the disc) with or without replacement of the disc, etc. Some types of deformities may be due to other medical conditions such as paralysis affecting the CMF region, for example, paralysis of the facial nerve, both reversible and irreversible (eye complex, midface and mouth, mouth and lower lip.

Some reconstruction surgeries may be performed to correct deformities of the CMF region such as of the sequela (skull base and cranial vault, midface or mandible), congenital deformities such as craniosynostosis, orbital hypertelorism, treacher collins syndrome (TCS), cleft lip and palate (CLP), hemifacial microsomia (HFM), fibrous dysplasia, etc. Bone lengthening surgeries may also be performed in some cases such as for one or more anatomical parts of the mandible (e.g., ramus, angle, body) and/or maxilla (e.g., palatal widening).

Some surgical interventions may be carried out due to tumor growth. Tumor growth may be benign or malignant. Tumor may further be classified using the Brown classification into Class I, II, III, or IV such as tumors of the midface.

Illustrative, non-exhaustive list of types of tumors found in the CMF region is given below. A surgeon may be able to identify the correct type of tumor during diagnosis and take that into account during virtual planning, as will be described herein.

| | |
|---|---|
| Malignant | Rhabdosarcomas, condrosarcomas, ewing's sarcoma, osteosarcoma, |
| | malignant ameloblastoma, dermatofibrosarcoma protuberans, |
| | malignant peripheral nerve sheath tumors, Langerhans cell |
| | histiocytosis, ethesioneuroblastoma |
| Benign | Hemangiomas, mehangiopericytomas, myofibromas, osteoma, |
| | osteoblastoma, giant cell granuloma, desmoid tumors, |
| | ameloblastoma, odontogenic tumors, osteochondroma (exostosis), |
| | fibroma, lipomas, enchrondroma, neurofibroma |
| | (neurofibromatosis), fibrous dysplasia, myxoma, odontogenic |
| | kertocysts |

In certain embodiments, the AR system is used during a reconstruction procedure. A typical process of a reconstruction surgery for treating defects/deformities is described herein. It is to be understood that a surgeon may deviate from one or more steps depending on the nature of the surgical procedure.

Certain aspects of the present disclosure are directed towards systems and methods for correcting a defect of a bone structure such as of the orbital floor, medial orbital wall, lateral orbital wall, orbital roof, or combination thereof using AR system. Orbital-floor reconstruction surgeries may be performed to treat conditions such as diplopia (double vision), heterotopia, posterior displacement of the eye (enophthalmos), strabismus, bulging of the eye, or one or more of orbital and zygomatic fractures.

One or more modules of the AR system such as the display device 104, the I/O module 122, scanning-device and image-storage module 105, case management module 120, virtual-3-D-model-creation module 106, planning module 108, visualization module 110, calibration module 112, registration module 114, guidance module 116, control module 124, the virtual workbench may be used individually or as plurality of combinations during an orbital-floor reconstruction surgery.

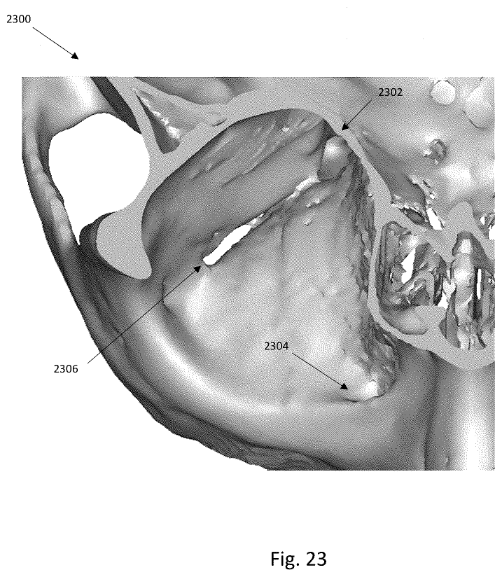

Certain embodiments comprise methods of using one or more modules of the AR system such as planning 108, visualization 110 and/or registration 114 modules during orbital-floor reconstruction surgery. Reference is made to FIG. 23, that illustrates anatomical/cephalometric landmarks that may be used during augmented reality assisted orbital-floor reconstruction surgery. It is to be understood that anatomical landmarks/cephalometric landmarks other than those highlighted in the figure may be used, as described herein. During virtual planning of a surgical procedure of an orbital floor, several regions of the orbital area may be tracked and/or visualized such as the ethmoid bone, lacrimal bone, palatine bone, maxilla, zygomatic and frontal bone. Depending on the defect, anatomical (bony) landmarks such as the remnant posterior shelf, inferior orbital rim, remnant medial/lateral orbit, and overlay of contralateral anatomy (if available) are all visualized when considering optimal reconstruction and implant design. Several muscles may also be visualized such as the infraorbital fissures (2306), oblique muscles, rectus muscles, soft tissue elements such as the inferior oblique, lacrimal system (2304), etc. Important nerves that must be avoided may also be visualized such as the zygomatic nerve, trigeminal nerve, optic nerve (2302), koorneef bag etc.

During the surgery, positioning an implant onto the defect of the bone structure is carried out, wherein the surface of the implant in contact with the bone structure is configured to match (such as the floor and/or the rim of the orbital bone structure). The guidance module 116 of the AR system may be used for positioning the implant. Several warning signs by the AR system may be provided such as warnings to avoid the nerve damage by indicating the location of the optic (2302), trigeminal and/or zygomatic nerve, infraorbital fissure (2306) muscle. One key thing in orbital floor surgery is to make sure that the implant is positioned correctly since mispositioning an implant can have a huge impact on the patient outcome. The AR system may provide warnings during the positioning of an implant to minimize mispositioning. For example, the visualization module 110 may visualize the areas that need to be avoided with the implant contour and display appropriate warning signs when the user is too close to the said areas. Areas to be avoided may comprise of infraorbital fissure (2306) (avoid entering the fissure), when the implant is too close to the optic canal or optic nerve (2302), when the implant may cover more than 65% of the height of the orbit, etc. The registration 114, guidance 116 and/or visualization 110 module(s) may also display screw trajectories during fixation of the implant to make sure that the screw angulation is correct. For example, during transconjunctival surgical approach, the screw is angled perpendicular to the rim, the AR system may guide the user in screw placement by visualizing the screw trajectory and displaying a warning if the user is off the desired trajectory.

Certain aspects of the present disclosure are directed towards systems and methods for repositioning of plurality of bone and/or bone fragments using the AR system. The steps and details of repositioning can vary and may include establishing occlusion, reducing fractures to restore normative anatomy following trauma, achieving symmetrical restoration, or overcorrecting to account for craniofacial deformity. This is achieved in a stepwise fashion by linearly translating and/or rotating bony fragments into the proper position in 3-D space. The method includes virtual planning (by the planning module) using devices such as splints, surgical guides, braces that are tracked using one or more trackers and registered by the registration module, planning of positioning of medical devices such as bone pin, dental clamps, etc. The method also includes registration of anatomical parts using one or more of the known registration techniques such as using known landmarks (teeth, bone, etc.), automatic registration (such as dental arch), using intra-op scanning devices such as CT, X-ray, etc.

One or more modules of the AR system such as the display device 104, the I/O module 122, scanning-device and image-storage module 105, case management module 120, virtual-3-D-model-creation module 106, planning module 108, visualization module 110, calibration module 112, registration module 114, guidance module 116, control module 124, the virtual workbench may be used individually or as plurality of combinations during a repositioning of bone fragments, trauma, oncology, etc surgeries.

Certain embodiments comprise methods directed towards using one or more modules of the AR system during virtual planning (by the planning module 108) of one or more bone fragments of the craniomaxillofacial region and can be used for different surgery types. For example, dental models are registered to a segmented CT scan for orthognathic planning, after which the necessary osteotomies are simulated, and the relevant portions of the skeleton are repositioned in 3-D space according to symmetry, cephalometric norms, and patient specific treatment plan concerns. For reconstruction cases, osteotomies are simulated based on appropriate margins, critical landmarks are noted and accounted for, and the optimal reconstruction and fixation is discussed/simulated. This can include but is not limited to various autologous bone grafts, bone morphogenic protein, particulate bone, or engineered bone matrix. The adequate type of plating and fixation protocols are then finalized. For distraction surgery, osteotomies are simulated, and bone repositioning is similarly completed by repositioning the bone in 3-D space, after which the optimal vector and devices are determined. Cranial vault reconstruction involves again simulating any necessary osteotomies, followed by repositioning to establish normal anatomic contour and address asymmetries. Once the planning is completed, the user may now overlay the virtual anatomical model on the patient which is also live tracked using markers. Alternatively, the user may not overlay the virtual anatomical model on the patient but choose to use it at the virtual workbench during the remainder of the surgery. The user may now perform the planned steps. The guidance module 116 may display the osteotomy plane on the patient to provide guidance. The user performs the osteotomy. The guidance module 116 may then display the preplanned positions of the drill holes on the patient and provide guidance to the user to perform the drilling step. Once the holes are drilled, the guidance module 116 may then display the position of the pre-planned plate and the screws on the patient. Optionally, the visualization module 110 in association with the scanning-device and image-storage module 105, the registration module 114, guidance module 116, calibration module 112, may guide the user to select (by providing visual cues such as highlighting) the correct the plate and the screws from a nearby surgical table and help the user in correctly positioning and fixing the plate and screws to the patient. One or more modules of the AR system may also help the user plan and visualize the distraction path in the OR. For example, the user may be able to plan the distraction path, align the desired path (including the desired outcome) to the anatomy, visualize the distraction path by overlaying the distraction path on the patient anatomy, set up the (tracked) distractor to align with the distractor path, and subsequently perform the steps on the patient. Alternatively, one or more steps of virtual planning may be done pre-op as well.

One or more modules of the AR system such as the display device 104, the I/O module 122, scanning-device and image-storage module 105, case management module 120, virtual-3-D-model-creation module 106, planning module 108, visualization module 110, calibration module 112, registration module 114, guidance module 116, control module 124, the virtual workbench may be used individually or as plurality of combinations during craniosynostosis (cranial vault reconstruction) surgery.

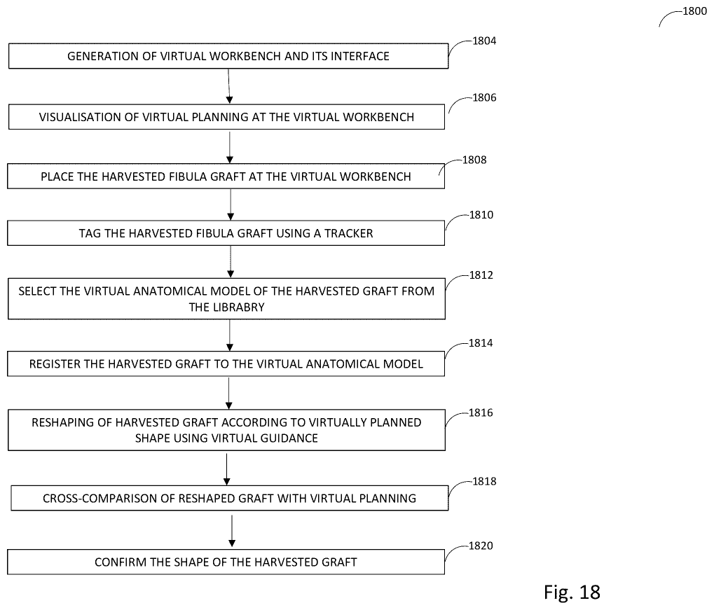

Certain embodiments comprise methods of using the AR system during the process of harvested graft assembly at the virtual workbench, as illustrated in FIG. 18. FIG. 18 illustrates a process 1800 for harvested graft assembly for a reconstruction surgery at the virtual workbench. At step 1804, the surgeon generates the virtual workbench and accesses the planning module 108, such as via a QR code. At step 1806, the virtual planning of the harvested graft assembly is visualized at the virtual workbench. At step 1808, the surgeon also places the harvested fibula graft at the virtual workbench. At step 1810, the harvested (fibula) graft is registered for tracking by the registration module 114 and/or calibration module 112. At step 1812, a virtual anatomic model for a graft (e.g., generic graft model) may be selected from the scanning device or image storage module 105. Alternatively, a virtual model of the harvested graft may be created by the virtual-3-D-model-creation module 106. At step 1814, the harvested graft is then registered to the virtual graft model. At step 1816, the guidance module 116, provides virtual guidance for reshaping the graft. For example, guidance module 116 may highlight areas to be resected by drawing lines or dots or curves or arrows. At step 1818, the surgeon then reviews the reshaped graft against the virtual graft model and either readjusts or confirms the shape. At step 1820, the surgeon confirms the shape of the harvested graft. The reshaped graft is then used during the surgery.

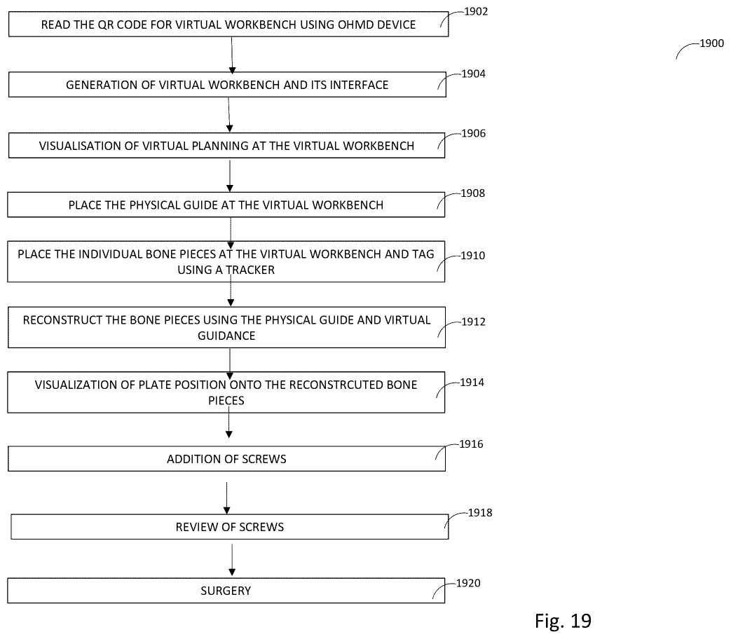

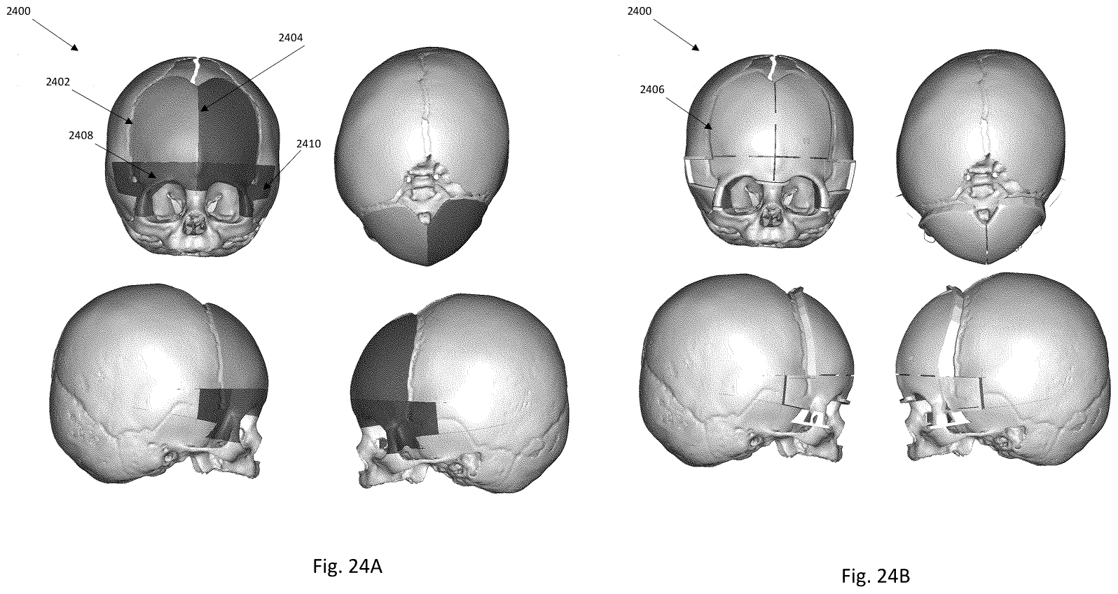

Certain embodiments comprise methods of using the AR system during a craniosynostosis (cranial vault reconstruction) surgery, as illustrated in FIGS. 19, 24A-24B. Craniosynostosis surgery is done to correct premature closure of one or more cranial vault/base sutures (4 main sutures). There are two main types of closures-syndromic and non-syndromic. The sutures are further classified based on the suture involvement such as bilateral coronal (brachycephaly), unilateral coronal (anterior plagiocephaly), unilateral lambdoid (plagiocephaly), metopic (trigonocephaly), sagittal (scaphocephaly) and nonsynostotic posterior plagiocephaly (positional/deformational plagiocephaly). The goal of a craniosynostosis surgery is to reshape the skull to an age appropriate normocephaly and mitigate functional issues. The surgery is normally performed using the coronal (supine, prone or sphinx position) approach.

Reference is made to FIGS. 24A-24B that illustrate anatomical/cephalometric landmarks that may be used during augmented reality assisted craniosynostosis surgery (for example, metopic synostosis). It is to be understood that anatomical landmarks/cephalometric landmarks other than those highlighted in the figure may be used, as described herein. During virtual planning, using scanned medical images from the scanning-device and image-storage module 105, a virtual anatomical model of a patient is created by the virtual-3-D-model-creation module 106. The planning module 108 assists the user in creating a pre-op plan using the virtual anatomical model, including planning osteotomy planes (angulation, position), planning repositioning of the bony segments (2408, 2410, etc) to restore normal anatomy or symmetry, evaluating of total movement, potential gaps and need for bone graft, planning screw placement (position, angulation), planning the type of medical device (resorbable plates) to be used, designing of surgical guides, etc. Once, the pre-op is created, it is stored in the scanning-device and image-storage module 105 or case management module 120. During the surgery, the user may access the pre-op plan via one or more modules of the AR system such as the case management module 120. The user may visualize the planned positions of the osteotomies (2402, 2404, etc.), screw positions, implant, etc. on the virtual anatomical model (2400). The user may also use a physical (e.g., cutting) guide (2406) along with the virtual guidance provided by the guidance module 116. The guidance module 116 may provide step-by-step guidance to the user during the surgery. Normally, surgeons use a physical guide (2406) to mark osteotomy lines on the patient's skull and then performs the osteotomy freehand after the marking is done. Here, the AR system may be used along with the physical guide (2406). The physical guide (2406) may be registered using any known registration techniques and tracked by the registration 114 and calibration 112 modules as described herein. For example, the surgeon may use the physical guide (2406) to mark the osteotomy lines on the skull of a patient. Once marked, the guide is removed. The visualization module 110 at this point may highlight the marked osteotomy lines for better visibility. After the osteotomy is performed, the skull bone is cut into plurality of bone pieces (2408, 2410, etc.). The bone pieces (2408, 2410, etc.) are normally kept aside on a separate surgical table until they're reconstructed. Here, each bone piece (2408, 2410, etc.) may be registered, identified, and tracked (such as shape recognition) for easy reassembly by the registration module 114 by matching it against the pre-op plan. Optionally, each bone piece (2408, 2410, etc.) may be given a specific numerical value (1, 2, or 3) or a color for easy identification based on how it needs to be reassembled. For example, the surgeon may use the virtual workbench for reconstructing the bone pieces (2408, 2410, etc.) together.

Reference is also made to FIG. 19 that illustrates a process of using the virtual workbench for a craniosynostosis surgery. At 1902, the surgeon accesses the virtual workbench, such as using a known marker. At step 1904, the virtual workbench is generated/displayed. At step 1906, the virtual plan is displayed at the virtual workbench. Here, at step 1908, the surgeon may also use a physical guide (e.g., 2406) for reshaping and reconstructing. The physical guide is also tracked and is used at the virtual workbench. For example, at step 1910, the surgeon places the (tracked) plurality of bone pieces on one side of the table. The physical guide is placed in close proximity. The surgeon may pick up the bone piece which is highlighted (for example as 1) by the AR system. At step 1912, the visualization module 110 also highlights the correct position for bone piece 1 on the physical guide. Following the guidance provided by the guidance module 116, the surgeon may reshape (bend) the bone piece 1 to match its corresponding preplanned position. Once the shape and position are matched to the post-op position (which is stored in the database), the AR system confirms it is by notifying the surgeon. This process is repeated iteratively for each bone piece until all the bone pieces are correctly reshaped, assembled and the bone is reconstructed on the physical guide. Next, at step 1914, the AR system highlights the preplanned position of the (resorbable) implant that may be used to hold all the bone pieces together. Alternatively, the surgeon may choose to suture the bone pieces together without using an implant. At step 1916-1918, the surgeon verifies the screws to be used for fixing the implant and reconstructed bones to the patient. The surgeon follows the guidance provided by the guidance module 116 and fixes the implant to the bone pieces and reconstructs the bone. The surgeon then takes the reconstructed bone to be fixed on the patient. The guidance module 116 also provides guidance to fix the reconstructed bone to the patient. This may be done by visualizing the position of the reconstructed bone on the patient.

### EXAMPLE EMBODIMENTS

### Plate Bending

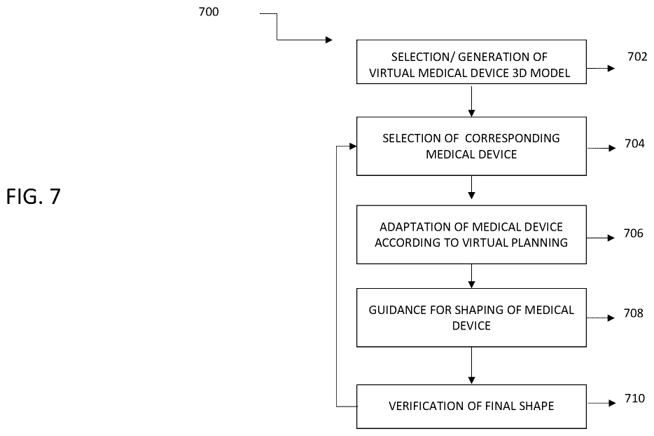

To illustrate as an example, a very simple workflow may be implemented with the AR system of computing environment 100 for adapting a medical device, e.g., shaping (e.g., bending) of a plate. FIG. 7 illustrates a flow chart showing a process 700 of adapting a medical device for an augmented reality system, preferably, plate shaping (e.g., bending), according to certain embodiments.

At blocks 702 and 704, a standard medical device design (such as digital representation of a plate) is selected from a (e.g., digital) library stored in the scanning device and image storage module 105 in accordance with the pre-op plan, physical standard medical device. The selection of medical device to be used during a surgical procedure is based on one or more parameters such as serial number and type of plate, thickness and shape of the plate, type and size of screws, type of fractures (for reconstruction case), etc. Additionally, more than one plate design may also be selected. Alternatively, a plate design may be uploaded either by scanning (part of) packing (QR code, barcode, label) of the actual plate or by using optical recognition techniques of the plate itself.

Virtual medical device 3-D models created by the virtual 3-D model creation module 106 are simulated based on the selected one or more physical plates.

At blocks 706 and 708, the virtual medical device 3-D model may serve as a (virtual) template for guiding the plate shaping (e.g. bending) process. Optionally, the AR system may also allow the user to overlay the virtual medical device 3-D model onto virtual anatomical 3-D model or the actual patient anatomy for guiding the bending process. Further, any difference between the physical plate and the virtual plate may also be highlighted by the AR system. Optionally, the virtual models are registered to the actual physical plates.

Optionally, the plates may also be bent using a robot, whereby the robot is controlled via the control module 124 of the AR system. The robot would take up the function of the bending pliers and be given instructions by the surgeon on how much to bend the plate or alternatively be automatically given instructions by the AR system to bend the plate in order to mimic the shape of the virtual model in a very controlled way avoiding bending too much and requiring partially undoing the bending operation.

The virtual medical device 3-D models may be annotated with adaptation points and optionally, color coded as well. The annotations may be shown using arrows, line, etc. for bending and/or cutting. Additionally, measurements may also be performed on the virtual or actual plates. Alternatively, or additionally, annotations may be overlaid directly on the physical plate as well after registration.

An animation of the step-by-step adaptation guidance by the guidance module 116 is played in the field of view of the AR device, showing each intermediate cutting and/or bending step on the virtual medical device 3-D model. The user follows the step-by-step guidance to bend the actual plate. If the available plate is longer, the AR system will provide guidance (for example, plate trimming). The AR system may virtually show the instruments (such as cutting irons, bending pliers) at the location on the virtual medical device 3-D models (plates) where they may be used. Additionally, the AR system may provide a warning when the cutting iron is positioned at a site where the cut would go through a hole, advising the user to cut at a different location. It may also alert the user when the smooth side of the cutting is positioned at the wrong side.

After trimming, the AR system may indicate the holes where a bending inset needs to be used and guide the user to position them at the right locations. It indicates to the user which threaded plate holes need to be filled with bending insets. In certain aspects, without these insets, the holes become deformed, and the precise seating of the locking screws cannot be guaranteed.

After trimming, the AR system guides the user for bending of the plate. It allows the user to choose the type of bending required such as in-plane bending, out-of-plane bending, torquing, etc. Once the user inputs the type of bending needed, the guidance module 116 of the AR system provides appropriate guidance. Alternatively, or additionally, the AR system may prompt the user to choose between automatic step-by-step guidance or guidance for a particular step. Based on user input, the AR system may then automatically indicate the sequence of bending that is required, including the type of bending used at each step in the sequence. The AR system can guide the user step by step through each individual part of the process. Alternatively, the user may choose a specific step wherein AR guidance is required such as the user requires guidance during the out-of-plate-bending step only. For the remainder of the steps, the guidance may be provided only as visual guidance and not with specific cues.

In case of torquing, the AR system may display the desired end position of the torque, either on the plate itself or (which would be more easily visible) on the instrument position relative to each other or relative to the plates. Angular measurements of the instruments or plate torque (absolute or relative to the target torque) may be shown as indicative to the user in the AR system.

At block 710, the AR system warns the user to conduct quality control checks between the virtually bent plate and the actual plate. The AR system may also perform this check automatically, e.g. by measuring the distance of the physical plate to the virtual template. The AR system may automatically scan the bent plate, or the user may annotate certain landmarks on the plate using a point or hand gestures to determine its shape and difference to the desired template. Hand tracking may be used to scan the plate. This step may be repeated until the shape of the actual plate matches the virtually planned shaped of the plate i.e. until a plate has been reshaped in accordance with the pre-op plan.

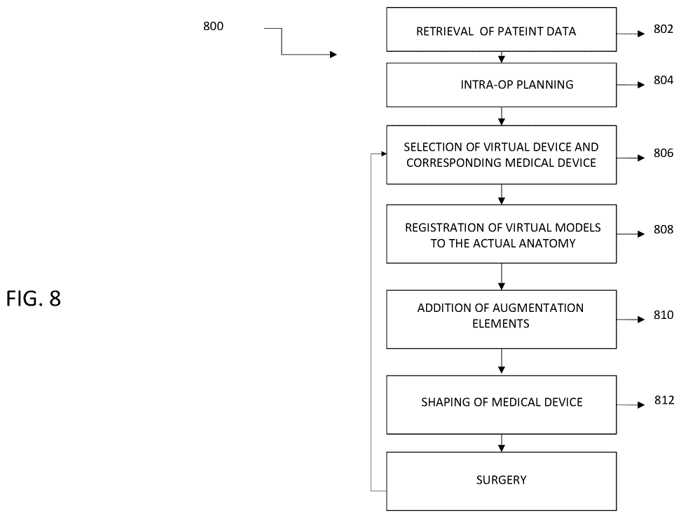

To illustrate as an example, a very simple workflow may be implemented with the AR system of computing environment 100 for bending a plate. FIG. 8 illustrates a flow chart showing a process 800 of adapting a standard medical device into a custom (personalized) device in an augmented reality system for a craniomaxillofacial surgery, according to certain embodiments.

At block 802 patient data (such as medical images) is retrieved from the scanning-device and image-storage module 105 of the AR system at the virtual workbench. The medical images may be used for creating a virtual anatomical 3-D model of a relevant anatomical part by the virtual 3-D model creation module 106 as described herein. Optionally, the virtual anatomical 3-D model may be created beforehand and retrieved from the scanning device and image storage module 105.

At block 804, intra-op planning is done on a virtual anatomical 3-D model, at the virtual workbench. This ranges from mirroring, defect identification such as trauma fragment displacement, osteotomy fragment displacement and, position and orientation of bone cuts for the maxilla and/or the mandible, reconstruction using grafts, etc. Further, other markings and annotations such as lines, curves, or landmarks may also be determined. Optionally, this may be performed intra-op as well.

Alternatively, steps 802-804 may be performed as part of pre-op planning beforehand on a conventional workstation. The results of such pre-op planning may be then stored in the scanning device and image storage module 105, from which it can be retrieved anytime. This way, the surgeon can skip the planning step in the OR.

At block 806, a pre-set target shape or medical device, for example a patient-specific virtual implant design, is selected from the scanning device and image storage module 105 that contains the library (inventory) of medical devices. A ‘best fit’ approach may be used to find the closest match for an implant from the library comprising of standard medical devices.

At block 808, the surgeon registers the virtual 3-D models of anatomy to the patient using a series of pre-defined landmarks that he is annotating with a tracked pointer. One or more physical markers are attached to mandible and maxilla to track the position after registration. The surgeon also registers the virtual 3-D model of the medical device to the physical (actual) device (such as a plate) using a series of pre-defined landmarks or using other recognition to track the position after registration.

At block 810, one or more augmentation elements are added to the reality, such as:
virtual medical device models are overlaid on the actual medical device for visual guidance and quality metric;
the adaption process such as bending and/or cutting is also visualized on the surface of the actual medical device;
the bending angle and the cutting lines are visualized to represent the bending and cutting; and/or
re-joining locations for multiple medical device parts may also be visualized.

At block 812, pliers are used for adapting the medical device (a plate). The pliers may be attached to a physical marker that is tracked by the AR system. The color of the virtual bending positions may be modified according to the angle of the plier in relation to the planned bending position, e.g., red when outside a certain threshold (e.g. 2 degrees), green when inside that threshold. Arrows may be shown interactively to demonstrate how the bending needs to be adapted to create a good alignment.

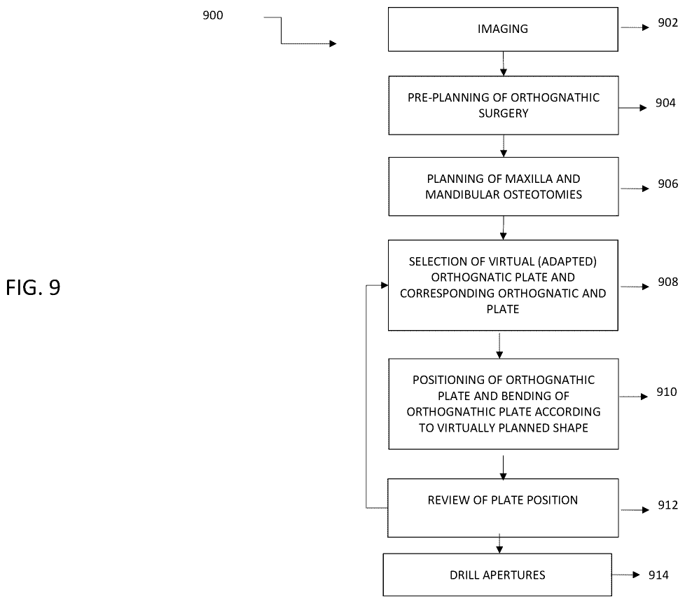

To illustrate as an example, a very simple workflow may be implemented with the AR system of computing environment 100 for adapting a medical device (shaping such as e.g., bending a plate). FIG. 9 illustrates a flow chart showing a process 900 for operating an augmented reality system for shaping (e.g., bending) of a standard plate operating in an augmented reality system for an orthognathic surgery, according to certain embodiments.

At block 902, medical imaging is performed beforehand on a conventional planning workstation. The medical images are used for creating a virtual anatomical 3-D model of a relevant anatomical part as described herein. Additionally, patient dentition data (such as intra-oral scans, optical scans of plaster casts, etc.) may also be acquired to create dental 3-D models. The detailed representation of teeth combined with the bone models from the (CB)CT data may be used to create a combined virtual anatomical 3-D model. Optionally, this step may be performed intra-op as well. Additionally, an intra-op C-arm scan may be taken wherein a lateral X-ray is taken of the patient and transformed in an AR readable format.

At block 904, pre-operative planning of orthognathic surgery is performed on a conventional planning workstation wherein planning is done on a virtual anatomical 3-D model. This ranges from mirroring, defect identification such as trauma fragment displacement, osteotomy fragment displacement and, position and orientation of bone cuts for the maxilla and/or the mandible, reconstruction using grafts, etc. The natural head position is set that may be visualized as reference planes. Optionally, cephalometric landmark points for cephalometric analysis as input may also be planned. Optionally, repositioning of mandible with condyles in centric relation if not done during medical imaging e.g., at the time of taking a CT or CBCT scan may also be performed.

Optionally, this may be performed intra-op as well. During intra-op planning, the virtual 3-D models may be directly visualized in AR by the visualization module 110, the natural head position may be visualized directly on the patient in the form of reference planes, cephalometric landmarks may be visualized in overlay mode on a virtual anatomical model (e.g., at the virtual workbench) or on the patient directly, etc. Further, the virtual anatomical 3-D models may be registered to the patient and overlaid on the patient directly.

At block 906, the surgeon plans the various steps of the surgery plan:

Along with planning for the osteotomy, the surgeon also defines the surgical approach for the osteotomy. Various types of surgical approaches are possible, such as periorbital (lower eyelid, transconjunctival, supraorbital, upper eyelid), coronal, transoral to the facial skeleton (maxillary, mandibular), transfacial to the mandible (submandibular, retromandibular, rhytidectomy), to the TMJ (preauricular). While planning an orthognathic surgery, a surgeon may choose one of or a combination of these approaches such as intraoral (transoral) approach.

### Option 1: Maxilla First Surgery:

The maxillary osteotomies (such as LeFort I, LeFort II or LeFort III) are simulated on the virtual anatomical 3-D model obtained from earlier steps. In the intra-op mode, the surgeon may view the osteotomies directly on the patient or on the virtual anatomical 3-D model. The osteotomies may be indicated as lines, planes, etc. In this step, the maxilla is repositioned to its final position (e.g., the post-op position). During this step, the surgeon may use the cephalometric analysis include a cant evaluation from the infraorbital rim, occlusal plane in relation to Frankfurt Horizontal (FH), Steiner Analysis, mandibular plan angle in relation to FH, Holdaway Ratio, facial thirds, and net movements in the X/Y/Z plane of any or all of the cephalometric landmarks listed in the above sections as input to verify the post-op position against that of a healthy patient. The post-op position may be visualized to the surgeon in combination with pre-op position and in comparison, to that of an average healthy patient, if required by the user. Optionally, the maxilla may be repositioned without making use of any cephalometric data and be verified by the surgeon directly.

Next, the mandibular osteotomies are simulated on the same virtual anatomical 3-D model obtained from earlier steps. In the intra-op mode, the surgeon may view the osteotomies directly on the patient or on the virtual anatomical 3-D model. The osteotomies may be indicated as lines, planes, etc. In this step, the mandible is repositioned to its final position (i.e. the post-op position). The desired occlusion is checked against the planned post-op maxillary teeth. This may be done using an occlusion scan for registering the mandibular teeth in occlusion with maxillary teeth identical to their relative position in the occlusion scan. Thereby registering the occlusion of the maxillary teeth onto the maxillary teeth post-op. The mandibular teeth model is positioned by registering the mandibular teeth on the mandibular part of the occlusion scan. Alternatively, when no occlusion scan is available, the surgeon may manually position the mandibular model until desired occlusion is obtained. Further alternatively, when no occlusion scan is available, the AR system may use its planning algorithm (of the planning module 108) to optimize and suggest the occlusion between the mandibular teeth and the maxillary teeth by allowing the user to indicate certain points and optimize the occlusion automatically based on the input received, or alternatively in a completely automated mode.

### Option 2: Mandible First Surgery

The mandibular osteotomies (BSSO, IVRO or Inverted L) are simulated on the virtual anatomical 3-D model obtained from earlier steps. In the intra-op mode, the surgeon may view the osteotomies directly on the patient or on the virtual anatomical 3-D model (at the virtual workbench). The osteotomies may be indicated as lines, planes, etc. In this step, the mandible is repositioned to its final position (e.g., the post-op position).

Next, the maxillary osteotomies are simulated on the virtual anatomical 3-D model obtained from earlier steps. In the intra-op mode, the surgeon may view the osteotomies directly on the patient or on the virtual anatomical 3-D model (at the virtual workbench). The osteotomies may be indicated as lines, planes, etc. In this step, the maxilla is repositioned to its final position (e.g., the post-op position). The desired occlusion is checked against the planned post-op mandibular teeth. This may be done using an occlusion scan for registering the maxillary teeth in occlusion with mandibular teeth identical to their relative position in the occlusion scan. Thereby registering the occlusion of the mandibular teeth onto the mandibular teeth post-op. The mandibular teeth model is positioned by registering the mandibular teeth on the mandibular part of the occlusion scan. Similarly, the maxillary teeth model is positioned by registering the maxillary teeth on the maxilla part of the occlusion scan. Alternatively, when no occlusion scan is available, the surgeon may manually position the maxillary model until desired occlusion is obtained. Further alternatively, when no occlusion scan is available, the AR system may use its planning algorithm to optimize and suggest the occlusion between the mandibular teeth and the maxillary teeth by allowing the user to indicate certain points and optimize the occlusion automatically based on the input received, or alternatively in a completely automated mode.

The final position of both maxilla and mandible are verified and fine tuning may be done by repetition of one or more of the above-described steps.

### Option 3: Intra-Op C-Scan Step

after the lateral X-ray of the patient is imported in the planning module. The maxillary and mandibular bones are automatically identified by contouring and anatomical landmarks as per surgeon's preferred 2-D cephalometric analysis. This may be performed automatically. The bone contours are automatically repositioned based on normal cephalometric values such as Steiner analysis, SNA angle of around 82, etc. The surgeon may review the plan and optionally, fine tune the planned jaw positions. The final jaw positions may be overlaid on the patient for visualizing the post-op result.

At block 908, based on surgical plan, a plate and/or screw selection is carried out.

After registration, the surgical plan, the virtual models are transferred to the augmented environment. Here, one or more augmentation elements are added to the reality, such as:
Virtual bone models are overlaid semi-transparently on the actual anatomy as a visual reference and quality metric,
virtual medical device models are overlaid semi-transparently on the actual medical device as a visual reference and quality metric;
planned apertures are visualized as tunnels or drill cylinders on the surface of the intra-operative maxilla or mandibular bone or both; and/or
a semi-transparent plane is visualized to represent the depth of the aperture (or tunnel), taking into account the occlusion of the bones (the plane is only shown outside the volume of the bones).

The plate and/or screws are selected and positioned for fixation of bone segments. The following steps may need to be repeated for each fixation of two bone segments in the mandible and/or the maxilla.

Standard plate(s) such as L plate, I plate, pre-bent maxillary plate, adaption plate, curved sagittal split plate, straight sagittal split plate, double bend chin plate selected from the medical device inventory (or library) stored in the image scanning and image storage module 105 may be selected. The plate(s) selected from scanning device or image storage module 105 corresponds to the actual plate(s) that the surgeon plans to use during the surgery. Alternatively, the library may intuitively only show the plates that are indicated for the specific jawbone, and/or show the plates according to the surgeon's preference (e.g., the surgeon's most used/favourite plates for the specific indication, the surgeon's preferred order of presenting the plate), or show only the plates that are readily available at the hospital.

After selection, at block 910, the plate may be positioned and adapted using one of the following approaches. A combination of one or more approaches may also be used.

### Option 1: Manual Positioning

Using the virtual anatomical 3-D model, the plate may be positioned on top of the bone segments in approximate location such that the plate is correctly positioned across the osteotomy gap.

Additionally, a tool such as plate bending tool that guides bending of the plate and fitting it to the specific bone anatomy while respecting a smooth shape, without introducing any sharp bends, and while maximizing the contact with the bone in general or for specific areas may be used (e.g., area around screw holes, area close to the osteotomy, etc.).

The surgeon can review the final position and fine tune the position if needed. Any changes made to the position of the plate are also reflected onto the virtual anatomical 3-D model in terms of fit/adaptation.

The surgeon can review the final shape and locally adapt the shape if needed, such as at the virtual workbench. Additionally, the surgeon may verify the adapted plate against the virtual anatomical 3-D model and against a virtual model of the plate to verify bending. The surgeon may cut the plate extensions, if needed. Alternatively, for extra guidance the surgeon may also use a generic virtual medical device model from the inventory along with the use of the virtual anatomical 3-D model. Alternatively, the surgeon may choose to only work with the virtual medical device 3-D model during adaptation and only perform the final verification step using the virtual anatomical 3-D model.

### Option 2: Manual Positioning

Using the virtual anatomical 3-D model, the plate may be positioned by positioning the left/right end of the plate at the desired location of the left/right bone segment.

Additionally, a tool that guides bending of the plate and fitting it to the specific bone anatomy while respecting a smooth shape, without introducing any sharp bends, and while maximizing the contact with the bone in general or for specific areas may be used (e.g., area around screw holes, area close to the osteotomy, etc.).

The surgeon can review the final position and fine tune the position if needed, such as at the virtual workbench. Any changes made to the position of the plate are also reflected onto the virtual anatomical 3-D model in terms of fit/adaptation.

The surgeon can review the final shape and locally adapt the shape if needed. Additionally, the surgeon may verify the adapted plate against the virtual anatomical 3-D model and against a virtual 3-D model of the plate to verify bending. The surgeon may cut the plate extensions, if needed.

Alternatively, for extra guidance the surgeon may also use a generic virtual medical device model from the inventory along with the use of the virtual anatomical 3-D model. Alternatively, the surgeon may choose to only work with the virtual medical device 3-D model during adaptation and only perform the final verification step using the virtual anatomical 3-D model.

### Option 3: Semi-Automated Positioning

Using the virtual anatomical 3-D model, indicate/mark the area on both bone segments on which the plate should be fitted. This may be done by, for example drawing a contour, or coloring a bone part, or indicating approximate position of screw fixation holes, or by approximately placing the unbent standard plate.

The AR system, based on this information, may automatically position the plate and virtually bend the plate to fit on the specific bone anatomy as seen on the virtual anatomical 3-D model,

Additionally, a tool that guides bending of the plate and fitting it to the specific bone anatomy while respecting a smooth shape, without introducing any sharp bends, and while maximizing the contact with the bone in general or for specific areas may be used (e.g., are around screw holes, area close to the osteotomy, etc.). The provided marking may also allow the AR system to cut the plate to the desired length, if needed.

The surgeon can review the final position and fine tune the position if needed. Any changes made to the position of the plate are also reflected onto the virtual anatomical 3-D model in terms of fit/adaptation.

The surgeon can review the final shape and locally adapt the shape if needed, such as at the virtual workbench. Additionally, the surgeon may verify the adapted plate against the virtual anatomical 3-D model and against a virtual model of the plate to verify bending. The surgeon may fine tune the plate, by either extending or shortening the plate. Alternatively, for extra guidance the surgeon may also use a generic virtual medical device model from the inventory along with the use of the virtual anatomical 3-D model. Alternatively, the surgeon may choose to only work with the virtual medical device 3-D model during adaptation and only perform the final verification step using the virtual anatomical 3-D model.

### Option 4: Automated Positioning

Using the virtual anatomical 3-D model, indicate the osteotomy that should be fixated with the selected plate from the inventory.

The AR system may automatically position the plate across the osteotomy while taking into account the bone quality for the screw fixation holes, surgeon preferences or both. The bone quality may be reflected on the virtual anatomical 3-D model in the form of different color or a color gradient or other features as described earlier.

The AR system may automatically propose cutting of the plate whenever this seems beneficial.

### Review of Plate

Color mapping may be used to show the distance between plate and bone on the virtual anatomical 3-D model for review of fit/position. Color mapping may be linked to min and max values as specified in preferences and only show values outside these threshold values.

At block 912, additionally, plate position may be reviewed with respect to critical anatomical structures, e.g., distance to nerve, distance to tooth root, etc., by overlaying critical anatomical structures on the virtual anatomical 3-D model. Alternatively, or additionally, safety margins according to values set in preferences may be visualized.

### Addition of Screws

Option 1: Manual positioning-A standard screw is selected from the medical device inventory (or library) stored in the image scanning and image storage module 105 based on parameters such as desired type, diameter, length, etc. Alternatively, the library may intuitively only show the screws that are indicated for the specific jawbone, and/or show the screws according to the surgeon's preference (e.g., the surgeon's most used/favorite screws for the specific indication, the surgeon's preferred order of presenting the screw, or show only the screws that are readily available at the hospital.

Using a virtual medical device 3-D model, the fixation hole in the plate in which the screw needs to be placed may be indicated.

The AR system may correctly position the screws inside the bone plate fixation hole, e.g., align the axis of the screw with the axis of the fixation hole in the plate and position the head of the screw in contact with the plate. The surgeon may review the length of the screw and switch lengths, if needed. The surgeon may also review the angulation (around 15 degrees) of the screw in relation to the surrounding bone quality and optionally angulate the screw to be seated in higher quality bone but restricted to maximal allowed angulation with respect to the plate hole. The surgeon may review this using the virtual anatomical 3-D model or on the patient directly.

### Option 2: Semi-Automated Positioning

A standard screw is selected from the medical device inventory (or library) stored in the image scanning and image storage module 105 based on parameters such as desired type, diameter, length, etc. Alternatively, the library may intuitively only show the screws that are indicated for the specific jawbone, and/or show the screws according to the surgeon's preference (e.g., the surgeon's most used/favorite screws for the specific indication, the surgeon's preferred order of presenting the screw, or show only the screws that are readily available at the hospital.

The AR system may automatically position minimally 2 screws per segment in the locations that have the best bone quality. The surgeon may review the length of the screw and switch lengths, if needed. The surgeon may fine tune by removing or adding a screw. However, the AR system may give a warning if a minimum of 2 screws per segment is not respected, as per standard protocol.

### Option 3: Fully Automated Positioning

The AR system may automatically select a screw from the medical device library based on one or more of the following criteria: default screw for type of application, surgeon preferred screws for this type of application, most suitable screw (diameter, length) based on bone quality for this type of application, show only the screws that are readily available at the hospital.

The AR system may automatically position minimally 2 screws per segment in the locations that have the best bone quality.

The surgeon may review the length of the screw and switch lengths, if needed. The surgeon may fine tune by removing or adding a screw. However, the AR system may give a warning if a minimum of 2 screws per segment is not respected, as per standard protocol.

### Review of Screws

Screw position may be reviewed with respect to critical anatomical structures, e.g., distance to nerve, distance to tooth root, etc., by overlaying critical anatomical structures on the virtual anatomical 3-D model. Alternatively, or additionally, safety margins according to values set in preferences may be visualized.

At block 914, a drill machine is used to make the apertures. The drill machine itself may be attached to a physical marker that is tracked by the AR system. The drill machine may be augmented by recoloring it along the axis as markings: red=not within a 1 mm, Green=within a 1-2 mm. The apertures are made. In certain embodiments, the system detects the drill machine's position in the bone and gives a warning signal when the drilling distance exceeds the optimum (pre-planned) depth to avoid loss of bone stock. Additionally, this may be followed for performing osteotomies as well.

The AR system may not be used for the remainder of the procedure.

In other embodiments, the procedure may be planned intra-operatively in its entirety wherein the surgeon takes with him/her medical images and 3-D virtual models using the virtual 3-D model creation module 106 in the OR and plans the remainder of the surgery in real time using the augmented reality system.

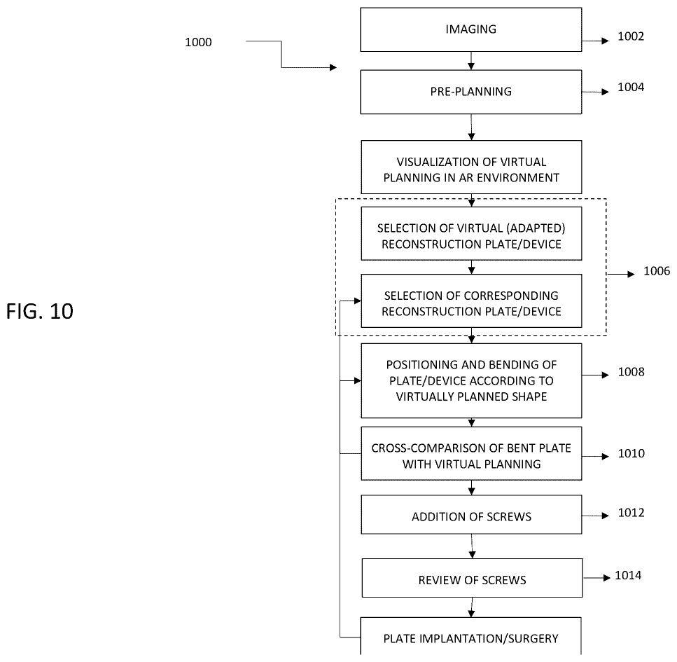

FIG. 10 illustrates a flow chart showing a process 1000 of bending of a standard plate operating in an augmented reality system for a reconstruction surgery of a mandible, according to certain embodiments.

At block 1002, medical imaging is performed beforehand on a conventional planning workstation. Medical images are used for creating the virtual anatomical 3-D model of a relevant anatomical part (e.g., the mandible) as described herein. Additionally, patient dentition data (such as intra-oral scans, optical scans of plaster casts, etc.) may also be acquired to create dental 3-D models. The detailed representation of teeth combined with the bone models from the (CB)CT data may be used to create a combined virtual anatomical 3-D model. The CT data may also include information of a patient specific fibula graft. A virtual anatomical 3-D model of the fibula graft may also be generated. Optionally, a generic fibular graft may also be visualized.

Optionally, this step may be performed intra-op as well. Alternatively, an intra-op C-arm scan may be taken wherein a lateral X-ray is taken of the patient and transformed in an AR readable format.

At block 1004, pre-operative planning is performed on a conventional planning workstation wherein planning is done on a virtual anatomical 3-D model. This ranges from mirroring, defect identification such as trauma fragment displacement, osteotomy fragment displacement and, position and orientation of bone cuts for the maxilla and/or the mandible, reconstruction using grafts, etc. The natural head position is set that may be visualized as reference planes. Optionally, cephalometric landmark points for cephalometric analysis as input may also be planned. Optionally, repositioning of mandible with condyles in centric relation if not done during medical imaging e.g., at the time of taking a CT or CBCT scan may also be performed.

Optionally, this may be performed intra-op as well. During intra-op planning, the virtual 3-D models may be directly visualized in AR, the natural head position may be visualized directly on the patient in the form of reference planes, cephalometric landmarks may be visualized in overlay mode on a virtual anatomical model or on the patient directly, etc. Further, the virtual anatomical 3-D models may be registered to the patient and overlaid on the patient directly.

The surgeon plans the various steps of the surgery plan:

Planning and simulation of osteotomies for native mandible that may require reconstruction. In case of tumor reconstruction, first the osteotomies on the native bone are defined based on the necessary resection of the tumor.

Planning of the mandibular reconstruction—In case of an unilateral defect the reconstruction can be planned based on mirroring the healthy side of the bone to the affected side to guide the final shape of the mandible. Alternatively healthy images of the patient from before the disease-if available-could be used. Alternatively, a generic mandible (e.g., SSM based model) is aligned with the patient's 3-D model of the mandible as a guidance for the reconstruction. Alternatively, the reconstruction is done by manually drawing the desired bone volume taken into account cephalometric landmarks and analyses. Alternatively, the planning is done based on the desired prosthetic outcome, e.g., a prosthetically driven backward planning, In this case prosthetic reconstruction is visualized to restore masticatory and phonetic functions for the patient. Based on this the position of dental implants is defined and further the bone reconstruction. Above-mentioned methods of SSM and cephalometry can be applied in this case as well. In case of non-unilateral defects all above options except mirroring can be applied for planning the reconstruction.

Planning of osteotomies and segments on fibula graft for mandible reconstruction. Based on the planned mandibular bone the fibula graft segments are optimally chosen to mimic the desired shape as good as possible. This can be done in a manual, semi-automated or automated way.

Visualization and fine tuning of mandibular reconstruction. The reconstruction with the fibula segments is visualized in the system and tools are provided to the surgeon to fine tune size, position, and orientation of the fibula segments and as such optimize the reconstruction according to his/her preferences.

At block 1006, based on surgical plan, a plate and/or screw selection is carried out. The plate and/or screws are selected and positioned for fixation of bone segments. The following steps need to be repeated for each fixation of two bone segments in the mandible.

A standard plate is selected from the medical device inventory (or library) stored in the image scanning/image storage module. Alternatively, the library may intuitively only show the plates that are indicated for the specific jawbone, and/or show the plates according to the surgeon's preference (e.g., the surgeon's most used/favorite plates for the specific indication), the surgeon's preferred order of presenting the plate, or show only the plates that are readily available at the hospital.

After selection, at block 1008, the plate may be positioned and adapted using one of the following approaches. A combination of one or more approaches may also be used.

### Option 1: Manual Positioning

Using the virtual anatomical 3-D model, the plate may be positioned on top of the bone segments in approximate location such that the plate is correctly positioned across the osteotomy gap or the contact plane between 2 neighboring bone segments.

Additionally, a tool that guides shaping (e.g., bending) of the plate and fitting it to the specific bone anatomy while respecting a smooth shape, without introducing any sharp bends, and while maximizing the contact with the bone in general or for specific areas may be used (e.g., are around screw holes, area close to the osteotomy . . . ).

The surgeon can review the final position and fine tune the position if needed. Any changes made to the position of the plate are also reflected onto the virtual anatomical 3-D model in terms of fit/adaptation.

The surgeon can review the final shape and locally adapt the shape if needed. Additionally, the surgeon may verify the adapted plate against the virtual anatomical 3-D model and against a virtual model of the plate to verify shaping (e.g., bending). The surgeon may cut the plate extensions, if needed. Alternatively, for extra guidance the surgeon may also use a generic virtual medical device model from the inventory along with the use of the virtual anatomical 3-D model. Alternatively, the surgeon may choose to only work with the virtual medical device 3-D model during adaptation and only perform the final verification step using the virtual anatomical 3-D model.

### Option 2: Manual Positioning

Using the virtual anatomical 3-D model, the plate may be positioned by positioning the left/right end of the plate at the desired location of the left/right bone segment.

Additionally, a tool that guides shaping (e.g., bending) of the plate and fitting it to the specific bone anatomy while respecting a smooth shape, without introducing any sharp bends, and while maximizing the contact with the bone in general or for specific areas may be used (e.g., are around screw holes, area close to the osteotomy, etc.).

The surgeon can review the final position and fine tune the position if needed. Any changes made to the position of the plate are also reflected onto the virtual anatomical 3-D model in terms of fit/adaptation.

The surgeon can review the final shape and locally adapt the shape if needed. Additionally, the surgeon may verify the adapted plate against the virtual anatomical 3-D model and against a virtual model of the plate to verify bending. The surgeon may cut the plate extensions, if needed.

Alternatively, for extra guidance the surgeon may also use a generic virtual medical device model from the inventory along with the use of the virtual anatomical 3-D model. Alternatively, the surgeon may choose to only work with the virtual medical device 3-D model during adaptation and only perform the final verification step using the virtual anatomical 3-D model.

### Option 3: Semi-Automated Positioning

Using the virtual anatomical 3-D model, indicate/mark the area on both bone segments on which the plate should be fitted. This may be done by, for example drawing a contour, or coloring a bone part, or indicating approximate position of screw fixation holes, or by approximately placing the original standard plate (i.e. before shaping (e.g., bending or cutting).

The AR system, based on this information, may automatically position the plate and virtually shape (e.g., bend) the plate to fit on the specific bone anatomy as seen on the virtual anatomical 3-D model,

Additionally, a tool that guides shaping (e.g., bending) of the plate and fitting it to the specific bone anatomy while respecting a smooth shape, without introducing any sharp bends, and while maximizing the contact with the bone in general or for specific areas may be used (e.g., are around screw holes, area close to the osteotomy . . . ). The provided marking may also allow the AR system to cut the plate to the desired length-if needed.

The surgeon can review the final position and fine tune the position if needed. Any changes made to the position of the plate are also reflected onto the virtual anatomical 3-D model in terms of fit/adaptation.

The surgeon can review the final shape and locally adapt the shape if needed. Additionally, the surgeon may verify the adapted plate against the virtual anatomical 3-D model and against a virtual model of the plate to verify bending. The surgeon may fine tune the plate, by either extending or shortening the plate. Alternatively, for extra guidance the surgeon may also use a generic virtual medical device model from the inventory along with the use of the virtual anatomical 3-D model. Alternatively, the surgeon may choose to only work with the virtual medical device 3-D model during adaptation and only perform the final verification step using the virtual anatomical 3-D model.

### Option 4: Automated Positioning

Using the virtual anatomical 3-D model, indicate the osteotomy or the 2 neighboring bone segments that should be fixated with the selected plate from the inventory. Alternatively, the system is fully automatic e.g., without any input from the user for selection of the plate and the bone segments requiring fixation.

The AR system may automatically position the plate across the osteotomy while taking into account the bone quality for the screw fixation holes, surgeon preferences or both. The bone quality may be reflected on the virtual anatomical 3-D model in the form of different color or a color gradient or other features as described earlier.

The AR system may automatically propose cutting of the plate whenever this seems beneficial.

### Review of Plate

At block 1010, color mapping may be used to show the distance between plate and bone on the virtual anatomical 3-D model for review of fit/position. Color mapping may be linked to min and max values as specified in preferences and only show values outside these threshold values.

Additionally, plate position maybe reviewed with respect to critical anatomical structures, e.g., distance to nerve, distance to tooth root, etc., by overlaying critical anatomical structures on the virtual anatomical 3-D model. Alternatively, or additionally, safety margins according to values set in preferences may be visualized.

At block 1012, screws are added

### Option 1: Manual Positioning

A standard screw is selected from the medical device inventory (or library) stored in the image scanning/image storage module based on parameters such as desired type, diameter, length, etc. Alternatively, the library may intuitively only show the screws that are indicated for the specific jawbone, and/or show the screws according to the surgeon's preference (e.g., the surgeon's most used/favorite screws for the specific indication, the surgeon's preferred order of presenting the screw, or show only the screws that are readily available at the hospital).

Using a virtual medical device 3-D model, fixation hole in the plate in which the screw needs to be placed may be indicated.

The AR system may correctly position the screws inside the bone plate fixation hole, i.e. align the axis of the screw with the axis of the fixation hole in the plate and position the head of the screw in contact with the plate. The surgeon may review the length of the screw and switch lengths, if needed. The surgeon may also review the angulation of the screw in relation to the surrounding bone quality and optionally angulate the screw to be seated in higher quality bone but restricted to maximal allowed angulation with respect to the plate hole. The surgeon may review this using the virtual anatomical 3-D model or on the patient directly.

### Option 2: Semi-Automated Positioning

A standard screw is selected from the medical device inventory (or library) stored in the image scanning/image storage module based on parameters such as desired type, diameter, length, etc. Alternatively, the library may intuitively only show the screws that are indicated for the specific jawbone, and/or show the screws according to the surgeon's preference (e.g., the surgeon's most used/favorite screws for the specific indication, the surgeon's preferred order of presenting the screw, or show only the screws that are readily available at the hospital.

The AR system may automatically position minimally 2 screws per segment in the locations that have the best bone quality. The surgeon may review the length of the screw and switch lengths, if needed. The surgeon may fine tune by removing or adding a screw. However, the AR system may give a warning if a minimum of 2 screws per segment is not respected, as per standard protocol.

### Option 3: Fully Automated Positioning

The AR system may automatically select a screw from the medical device library based on one or more of the following criteria: default screw for type of application, surgeon preferred screws for this type of application, most suitable screw (diameter, length) based on bone quality for this type of application, show only the screws that are readily available at the hospital.

The AR system may automatically position minimally 2 screws per segment in the locations that have the best bone quality.

The surgeon may review the length of the screw and switch lengths, if needed. The surgeon may fine tune by removing or adding a screw. However, the AR system may give a warning if a minimum of 2 screws per segment is not respected, as per standard protocol.

### Review of Screws

At block 1014, screw position maybe reviewed with respect to critical anatomical structures, e.g., distance to nerve, distance to tooth root, etc., and with respect to distance to the osteotomy, by overlaying critical anatomical structures on the virtual anatomical 3-D model. Alternatively, or additionally, safety margins according to values set in preferences may be visualized.

The process 1000 may be similarly carried out for reconstructing of other anatomical parts such as maxilla as well.

### Virtual Workbench

Referring to FIG. 12A, an example embodiment of a virtual workbench in a physical environment (OR) 1200 is shown. A virtual workbench is generated at affixed, but adjustable location such as a surgical table 1214. A QR code (not shown) may be placed on the top of the surgical table at 1214. When user 1212 wearing an OHMD device (not shown) stands facing the surgical table 1214, the camera embedded in his OHMD device, generates the virtual workbench 1210, such as based on image recognition of the surgical table 1214, the QR code, or other techniques discussed. A virtual workbench is a three-dimensional entity that occupies space and volume and is visible to the user 1212 via his OHMD device. A user may access the information stored and/or registered by the AR system at the virtual workbench 1210. The border of the virtual workbench 1210 shown in FIG. 12A is for representative purposes only. The user may interact with the virtual workbench using any known AR communication methods described herein such as voice commands, gestures, hand signals, etc. An optional spotlight 1224 is present at the location 1214 to easily guide a user 1212 to the virtual workbench 1210. Other users 1216, 1218, 1220 present in the OR, may continue operating on a patient 1222 at the operating table while user 1212 guides/assists them during the surgery by using information from virtual workbench 1210. Any user that may have access to the AR system can operate the virtual workbench 1210. In certain embodiments, only one user 1212 may have access to the AR system during a surgery in an OR. In that case, user 1212 can access the virtual workbench 1210 at its location 1214 and go back to the operating table with the patient 1222 at any point in time. User 1212 has access to virtual workbench 1210 as long as his OHMD device can recognize the QR code or by simply standing facing the virtual workbench 1210 at its designated location 1214. In the OR, other standard medical equipment 1226 may also be present such as heart monitoring device, medical imaging systems or additional screens. Optionally, the standard medical equipment 1226 can be connected to the AR system via a common network and its data may be accessed by a user 1212 at the virtual workbench 1210.

In FIG. 12B, an example embodiment of a virtual workbench in a physical environment 1200 is shown with an external system 1230 integrated in the AR system. An external system 1230 such as an additive-manufacturing device (3-D printer) may be present in the OR. The external system 1230 is integrated in the AR system and can be controlled by a user 1212 via the virtual workbench 1210. This is helpful in instances when there is a shortage of a certain item in an OR that is also 3-D printable. For example, the users 1218, 1216, 1220 may require additional surgical tags during a surgical procedure. In a normal scenario, a user would have to step out of the OR to retrieve the missing item and scrub back in on his return as the OR is a sterile environment. However, if an external device 1230 (3-D printer) is present in the OR, a user can print the missing item without having to step out of the OR. As the external system 1230 can be controlled virtually, there is no need for a user 1212 to go through scrubbing. Once a missing item is printed, it is ready for use after minimal cleaning as the external system 1230 is located in a sterile environment. It also leads to minimum interruption of the surgical workflow as the users can continue with their surgery while the external system 1230 continues to 3-D print the missing item in the background. Once the printing is finished, a user is notified via the virtual workbench 1210.

In FIG. 12C, an example embodiment of a virtual workbench in a physical environment 1200 is shown with at least two external systems 1230 and 1232 integrated in the AR system. External system 1230 is an additive manufacturing system as described above. An additional external system present in the OR is a robotic arm 1232. The robotic arm 1232 is connected to the AR system via a common network and can be controlled via the virtual workbench 1210. In some cases, a surgeon may like additional help during a surgical procedure and may wish to use a robotic arm 1232 during a surgical procedure. For example, a surgical may wish to use a robotic arm 1232 for cutting a mandible during an orthognathic surgery on patient 1222. User 1212 can control the robotic arm 1232 via the virtual workbench 1210. A three-dimensional virtual model of a robotic arm 1232 is displayed at the virtual workbench 1210. Any actions performed by user 1212 on the virtual model of the robotic arm 1232 at the virtual workbench 1210 is translated to the actual robotic arm 1232 present near the patient 1222. User 1212 may perform actions on the virtual model of the robotic arm using any known communication methods such as hand-based gestures or (haptic) hand movements. This way, a user can provide his input from a distance to the ongoing surgery. This also avoids overcrowding around the patient 1222 and enable experts (such as surgeons with experience in handling robots) to provide their input from a distance.

Referring to FIG. 12D, an example embodiment of a top view of the virtual workbench at its location, as seen by a user via his OHMD device is shown. A marker 1226, such as in the form of a QR code, is shown on the top of the surgical table 1214. The location of where a virtual workbench 1210 will be generated is shown. In this example, the surgical table 1214 also has room for placement of other items such as actual, physical surgical tools (not shown) (such as scissors, pliers, plates, guides, etc). Each of the physical surgical instruments can additionally be marked using separate markers and tracked. The information of said physical surgical instruments can be accessed at the virtual workbench 1210.

Various modules of the AR system such as the registration module, the planning module, calibration module, display module, creation module, printing module etc., relay the information to the virtual workbench where it is accessible at all times to all the connected users of the AR system.

For certain embodiments, reference is made to FIG. 13A-13B. An example GUI of a virtual workbench comprises of at least one virtual pane 1324 visible in/via the OHMD device (not shown) to the user 1312. The virtual pane comprises of a live view 1322 of the video stream in real time captured by the camera of the OHMD device. In certain aspects, the live view pane 1322 covers more than 50% of the area that is visible to the user 1312 in the OHMD device. A plurality of actions is available to a user via at least one tab 1320 for each of a plurality of types of actions to be performed. One or more tabs may be available. Each tab corresponds to a set of actions that may be carried out by interacting with the AR system using any known form of communication method as described herein (touch, voice, gestures, etc.). One of the tabs from the plurality of tabs is ‘clear all’ tab that allows the user to return to home screen at any given point.

The borders of each element that make up the GUI 1300 are for representative purposes. One can imagine that the tabs 1320 are positioned such that they are visible at all times but, for example, take up less than 10% of the space of the GUI. In the example embodiment of FIG. 13A, the plurality of tabs is positioned at the left-hand side of the virtual pane 1324. It is to be understood, that the positioning of the plurality of tabs may be changed, such that it may be positioned on the left or right of the virtual pane 1324. The plurality of tabs is completely customizable in accordance with user preference. On the right-hand side, there is an account tab through which one may access the account of the user 1312. At the bottom of the virtual pane 1324, a tab 1318 displaying the current location in the surgical workflow is provided. This allows the user 1312 to not only track how far along he is in a surgical procedure but may also use it to navigate to that particular step in the surgical workflow, if needed. The GUI 1300 of the virtual workbench 1316 is very simple to use.

Referring to FIG. 13C for yet another example embodiment. The plurality of tabs 1320 have assigned actions that the user 1312 may perform. The user 1312 may interact with the tabs 1320 by selecting a tab using any know communication mediums such as a single click, a single tab or voice command, etc. The GUI 1300 is completely interactive, for example, the user 1312 may interact with the virtual anatomical model 1326, at the virtual work bench 1316. Each tab 1320 has a plurality of related sub-tabs with sub-actions assigned (not shown).

Referring to FIG. 14A, an example embodiment of a GUI 1400 of a virtual workbench when a view tab is selected from a plurality of tabs 1420 is shown. The view tab allows the user to view any information that is stored in one or more modules or databases, such as the pre-op plan, the medical device inventory, one or more virtual 3-D models of the patient, the anatomy, or the medical devices or parts thereof. Thus, the user may use the view tab for reference at any time during the surgery. Once the view tab is selected (by clicking), the virtual pane 1424 is split into at least two, where the second virtual pane displays the sub-tabs 1426 of the view tab. The user may easily select one or more actions from the sub-tabs 1426 by simply clicking on the toggle on/off icon next to the action. The virtual pane comprising of the sub-tabs 1426 is temporarily displayed. Once, the user is done selecting the actions from the sub-tabs 1426, the virtual pane with the sub-tabs collapses and the virtual pane 1428 is back in full screen mode. The virtual pane with the sub-tabs 1426 can be accessed anytime using the collapse tab 1432. Optionally, the user may also decide to export one or more reference steps or information to their OHMD from the virtual workbench to be overlaid around the patient using the export tab 1430. Any user connected to the AR system may access the view tab by standing facing the virtual workbench spot/location. The view tab also provides the user the option to take screenshots that they may then transfer (or export) to their OHMD to assist them during the rest of the surgery. The view tab provides the user viewing tools such as magnifying glass for zoom, etc. Upon selecting the appropriate tool, the action is performed on the virtual anatomical model displayed in the live virtual pane.

In certain embodiments, the user may wish to use the edit tab from a plurality of tabs 1420, as shown in FIG. 14B. The user may wish to edit pre-op plans virtually. The GUI of virtual workbench 1400 will display the option to edit information on the virtual 3-D models shown in virtual pane 1428 created using the creation module or overlay virtual cues on a physical model that is placed on the virtual workbench and registered to the AR system. As described herein, using the edit tab, for example, the user may wish to customize the plate using the virtual 3-D model as reference or modify a graft during a reconstruction surgery. The edit tab provides the user editing tools such as cut, bend, zoom, rotate, draw, color, highlight, modify, annotate, share, select, etc as shown in virtual pane 1426. These features may be accessible via appropriate icons such as scissors for cut, pencil/pen for drawing, magnifying glass for zoom, etc. Upon selecting the appropriate tab, the action is performed on the virtual anatomical model in the virtual pane 1428. Location in the surgical workflow 1418 is also displayed at the bottom of the GUI 1400.

Other actions may also be performed by selecting the edit tab from a plurality of tabs. In an example embodiment, the user may use the select tool from the edit tab to select the location on the virtual medical device 3-D model of a medical device such as the plate where he wishes to cut during the customization phase as described herein. After selecting, user will select the cut tool and the virtual workbench bench will then cut the selected portion of the virtual 3-D medical device and display the result. If the user is satisfied with the result, they may then proceed to the next step at the virtual workbench or on the patient. Any user connected to the AR system may access the edit tab by standing facing the virtual workbench spot/location.

In certain embodiments, the user may select the print tab from a plurality of tabs 1420, as shown in FIG. 14C. Once selected, it will display the second virtual pane with additional sub-tabs 1426. It may also display the status of the connected additive manufacturing device(s) such as 3-D printer(s) and such as available, ready, offline, etc. Using the print tab, the user may be able to select one or more of the sub-tabs 1426 to print items such as anatomical models of the pre-op positions, intra-op (most recently updated version), planned post-op position, template for medical instruments, guides, miscellaneous items as surgical tags, etc. Additionally, while viewing the surgical workflow, the user may decide that they would like to print a physical replica of a virtual 3-D model or a virtual medical device 3-D model in a particular view (top view, side view, etc.). They may be able to do so by dragging and dropping the 3-D model onto the print tab. This is a quick, shortcut to print action without changing any printer settings. Once the printing module accepts the command, it will display the approx. time to print and start printing. This information is always accessible via the printing tools on the second virtual pane. The user may then go back to viewing the surgical workflow. Optionally, as printing may take time, the print tab may also be accessible remotely as long as the user has access to the AR system. The system will notify the user when the printing is finished.

In certain embodiments, the user may select the control tab from a plurality of tabs 1420, as shown in FIG. 14D. Once selected, it will display all connected external systems that may be controlled via the AR system, from the virtual workbench. For example, during the surgery, the surgeon may wish to use a robotic arm. Once the connected robotic arm is selected, a virtual model of the robotic arm is displayed in the virtual pane 1428 which can then be controlled via hand-based gestures or (haptic) hand movements. Any movement made on the virtual model will be transmitted to the physical robot located in the OR. This not only offloads the pressure of controlling a robotic arm at the surgical table, but also leads to less crowding around the surgical table as the person who is controlling the robot doesn't have to be physically present near the robot to control it. This is especially beneficial in long surgeries where a large number of surgeons and supporting staff are involved.

In certain embodiments, the methods disclosed herein include generating a virtual scene comprising one or more virtual elements comprising one or more anatomical elements corresponding to one or more anatomical parts and/or one or more instrument elements corresponding to one or more medical devices. The method further includes identifying one or more references in an actual physical scene comprising the one or more anatomy part and/or instruments and/or medical devices. The method further includes registering the virtual scene to the one or more references to generate an augmented scene. The method further includes guiding elements corresponding to conversion of standard instruments, medical devices (such as implants), grafts to custom (or personalized) versions. The method further includes displaying the augmented scene on a display device.

Certain embodiments provide a non-transitory computer-readable medium having computer-executable instructions stored thereon, which, when executed by a processor of a computing device, cause the computing device to perform the described method.

Certain embodiments provide a computing device comprising a memory and a processor configured to perform the described method.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the invention, which is defined in the accompanying claims. Further, it is to be understood that the methods, systems, and devices disclosed herein may be used in parts or in combination with other standard systems that may be capable of running the methods or parts of the methods or parts of the system on their platform.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims. Further, one or more blocks/steps may be removed or added.

The method and the steps thereon may be performed manually, i.e. by a user, automatically by a computing device, or semi-automatically.

It is to be understood that various embodiments and aspects disclosed herein provide for the use of a system, preferably an AR system. It is to be understood that several modules of the systems disclosed herein may be run independently or configured to run as parts of different compatible systems.

Certain embodiments comprise any of the devices, such as physical template, medical device, implants, described herein.

Certain embodiments comprise a combination of any of standard implants described herein and any surgical navigation system described herein.

Certain embodiments comprise surgical navigation systems configured to execute any of the methods described herein.

It is to be understood that various embodiments and aspects disclosed herein provide for the use of devices that may be shaped using one or more methods disclosed herein or using a system disclosed herein or a combination of both.

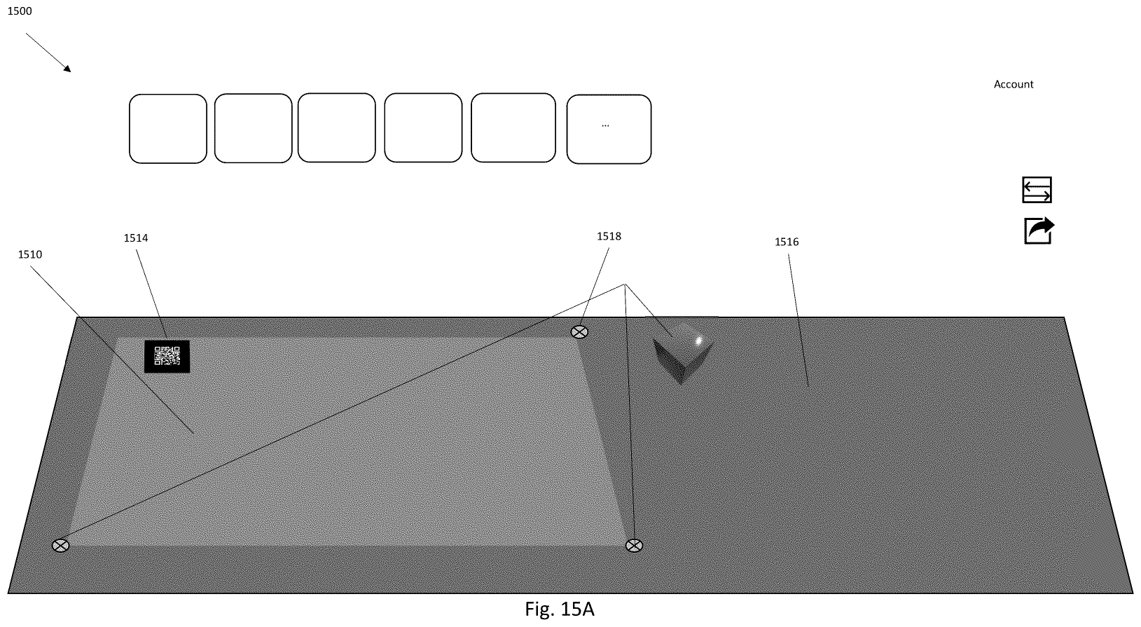

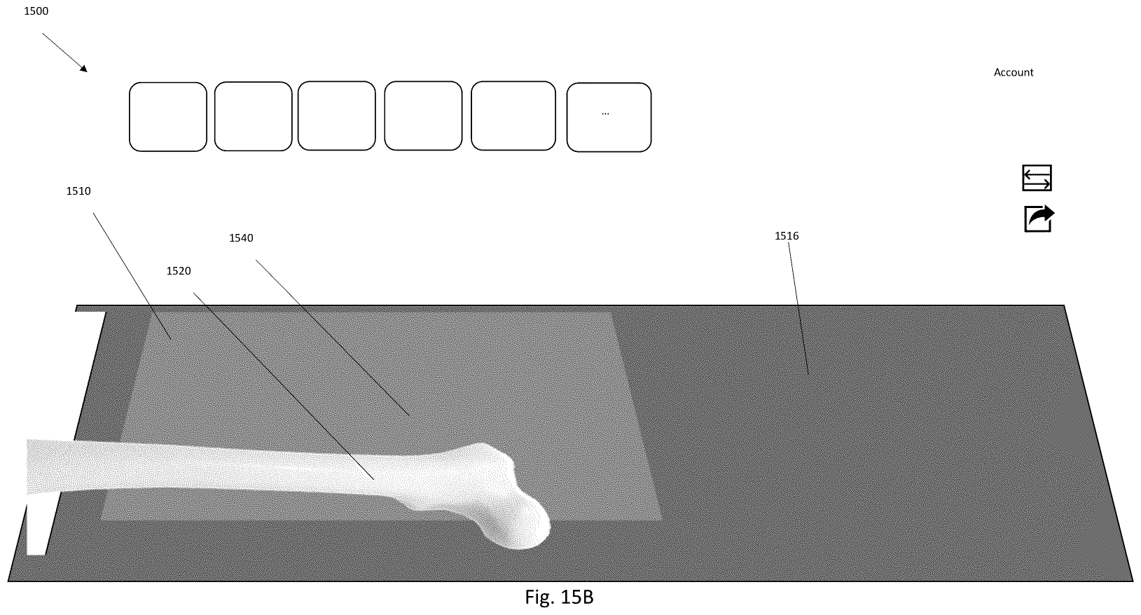

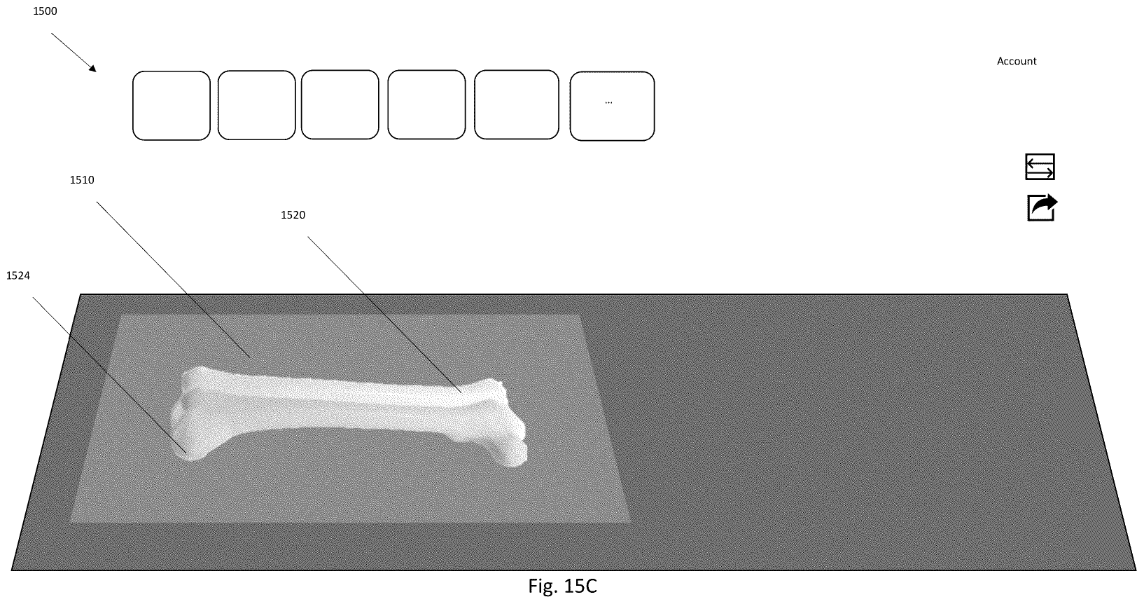

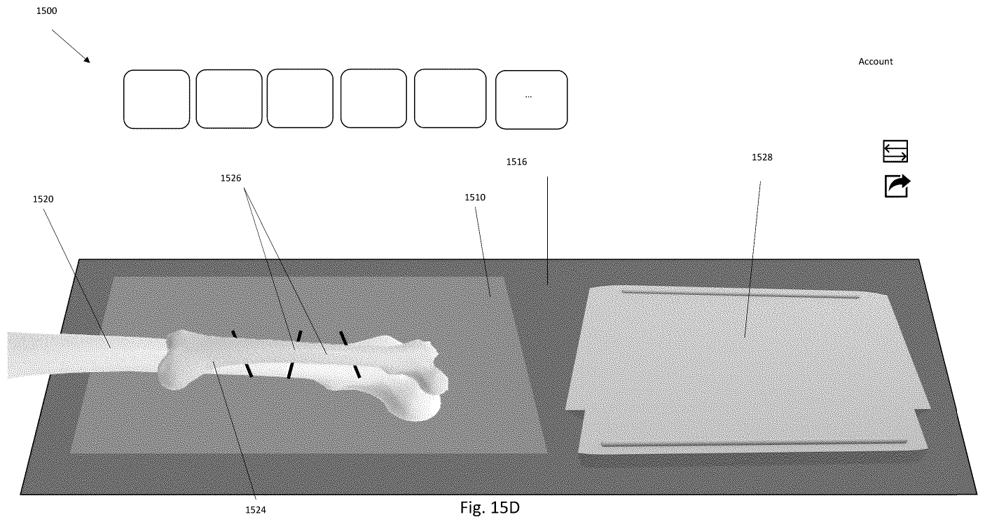

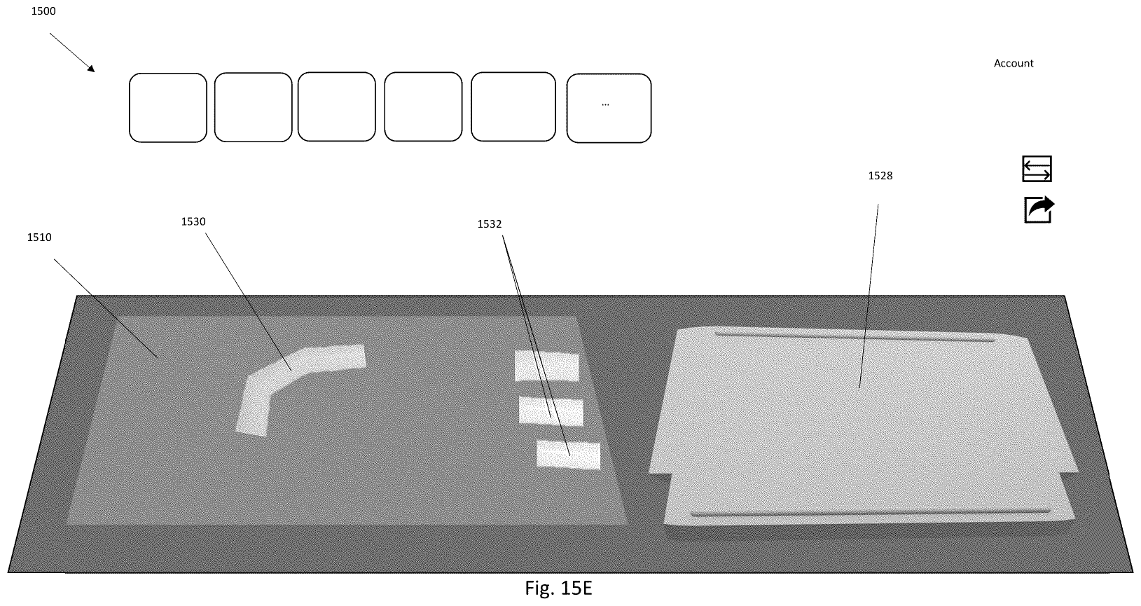

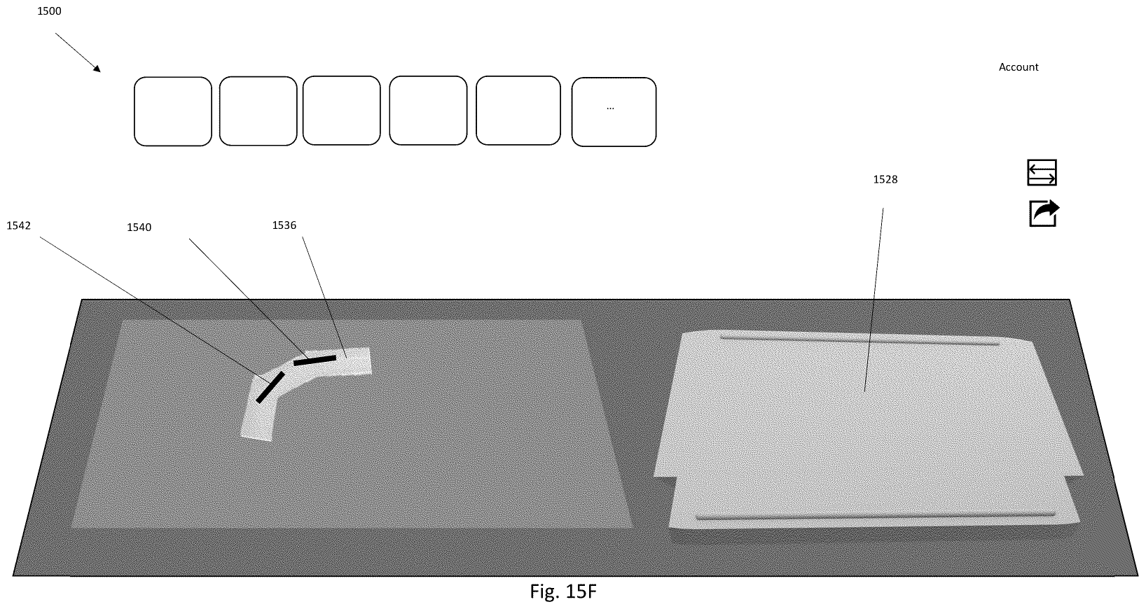

FIGS. 15A-15F, illustrates aspects of a process wherein reconstructing a bone graft is done at the virtual workbench for a reconstruction surgery of a mandible, according to certain embodiments.

Step 1: As shown in FIG. 15A, the virtual workbench 1510 is accessed by a user using one of the available visual cues 1514 such as by scanning QR that is placed on a nearby, conveniently, preselected physical location such as surgical table 1516. The surgical table 1516 is placed in the vicinity of the user such that it is close by but doesn't interfere with the user's movements. In any case, as the virtual workbench 1510 is fixed in spatial location to the surgical table 1516, it can be easily moved to another location by simply placing the surgical table in a different location. This way, it is always available and easy to find and is not free-floating in the virtual environment. Additional physical tracker elements 1518 such as known 2-D markers, special recognition tools etc. may be used for tracking other items that are placed on the surgical tray 1516. All the tabs 1320/1420, from the system described in FIGS. 13 and 14 may be available to the user at all times.

Step 2: As shown in FIG. 15B, the harvested fibula graft 1520 is placed at the virtual workbench 1510. The fibula graft 1520 is tracked using a physical tracker such as a tag, marker, etc. (not shown). Other types of grafts as described earlier in the application may also be used such as an allograft, an autograft, a synthetic bone graft, etc. Once tagged with a tracker, the fibula graft can now be tracked via the AR system, as shown in FIG. 15C.

Step 3: the AR system may now register the virtual anatomical model of the graft 1524 to the harvested physical graft 1520 and can now track it in real-time. The registration may be performed by using any known registration method as described herein such as shape recognition or manually by the user using the virtual workbench 1510. At the virtual workbench 1510, a user may now be able to access stored pre-op plan information, specifically related to graft reconstruction phase. The plan (or aspects of it) necessary for guiding grafting are virtually overlaid on the fibular graft.

Step 4: In FIG. 15D, upon overlay, the user can now see how the physical graft 1520 needs to be shaped. Virtual cues 1526 for cutting, drilling, shaping are visualized on the virtual fibula graft model 1524. For example, virtual drill cylinders 1526 are shown at locations where drilling is required or cut slots 1526 are shown where the graft needs to be cut. Alternatively, virtual cues may be visualized on a real size virtual anatomical model of a fibula graft that has been stored in the storage module (not shown). On the other side of the surgical table, physical surgical tray 1528 with instruments (not shown) required for shaping may be present. These instruments along with the virtual cues 1526 can guide a user during the grafting process. For example, the drill bit for drilling, cutting instrument, etc. may be present on the physical surgical tray 1528.

The user may wish to view these instructions and carry on with the surgery. Alternatively, the user may choose for step-by step guidance

Step 5: the user shapes the fibula graft 1520 in accordance with the post-op outcome using the virtual cues 1526 provided at the virtual workbench 1510.

Step 6: As shown in FIG. 15E, a virtual model of a reconstructed graft 1530 is shown. Each piece of the harvested fibula 1532 may be tracked individually. Alternatively, on the virtual workbench 1510, a dedicated spot for keeping the cut pieces may be placed.

Step 7: As shown in FIG. 15F, at the virtual workbench 1510, a user is guided to place medical device for reconstruction of the graft. In this view, a combined view 1536 of a virtual anatomical model 1524 and the physical shaped graft 1520 is shown. Also shown are virtual medical device model 1542 and a physical medical device 1540 that will be used in reconstructing the graft. This way, a user can verify the position of a medical device 1540 virtually before the actual reconstruction. The physical medical device 1540 (a plate) may also be registered and tracked, similar to the graft.

Step 8: Post visualization, visual cues are now provided to proceed with the fixation such as instruments to be used, drilling of holes, etc. (similar to previous steps).

Step 9: the graft and the medical device are now prepared for placement in the patient.

Step 10: the user may no longer use the virtual workbench and export the remainder of the guidance to the OHMD device for assistance during fixation on the patient. Alternatively, the user may not like wish to use the AR system at all. In that case, the user has to simply leave the location of the virtual workbench 1510. In any case, if the user wishes to refer to any information, it is available at the virtual workbench 1510 which is still fixed on the surgical table 1516 and easily accessible to the user by merely standing in front of it.

### AR System

FIG. 21 illustrates a flow chart showing a process 2100 for operating an augmented reality system, according to certain embodiments. Process 2100 may be performed by a computing device, such as device 300.

Process 2100 begins at block 2102, by generating a virtual scene and registering it to a physical scene to generate an augmented scene. At block 2104, pre-operative measurements are measured in the augmented scene. For example, the measurements may include one or more of the distances between individual bones, the dental occlusion, tissue properties or soft-tissue attachments, etc. In certain embodiments, the measurements are used to update a pre-operative surgical plan, such as by providing the measurements to a modeling or machine learning system that adapts planning parameters of the pre-operative surgical plan. The adapted planning parameters may be used to update the virtual scene and, potentially without re-performing the registration, the augmented scene is updated.

At block 2106, the augmented scene may be visualized to the user, such as prior to performing the surgery, such as a cut. At block 2108, the user may provide inputs based on the visualization to adapt the planning parameters, and the process may return to block 2106 to visualize the updated augmented scene accordingly.

At block 2110, a surgeon may execute the plan by performing drilling and/or cutting as part of a surgery. At block 2112, after executing the plan, additional evaluation measurements can be performed. These may allow to evaluate the execution, e.g., by evaluating the expected outcome based on the actual position of the implant components. Based on this information, further plan adaptations can be suggested at block 2108 and 2106 and the surgeon may wish to redo certain parts of the procedure at block 2110, e.g., re-cutting certain bones. When the final plan has been executed, the augmented reality system may be used to perform post-operative measurements at block 2114.

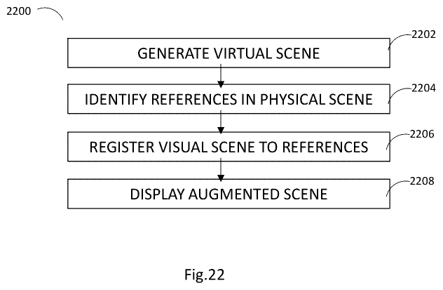

FIG. 22 illustrates a flow chart showing a process 2200 for operating an augmented reality system such as to provide augmented reality assisted surgery, according to certain embodiments. Process 2200 may be performed by a computing device, such as device 300.

Process 2200 begins at block 2202, by generating a virtual scene comprising one or more virtual elements comprising one or more anatomical elements corresponding to one or more anatomy parts. Further, at block 2204, one or more references are identified in an actual physical scene comprising the one or more anatomy parts.

Continuing, at block 2206, the virtual scene is registered to the one or more references to generate an augmented scene. At block 2208, the augmented scene is displayed on a display device.

In certain embodiments, process 2200 further includes acquiring one or more images of the actual physical scene.

In certain embodiments, the one or more references comprise a patient-specific guide placed on the one or more anatomy parts.

In certain embodiments, process 2200 further includes acquiring at least one image of the one or more anatomy parts; and segmenting the at least one image to generate one or more virtual 3-D models of the one or more anatomy parts.

In certain embodiments, process 2200 further includes acquiring one or more virtual 3-D models of the one or more anatomy parts; determining one or more implant components based on the one or more virtual 3-D models; determining a size and a position for the one or more implant components based on the one or more virtual 3-D models; and wherein the one or more anatomical elements comprise a depiction of the one or more implant components having the determined size in the determined position.

In certain embodiments, the one or more anatomical elements comprise portions of the one or more anatomy parts obscured in the one or more images.

In certain embodiments, the one or more anatomical elements comprise highlights corresponding to the one or more anatomy parts.

In certain embodiments, the one or more references comprise physical markers or objects in the actual physical scene.

In certain embodiments, the one or more references comprise landmarks on the one or more anatomy parts.

In certain embodiments, input of the one or more references is received via a marking device.

In certain embodiments, input of the one or more references is received via a surgical plan.

In certain embodiments, process 2200 further includes acquiring one or more virtual 3-D models of the one or more anatomy parts, wherein the one or more references are automatically determined by performing shape recognition on the one or more virtual 3-D models and the one or more images of the physical scene.

In certain embodiments, process 2200 further includes performing one or more measurements of the one or more anatomy parts based on the one or more images, wherein the one or more anatomical elements comprise the one or more measurements.

In certain embodiments, the one or more anatomical elements comprise guidance for one or more steps of a surgical procedure, and the one or more anatomical elements are displayed in an order corresponding to the steps of the surgical procedure.

In certain embodiments, the one or more anatomical elements comprise planned or simulated instrument trajectories for performing surgery on the one or more anatomy parts.

In certain embodiments, process 2200 further includes determining an alignment between an instrument in the one or more images and a planned trajectory for the instrument, wherein the one or more anatomical elements indicate the alignment.

In certain embodiments, process 2200 further includes determining, based on the one or more images or another input, which step of a plurality of steps of a surgery the one or more images corresponds to; and generating at least one of the one or more anatomical elements that belong to the determined step based on the determined step.

1. A method for augmented reality for surgery, comprising:
using a physical registration object to determine a reference coordinate system for an augmented reality scene, the physical registration object comprising at least one of a surgical implant, surgical instrument, or a surgical guide for the surgery;
receiving a surgical plan comprising an indication of desired relative position of one or more virtual objects with respect to at least the physical registration object in the augmented reality scene, the one or more virtual objects comprising at least part of an anatomy of a patient;
determining, using a sensor, a position of the physical registration object in the augmented reality scene; and
displaying the one or more virtual objects within the augmented reality scene at the desired relative position with respect to the physical registration object based on the surgical plan.

2. The method of claim 1, wherein the physical registration object is a cutting guide.

3. The method of claim 1, wherein the at least part of an anatomy of the patient comprises bone.

4. The method of claim 1, wherein the at least part of an anatomy of the patient comprises soft tissue.

5. The method of claim 1, wherein the at least part of an anatomy of the patient comprises anatomical landmarks.

6. The method of claim 1, wherein the one or more virtual objects further comprise surgical screws.

7. The method of claim 1, wherein the one or more virtual objects comprise a drilling trajectory.

8. The method of claim 1, wherein the one or more virtual objects comprise a cutting plane.

9. The method of claim 1, wherein the one or more virtual objects further comprise safety zones or margins where a procedure may be executed.

10. The method of claim 1, wherein the physical registration objection comprises a physical plate for craniomaxillofacial surgery, wherein the one or more virtual objects comprise a series of plate shapes, and wherein displaying the one or more virtual objects comprises displaying a first plate shape of the series of plate shapes based on determining, using the sensor, that the physical plate is in a first configuration and displaying a second plate shape of the series of plate shapes based on determining that the physical plate is in a second configuration.

11. A method for augmented reality for craniomaxillofacial surgery, comprising:
using a physical registration object to determine a reference coordinate system for an augmented reality scene, the physical registration object comprising a physical plate for craniomaxillofacial surgery;
receiving a surgical plan comprising an indication of one or more locations of bending of the physical plate, and for each of the one or more locations a degree and direction of bending of the physical plate;
determining, using a sensor, a position of the physical registration object in the augmented reality scene; and
displaying within the augmented reality scene, aligned with the physical plate, one or more virtual objects indicating the corresponding degree and direction of bending at each of the one or more locations.

12. The method of claim 11, wherein the one or more virtual objects comprise a series of plate shapes, and wherein displaying the one or more virtual objects comprises displaying a first plate shape of the series of plate shapes based on determining, using the sensor, that the physical plate is in a first configuration and displaying a second plate shape of the series of plate shapes based on determining that the physical plate is in a second configuration.

13. A method for visualizing a virtual workbench in a surgical setting, comprising:
detecting a physical marker in a surgical room; and
displaying a virtual workbench in an augmented reality scene on a display device at a position relative to the physical marker, wherein the position of the virtual workbench remains fixed with respect to the marker as the display device moves in the surgical room;
wherein the virtual workbench comprises one or more interfaces configured to selectively display one or more of:
a series of guidance steps for performing a surgery;
tools for registering or overlaying virtual objects to physical objects; or
tools for modifying, measuring or annotating virtual or physical objects.

14. The method of claim 13, wherein the one or more interfaces are further configured to selectively display data obtained from, or controls belonging to, external systems.

15. The method of claim 13, wherein the one or more interfaces are further configured to selectively display a visualization of anatomical data, anatomical images, or a surgical plan.

16. The method of claim 13, wherein the series of guidance steps for performing the surgery comprises a series of plate shapes for guiding bending of a physical plate for craniomaxillofacial surgery.

17. The method of claim 13, wherein the series of guidance steps for performing the surgery comprises a series of steps for graft harvesting.

18. The method of claim 13, wherein the series of guidance steps for performing the surgery comprises a series of steps for bone fragment rearrangement.

19. The method of claim 13, wherein the virtual workbench further comprises an interface for causing a virtual surgical device displayed in the augmented reality scene to be manufactured using additive manufacturing.

20. The method of claim 13, wherein the one or more interfaces are further configured to selectively display a true-to-scale virtual object in relation to one or more physical objects.

21. The method of claim 13, further comprising communicating patient data modified using the virtual workbench to another computing device.

22. The method of claim 13, wherein the other computing device comprises a second virtual workbench.
